(19) United States
(12) Patent Application Publication
(10) Pub. No.: US 2026/0258138 A1
(43) Pub. Date: Sep. 3, 2026

(54) METHODS FOR TREATING CHRONIC INFLAMMATORY DEMYELINATING POLYNEUROPATHY

(71) Applicant: argenx BV, Ghent (BE)

(72) Inventors: Erik HOFMAN, Ghent (BE); Wim PARYS, Ekeren (BE); Manolo BEELKE, Herscheid (DE); Stéphanie DINCQ, Ghent (BE); Murray LOWE, West Kilbride (GB); Katarina SEGHERS, Ghent (BE); Els DE PAEPE, Ghent (BE); Benjamin VAN HOORICK, Ghent (BE); Geoffrey ISTAS, Ghent (BE); Ming JIANG, Ghent (BE); Jeffrey GUPTILL, Ghent (BE); Jon BEAUCHAMP, Ghent (BE); Luc TRUYEN, Ghent (BE)

(21) Appl. No.: 19/448,259

(22) Filed: Jan. 14, 2026

Related U.S. Application Data
(63) Continuation of application No. PCT/IB2024/000374, filed on Jul. 16, 2024.
(60) Provisional application No. 63/617,702, filed on Jan. 4, 2024.
(60) Provisional application No. 63/662,098, filed on Jun. 20, 2024.

(30) Foreign Application Priority Data
Jul. 16, 2023 (GB) 2310890.5

(51) Int. Cl. C07K 16/28; A61P 25/02

(52) U.S. Cl. CPC C07K 16/283; A61P 25/02

(57) ABSTRACT

The present invention relates to methods of treating Chronic Inflammatory Demyelinating Polyneuropathy (CIDP) using an antagonist of human neonatal Fc receptor (FcRn), which in certain embodiments is efgartigimod.

### CROSS REFERENCE TO RELATED APPLICATIONS

This application is a continuation of International Patent Application No. PCT/IB2024/000374, filed Jul. 16, 2024, which claims the benefit of and priority to UK Patent Application No. 2310890.5, filed Jul. 16, 2023, U.S. Provisional Patent Application No. 63/617,702, filed Jan. 4, 2024, and U.S. Provisional Patent Application No. 63/662,098, filed Jun. 20, 2024; the entire contents of which are hereby incorporated by reference in their entireties.

### SUBMISSION OF SEQUENCE LISTING XML

The content of the electronically submitted Sequence Listing XML (Name: 404373-T2307WO_SL.xml; Size: 40,694 bytes; Created on Jul. 11, 2024) is incorporated by reference herein in its entirety.

### FIELD

The present invention relates to methods of treating Chronic Inflammatory Demyelinating Polyneuropathy (CIDP) using an antagonist of human neonatal Fc receptor (FcRn), which in certain embodiments is efgartigimod.

### BACKGROUND

CIDP is the most common chronic immune-mediated inflammatory polyneuropathy with an estimated incidence of around 0.8-8.9 cases per 100,000 individuals (Lehmann et al., 2019). CIDP rates increase with advancing age and the average age at onset is 48 years. CIDP predominantly affects men, the male:female ratio being approximately 2:1 (Vallat et al., 2010).

CIDP is a symmetric sensorimotor disorder with cytoalbuminologic dissociation and interstitial and perivascular endoneurial infiltration by lymphocytes and macrophages. Clinically, CIDP most commonly has an insidious onset with either a chronic progressive or a relapsing course.

CIDP is a highly heterogeneous condition. Multiple variants of CIDP have been described that have immune or inflammatory aspects and electrophysiologic and/or pathologic evidence of demyelination in common. No consensus exists on the best approach for the nomenclature of these disorders. CIDP variants include disorders with predominantly sensory symptoms, distal symmetric disorder or distal acquired demyelinating symmetric neuropathy (DADS), multifocal acquired demyelinating sensory and motor neuropathy (MADSAM), and CIDP with associated central nervous system (CNS) demyelination or with other systemic disorders (Lehmann et al., 2019). As a consequence of the heterogeneity that exists in this disease indication, diagnosis is very challenging and misdiagnosis is common (Van den Bergh et al., 2021).

The underlying pathogenesis of CIDP is poorly understood. The abiding theory is that cellular immune mechanisms together with humoral mechanisms act synergistically to cause damage to peripheral nerves. CIDP nerve biopsies have revealed the infiltration of inflammatory cells including CD8⁺ T cells, CD4⁺ T cells and macrophages. The presence of antibodies to myelin proteins and nodal antigens has also been detected; however, there is only limited direct evidence for pathogenic antibodies in the broad CIDP population. The heterogeneity in CIDP is thought to be a reflection of different immunological mechanisms at play in patients with this condition (Mathey et al., 2015).

For patients with CIDP, the most common treatments are currently immunosuppressive or immunomodulatory interventions. Intravenous immunoglobulin (IVIg) is typically the first-line treatment in most patients. IVIg is a relatively non-specific form of treatment having the potential to mediate its therapeutic effects via multiple modes of action. In the treatment of CIDP, the therapeutic effects of IVIg are thought to be mediated via a combination of: (i) anti-idiotype antibody activity; (ii) saturation of FcRn; (iii) anti-complement activity; (iv) upregulation of inhibitory FcγRIIb receptors that inhibit macrophage activation; and (v) downregulation of co-stimulatory and adhesion molecules (Dalakas et al., 2022). The relative importance of these mechanisms is largely unknown. IVIg is typically administered to subjects at high dose with maintenance doses every 2-6 weeks (Bunschoten et al., 2019). This dosing regimen is suggestive of a more indirect mechanism of action for IVIg in CIDP treatment.

Other commonly used therapeutics include subcutaneous immunoglobulin (SCIg), corticosteroids, plasmapheresis, plasma exchange, prednisone, azathioprine, methotrexate, mycophenolate, cyclosporine, and cyclophosphamide.

Despite the treatments available, a significant number of patients experience continued progression of their disease in the form of relapses or progressive disability. In the case for IVIg/SCIg, infusions require hospital attendance and long-term treatment, which is often costly. Corticosteroids are often more cost-effective but are associated with adverse side-effects (Kuwabara et al., 2006). Accordingly, an unmet medical need exists for a new efficacious treatment for CIDP.

### SUMMARY

The clinical trial results presented herein establish, for the first time, the efficacy of an FcRn antagonist in the treatment of CIDP. As explained above, there has, up to this point, been no consensus on the pathology underlying CIDP. Indeed, CIDP has long been recognised as a highly heterogeneous condition. Treatment with relatively non-specific immunosuppressive therapies, particularly IVIg, is regarded by many experts in the field as beneficial in CIDP due to the multiple modes of action of such therapies (Dalakas et al., 2022). The data reported herein show that an FcRn antagonist, efgartigimod, is effective in improving the symptoms of patients with CIDP and also in preventing relapse following an improvement in symptoms. These effects were seen across a broad population of CIDP patients having different baseline characteristics. These results establish, for the first time, that blockade of FcRn activity via the use of FcRn antagonists is an effective therapeutic approach to the treatment of CIDP.

It follows, that in a first aspect, the present invention provides a method of treating CIDP in a subject in need thereof, the method comprising administering to the subject an effective amount of a FcRn antagonist. Also encompassed within this first aspect is an FcRn antagonist for use in the treatment of CIDP according to any of the methods described herein. Also encompassed within this first aspect is use of an FcRn antagonist in the manufacture of a medicament for the treatment of CIDP wherein the treatment is carried out according to any of the methods described herein.

In some embodiments, the FcRn antagonist comprises two, three, or four FcRn binding regions.

In some embodiments, the FcRn antagonist comprises or consists of a variant Fc region or FcRn binding fragment thereof. In some embodiments, the variant Fc region or FcRn binding fragment thereof binds to FcRn with a higher affinity at pH 6.0 as compared to a corresponding wild-type Fc region. Alternatively or in addition, the variant Fc region or FcRn binding fragment thereof binds to FcRn with a higher affinity at pH 7.4 as compared to a corresponding wild-type Fc region.

In some embodiments, the variant Fc region comprises or consists of a first Fc domain and a second Fc domain which form a homodimer or heterodimer. In some embodiments, the first Fc domain and/or the second Fc domain comprise amino acids Y, T, E, K, and F at EU positions 252, 254, 256, 433, and 434, respectively. In some embodiments, the first Fc domain and/or second Fc domain comprise amino acids Y, T, E, K, F, and Y at EU positions 252, 254, 256, 433, 434, and 436, respectively.

In some embodiments, the first Fc domain and/or the second Fc domain comprise an amino acid sequence independently selected from the group consisting of SEQ ID NO: 1, SEQ ID NO: 2, SEQ ID NO: 3, and SEQ ID NO: 4. In some embodiments, the first Fc domain and the second Fc domain comprise an amino acid sequence independently selected from the group consisting of SEQ ID NO: 1, SEQ ID NO: 2, SEQ ID NO: 3, and SEQ ID NO: 4.

In some embodiments, the FcRn antagonist is efgartigimod.

In some embodiments, the FcRn antagonist is an anti-FcRn antibody.

In some embodiments, the FcRn antagonist is administered to the subject at a fixed dose of 200 mg to 20,000 mg. In some embodiments, the FcRn antagonist is administered to the subject at a dose of 2 mg/kg to 200 mg/kg. In some embodiments, the FcRn antagonist is administered to the subject at a fixed dose of about 800 to about 1200 mg. In some embodiments, the FcRn antagonist is administered to the subject at a fixed dose of about 1000 mg.

In some embodiments, the FcRn antagonist is administered subcutaneously.

In some embodiments, the FcRn antagonist is administered to the subject once weekly. In some embodiments, the FcRn antagonist is administered to the subject once every two weeks. In some embodiments, the FcRn antagonist is administered to the subject once every two weeks, once every three weeks, once every four weeks, once monthly, or once every six weeks.

In some embodiments, the FcRn antagonist is administered subcutaneously at a fixed dose of about 1000 mg once weekly.

In some embodiments, the FcRn antagonist is administered in an induction phase followed by a maintenance phase, wherein the FcRn antagonist is administered subcutaneously once weekly at a fixed dose of about 1000 mg during the induction phase, and wherein the FcRn antagonist is administered subcutaneously once every two weeks at a fixed dose of about 1000 mg during the maintenance phase.

In some embodiments, the FcRn antagonist is first administered subcutaneously at a fixed dose of about 1000 mg once weekly and is subsequently administered subcutaneously at a fixed dose of about 1000 mg once every two weeks based on clinical evaluation. In some embodiments, once weekly subcutaneous administrations at a fixed dose of about 1000 mg are resumed upon worsening of symptoms.

In some embodiments, the FcRn antagonist is administered intravenously. In some embodiments, the FcRn antagonist is administered intravenously once weekly or once every two weeks. In some embodiments, the FcRn antagonist is administered at a dose of from about 3 mg/kg to about 60 mg/kg. In some embodiments, the FcRn antagonist is administered at a dose of 5 mg/kg, 10 mg/kg, or 25 mg/kg.

In some embodiments, the FcRn antagonist is first administered intravenously and is subsequently administered subcutaneously.

In some embodiments, the FcRn antagonist is administered for 61 weeks or less, 52 weeks or less, or 48 weeks or less. In some embodiments, the FcRn antagonist is administered for at least 12 weeks.

In some embodiments, the FcRn antagonist is administered subcutaneously once weekly for at least 4 weeks. In some embodiments, the FcRn antagonist is administered subcutaneously once weekly until the subject demonstrates evidence of clinical improvement (ECI). In some embodiments, the FcRn antagonist is administered subcutaneously once weekly until the subject demonstrates ECI during two consecutive measurements. In some embodiments, the FcRn antagonist is administered subcutaneously once weekly until the subject demonstrates ECI twice in two weeks. In some embodiments, ECI is a clinical improvement in one or more of Inflammatory Rasch-built Overall Disability Scale (I-RODS), Mean Grip Strength, or Inflammatory Neuropathy Cause and Treatment (INCAT) score. In some embodiments, the FcRn antagonist is administered subcutaneously once weekly until the subject demonstrates evidence of clinically meaningful deterioration (ECMD). In some embodiments, the ECMD is one or more of an increase in adjusted INCAT (aINCAT) score of ≥1 points, a decrease in I-RODS of ≥4 points (using the centile metric), or a decrease in mean grip strength of ≥8 kPa in one hand using a handheld vigorimeter. In some embodiments, the ECMD is an increase in aINCAT score of ≥1 points, optionally an increase in aINCAT score of ≥1 points during two consecutive measurements, optionally an increase in aINCAT score of ≥1 points twice in two weeks. In some embodiments, the ECMD is an increase in aINCAT score of ≥2 points.

In some embodiments, the subject has been diagnosed with CIDP according to the EFNS/PNS 2010 diagnostic criteria. In some embodiments, the subject has typical CIDP. In some embodiments, the subject has a CIDP variant, optionally a CIDP variant selected from distal CIDP, multifocal CIDP, focal CIDP, motor CIDP and sensory CIDP.

In some embodiments, the subject has progressive CIDP or relapsing CIDP.

In some embodiments, the subject has an aINCAT score, prior to administration of the FcRn antagonist, of 2 or more, optionally 3 or more, optionally 4 or more, optionally 5 or more.

In some embodiments, the subject has an I-RODS centile metric score, prior to administration of the FcRn antagonist, of 10 or more, optionally 20 or more, optionally 30 or more, optionally 40 or more, optionally 50 or more.

In some embodiments, the subject is newly-diagnosed with CIDP. In some embodiments, the subject is treatment-naïve. In some embodiments, the subject has previously received treatment for CIDP, optionally steroid treatment, optionally IVIg or SCIg treatment. In some embodiments, the subject has previously received treatment for CIDP, optionally steroid treatment, optionally IVIg or SCIg treatment, but has not received treatment for CIDP in the 6 months preceding the start of the treatment with the FcRn antagonist. In some embodiments, the subject has active disease despite treatment with corticosteroids or immunoglobulins.

In some embodiments, the subject is a CIDP patient characterized by the presence of autoantibodies, for example anti-NF155 antibodies, anti-CNTN1 antibodies, anti-Caspr1 antibodies, anti-NF140/186 antibodies, anti-GM-1 antibodies and/or anti-LM-1 antibodies.

In some embodiments, the control of disease activity, partial remission, or complete remission is achieved following administration of the FcRn antagonist. In some embodiments, the control of disease activity, partial remission, or complete remission is achieved within 12 weeks or less, optionally within 8 weeks or less, optionally within 6 weeks or less, optionally within 4 weeks or less, optionally within 3 weeks or less, of first receiving the FcRn antagonist. In some embodiments, the control of disease activity, partial remission, or complete remission is sustained in the subject for at least 2 months or at least 6 months.

In some embodiments, once the control of disease activity, partial remission, or complete remission is achieved, continued administration of the FcRn antagonist prevents deterioration of symptoms. The absence of a deterioration of symptoms may be measured using any one of the following: the INCAT score; the Medical Research Council (MRC) sum score; the I-RODS; the Mean Grip Strength test; or the Timed Up and Go (TUG) test.

In some embodiments, the treatment prevents or delays relapse. In some embodiments, the treatment prevents or delays relapse for at least 4 weeks, at least 6 weeks, at least 8 weeks, at least 10 weeks, at least 12 weeks, at least 16 weeks, or at least 20 weeks.

In some embodiments, the treatment prevents or delays relapse for the duration of the treatment with the FcRn antagonist.

In some embodiments, the treatment prevents or delays relapse following cessation of treatment with the FcRn antagonist. In some embodiments, the treatment prevents or delays relapse following cessation of the treatment for at least 4 weeks, at least 6 weeks, at least 8 weeks, at least 10 weeks, at least 12 weeks, at least 16 weeks, or at least 20 weeks. In some embodiments, the treatment reduces the risk of relapse by at least 60%.

In some embodiments, the subject shows an improvement in symptoms following administration of the FcRn antagonist, as measured using the INCAT score, preferably as measured using the aINCAT score. In some embodiments, the subject shows a decrease in aINCAT score of 1 or more points, optionally 2 or more points.

In some embodiments, the subject shows an improvement in symptoms following administration of the FcRn antagonist, as measured using the MRC sum score.

In some embodiments, the subject shows an improvement in symptoms following administration of the FcRn antagonist, as measured using the I-RODS. In some embodiments, the subject shows an increase in I-RODS centile metric score of 4 or more points.

In some embodiments, the subject shows an improvement in symptoms following administration of the FcRn antagonist, as measured using the Mean Grip Strength test. In some embodiments, the subject shows an increase in mean grip strength of ≥8 kPa.

In some embodiments, the subject shows an improvement in symptoms following administration of the FcRn antagonist, as measured using the TUG test.

In some embodiments, an improvement in symptoms is achieved within 12 weeks or less, optionally within 8 weeks or less, optionally within 6 weeks or less, optionally within 4 weeks or less, optionally within 3 weeks or less, of first receiving the FcRn antagonist.

In some embodiments, the subject shows a reduction in a serum level of total IgG, an autoantibody, a cytokine/chemokine, or an immune complex following administration of the FcRn antagonist. In some embodiments, the serum level of total IgG, the autoantibody, the cytokine/chemokine, or the immune complex is measured 4 weeks, 12 weeks, 24 weeks, or 48 weeks following administration of the FcRn antagonist. In some embodiments, the subject shows a reduction in serum levels of one or more autoantibodies selected from the group consisting of: anti-GM1, anti-LM-1, Anti-NF-155, anti-CNTN1, anti-Caspr-1 antibodies, and anti-myelinated nerve antibodies, following administration of the FcRn antagonist.

In some embodiments, the subject does not show a decrease in the level of serum albumin following administration of the FcRn antagonist. In some embodiments, the subject does not show an increase in serum cholesterol following administration of the FcRn antagonist.

In some embodiments, the subject demonstrates ECI of first receiving the FcRn antagonist. In some embodiments, the subject demonstrates ECI within 31-51 days of first receiving the FcRn antagonist. In some embodiments, the subject demonstrates ECI within 43 days of first receiving the FcRn antagonist.

In some embodiments, the method further comprises administering to the subject an effective amount of one or more additional therapeutic agents, optionally a corticosteroid.

In some embodiments, the method is used to assist in the diagnosis of CIDP.

In another aspect, provided herein is a method for treating CIDP in subjects in a patient population comprising administering 1008 mg/11,200 units of efgartigimod PH20, or a biosimilar version thereof, once weekly, wherein the patient population demonstrates ECI in 66.5% of the subjects in the patient population following administration of the efgartigimod PH20, or biosimilar version thereof.

In some embodiments, the patient population achieves ECI within 31-51 days of first receiving the efgartigimod PH20, or biosimilar version thereof. In some embodiments, the patient population achieves ECI within 43 days of first receiving the efgartigimod PH20, or biosimilar version thereof.

In some embodiments, the patient population comprises 322 subjects.

In some embodiments, anti-efgartigimod alfa antibodies are detected in 6% of the patient population following administration of the efgartigimod PH20 for up to 12 weeks. In some embodiments, neutralizing anti-efgartigimod alfa antibodies are detected in 0.3% of the patient population following administration of the efgartigimod PH20 for up to 12 weeks.

In some embodiments, the subjects in the patient population remain relapse-free significantly longer than subjects who did not receive efgartigimod PH20, or a biosimilar version thereof.

In some embodiments, the subjects in the patient population experience a longer time to clinical deterioration compared to subjects who did not receive efgartigimod PH20, or a biosimilar version thereof, wherein the clinical deterioration is an increase of ≥1 point in aINCAT score. In some embodiments, the clinical deterioration is an increase of ≥1 point in aINCAT score during two consecutive measurements. In some embodiments, the clinical deterioration is an increase of ≥2 points in aINCAT score. In some embodiments, the longer time to clinical deterioration is demonstrated by a hazard ratio of 0.394. In some embodiments, the longer time to clinical deterioration is statistically significant.

In some embodiments, the subjects in the patient population treated with efgartigimod PH20, or a biosimilar version thereof, demonstrate a 61% risk reduction for deterioration in patients with CIDP.

In another aspect, provided herein is a method for treating CIDP in a subject comprising: administering 1008 mg/11,200 units of efgartigimod PH20, or a biosimilar version thereof, once weekly, wherein the subject demonstrates ECI following administration of the efgartigimod PH20, or biosimilar version thereof. In some embodiments, the subject achieves the ECI within 31-51 days of first receiving the efgartigimod PH20, or biosimilar version thereof. In some embodiments, the subject achieves the ECI within 43 days of first receiving the efgartigimod PH20, or biosimilar version thereof. In some embodiments, the subject remains relapse-free for at least 4 weeks, at least 6 weeks, at least 8 weeks, at least 10 weeks, at least 12 weeks, at least 16 weeks, or at least 20 weeks following administration of the efgartigimod PH20, or biosimilar version thereof. In some embodiments, the subject demonstrates a reduced risk of showing ECMD. In some embodiments, the ECMD is an increase in aINCAT score of ≥1 points, optionally an increase in aINCAT score of ≥1 points during two consecutive measurements, optionally an increase in aINCAT score of ≥1 points twice in two weeks. In some embodiments, the ECMD is an increase in aINCAT score of ≥2 points.

In another aspect, provided herein is a method for treating CIDP in subjects in a patient population comprising administering 1008 mg/11,200 units of efgartigimod PH20, or a biosimilar version thereof, once weekly, wherein mean percentage reduction from baseline in total IgG levels ranged between 66.8% and 71.6% in the patient population following administration of the efgartigimod PH20, or biosimilar version thereof. In some embodiments, the mean percentage reduction from baseline in total IgG levels ranged between 66.8% and 71.6% in the patient population following 4 weekly administrations of the efgartigimod PH20, or biosimilar version thereof. In some embodiments, the mean percentage reduction from baseline in total IgG levels was sustained from week 4 throughout the treatment period. In some embodiments, the patient population comprises 322 subjects.

In another aspect, provided herein is a method for treating CIDP in a subject comprising: administering 1008 mg/11,200 units of efgartigimod PH20, or a biosimilar version thereof, once weekly, wherein the subject shows a reduction in a serum level of total IgG of between 66.8% and 71.6% following administration of the efgartigimod PH20, or biosimilar version thereof, compared to a baseline value prior to administration of the efgartigimod PH20, or biosimilar version thereof. In some embodiments, the subject shows a reduction in serum level of total IgG of between 66.8% and 71.6% following 4 weekly administrations of the efgartigimod PH20, or biosimilar version thereof. In some embodiments, the reduction in serum level of total IgG is sustained until once weekly administration of the efgartigimod PH20, or biosimilar version thereof, is discontinued.

In another aspect, provided herein is a method for treating CIDP in subjects in a patient population comprising: administering 1008 mg/11,200 units of efgartigimod PH20, or a biosimilar version thereof, once weekly, wherein the subjects in the patient population experience a longer time to clinical deterioration compared to subjects who did not receive efgartigimod PH20, or a biosimilar version thereof, which is statistically significant, wherein the clinical deterioration is an increase of ≥1 point in aINCAT score. In some embodiments, the longer time to clinical deterioration is demonstrated by a hazard ratio of 0.394. In some embodiments, the patient population comprises 221 subjects.

In another aspect, provided herein is a method of treating CIDP in a subject in need thereof, the method comprising subcutaneously administering to the subject a clinically proven safe and a clinically proven effective amount of efgartigimod PH20 once weekly.

In some embodiments, the clinically proven safe and clinically proven effective amount of efgartigimod PH20 is administered subcutaneously over approximately 30 to 90 seconds. In some embodiments, the clinically proven safe and clinically proven effective amount of efgartigimod PH20 is 1008 mg/11,200 units.

In some embodiments, the subject demonstrates evidence of improvement following administration of the efgartigimod PH20. In some embodiments, the subject demonstrates evidence of improvement during two consecutive measurements. In some embodiments, the evidence of improvement is selected from aINCAT improvement 1 point, I-RODS improvement ≥4 points, or mean grip strength improvement ≥8 kPa.

In some embodiments, the subject experiences a longer time to clinical deterioration following administration of the efgartigimod PH20 as compared to a subject that does not receive efgartigimod PH20. In some embodiments, the longer time to clinical deterioration is statistically significant. In some embodiments, the clinical deterioration is an increase in aINCAT score of ≥1 points, optionally an increase in aINCAT score of ≥1 points during two consecutive measurements, optionally an increase in aINCAT score of 1 points twice in two weeks. In some embodiments, the clinical deterioration is an increase in aINCAT score of 2 points.

In another aspect, provided herein is a method of treating CIDP in subjects in a patient population, the method comprising subcutaneously administering a clinically proven safe and a clinically proven effective amount of efgartigimod PH20 once weekly.

In some embodiments, the clinically proven safe and clinically proven effective amount of efgartigimod PH20 is administered subcutaneously over approximately 30 to 90 seconds. In some embodiments, the clinically proven safe and clinically proven effective amount of efgartigimod PH20 is 1008 mg/11,200 units.

In some embodiments, the patient population demonstrates evidence of improvement during two consecutive measurements in 69% of the subjects in the patient population following administration of the efgartigimod PH20. In some embodiments, the evidence of improvement is selected from aINCAT improvement ≥1 point, I-RODS improvement ≥4 points, or mean grip strength improvement ≥8 kPa.

In some embodiments, the subjects in the patient population experience a longer time to clinical deterioration compared to subjects who did not receive efgartigimod PH20, or a biosimilar version thereof, wherein the clinical deterioration is an increase of ≥1 point in aINCAT score. In some embodiments, the clinical deterioration is an increase of ≥1 point in aINCAT score during two consecutive measurements. In some embodiments, the clinical deterioration is an increase of ≥2 points in aINCAT score. In some embodiments, the longer time to clinical deterioration is demonstrated by a hazard ratio of 0.394. In some embodiments, the longer time to clinical deterioration is statistically significant. In some embodiments, the patient population comprises 221 subjects.

In another aspect, efgartigimod PH20 is provided herein for use in the treatment of CIDP in a subject in need thereof, wherein a clinically proven safe and clinically proven effective amount of the efgartigimod PH20 is subcutaneously administered to the subject once weekly or once every other week. In some embodiments, the clinically proven safe and clinically proven effective amount of the efgartigimod PH20 is administered subcutaneously once weekly.

In some embodiments, the clinically proven safe and clinically proven effective amount of the efgartigimod PH20 is initially administered subcutaneously once weekly and is subsequently administered subcutaneously once every other week based on clinical evaluation. In some embodiments, once weekly subcutaneous administrations are resumed upon worsening of symptoms.

In some embodiments, the clinically proven safe and clinically proven effective amount of the efgartigimod PH20 is administered subcutaneously over approximately 30 to 90 seconds.

In some embodiments, the clinically proven safe and clinically proven effective amount of the efgartigimod PH20 comprises 1000 mg efgartigimod alfa.

In some embodiments, the subject has active disease despite treatment with corticosteroids or immunoglobulins.

In some embodiments, the subject received a prior CIDP therapy before initiation of efgartigimod PH20 treatment, and wherein the efgartigimod PH20 is administered before a clinical effect of the prior CIDP therapy decreases. In some embodiments, the prior CIDP therapy is corticosteroids or immunoglobulins.

In some embodiments, the subject shows a reduction in serum level of total IgG of between 66.8% and 71.6% following administration of the efgartigimod PH20, compared to a baseline value prior to administration of the efgartigimod PH20. In some embodiments, the subject shows a reduction in serum level of total IgG of between 66.8% and 71.6% following 4 weekly administrations of the efgartigimod PH20. In some embodiments, the reduction in serum level of total IgG is sustained until once weekly administration of the efgartigimod PH20 is discontinued.

In some embodiments, the efgartigimod PH20, when administered to a patient population of CIDP subjects, induces an ECI response in 66.5% of the subjects in the patient population.

In some embodiments, the subject demonstrates ECI following administration of the efgartigimod PH20. In some embodiments, the subject demonstrates ECI within 31-51 days of first receiving the efgartigimod PH20. In some embodiments, the subject demonstrates ECI within 43 days of first receiving the efgartigimod PH20.

In some embodiments, the subject remains relapse-free significantly longer following administration of the efgartigimod PH20 as compared to a subject that does not receive efgartigimod PH20.

In some embodiments, the subject demonstrates a reduced risk of showing evidence of clinical deterioration following administration of the efgartigimod PH20. In some embodiments, the evidence of clinical deterioration is an increase in aINCAT score of ≥1 points. In some embodiments, the evidence of clinical deterioration is an increase in aINCAT score of ≥1 points during two consecutive measurements. In some embodiments, the evidence of clinical deterioration is an increase in aINCAT score of ≥1 points twice in two weeks. In some embodiments, the evidence of clinical deterioration is an increase in aINCAT score of ≥2 points.

In another aspect, efgartigimod PH20 is provided herein for use in the treatment of CIDP in subjects in a patient population, the treatment comprising subcutaneously administering a clinically proven safe and a clinically proven effective amount of the efgartigimod PH20 once weekly or once every other week. In some embodiments, the clinically proven safe and clinically proven effective amount of the efgartigimod PH20 is administered subcutaneously once weekly. In some embodiments, the clinically proven safe and clinically proven effective amount of the efgartigimod PH20 is initially administered subcutaneously once weekly and is subsequently administered subcutaneously once every other week based on clinical evaluation. In some embodiments, once weekly subcutaneous administrations are resumed upon worsening of symptoms.

In some embodiments, the clinically proven safe and clinically proven effective amount of the efgartigimod PH20 is administered subcutaneously over approximately 30 to 90 seconds.

In some embodiments, the clinically proven safe and clinically proven effective amount of the efgartigimod PH20 comprises 1000 mg efgartigimod alfa.

In some embodiments, the subjects in the patient population have active disease despite treatment with corticosteroids or immunoglobulins.

In some embodiments, the subjects in the patient population received a prior CIDP therapy before initiation of the efgartigimod PH20 treatment, and wherein the efgartigimod PH20 is administered before a clinical effect of the prior CIDP therapy decreases. In some embodiments, the prior CIDP therapy is corticosteroids or immunoglobulins.

In some embodiments, mean percentage reduction from baseline in total IgG levels ranged between 66.8% and 71.6% in the patient population following administration of the efgartigimod PH20. In some embodiments, the mean percentage reduction from baseline in total IgG levels ranged between 66.8% and 71.6% in the patient population following 4 weekly administrations of the efgartigimod PH20. In some embodiments, the mean percentage reduction from baseline in total IgG levels was sustained from week 4 throughout the treatment period.

In some embodiments, the patient population demonstrates ECI in 66.5% of the subjects in the patient population following administration of the efgartigimod PH20. In some embodiments, the patient population achieves the ECI within 31-51 days of first receiving the efgartigimod PH20. In some embodiments, the patient population achieves the ECI within 43 days of first receiving the efgartigimod PH20.

In some embodiments, the subjects in the patient population remain relapse-free significantly longer than subjects who did not receive efgartigimod PH20.

In some embodiments, the subjects in the patient population treated with efgartigimod PH20 demonstrate a 61% risk reduction for deterioration.

In some embodiments, the subjects in the patient population demonstrate a reduced risk of showing evidence of clinical deterioration following administration of the efgartigimod PH20. In some embodiments, the evidence of clinical deterioration is an increase in aINCAT score of ≥1 points. In some embodiments, the evidence of clinical deterioration is an increase in aINCAT score of ≥1 points during two consecutive measurements. In some embodiments, the evidence of clinical deterioration is an increase in aINCAT score of ≥1 points twice in two weeks. In some embodiments, the evidence of clinical deterioration is an increase in aINCAT score of ≥2 points.

In some embodiments, the patient population comprises 322 subjects.

In some embodiments, the subject in any of the methods or uses described herein is an adult human.

In another aspect, provided herein is a drug product approved for treatment of CIDP comprising efgartigimod and hyaluronidase, wherein the drug product is formulated for subcutaneous administration. In some embodiments, the drug product is formulated for subcutaneous administration over approximately 30 to 90 seconds. In some embodiments, the drug product is administered in a total volume of 5.6 mL.

In some embodiments, the drug product is provided in a single-dose vial at a concentration of 180 mg efgartigimod/2000 units hyaluronidase per mL. In some embodiments, the drug product is provided in a single-dose vial containing 180 mg/mL efgartigimod, 2,000 units/mL recombinant human hyaluronidase (rHuPH20), 1.4 mg/mL histidine, 2.2 mg/mL L-histidine hydrochloride monohydrate, 1.5 mg/mL methionine, 0.4 mg/mL polysorbate 20, 5.8 mg/mL sodium chloride, 20.5 mg/mL sucrose, and water for injection, USP, in a total volume of 5.6 mL, at a pH of 6.0.

In some embodiments, the drug product is reference listed on the basis of efgartigimod administered at a dose of 1000 mg. In some embodiments, the drug product is reference listed on the basis of efgartigimod administered at a dose of 1008 mg. In some embodiments, the drug product is reference listed on the basis of hyaluronidase administered at a dose of 11,200 units.

In some embodiments, the drug product is reference listed on the basis of once weekly or once every other week administration. In some embodiments, the drug product is reference listed on the basis of once weekly administration. In some embodiments, the drug product is reference listed on the basis of once weekly administration, followed by adjustment to once every other week administration based on clinical evaluation. In some embodiments, the drug product is reference listed on the basis of resumption of once weekly administration in case of worsening of symptoms.

In some embodiments, the drug product is approved for administration to an adult human patient. In some embodiments, the drug product has an approved indication for treatment of CIDP in adult human patients with active disease despite treatment with corticosteroids or immunoglobulins.

In some embodiments, the drug product is contraindicated in patients with serious hypersensitivity to efgartigimod alfa products, to hyaluronidase, or to any excipients of the drug product.

In some embodiments, the drug product induces improvement at two consecutive visits in 69% of a population of CIDP patients who received 1008 mg/11,200 units of efgartigimod PH20 once weekly up to 12 weeks. In some embodiments, the improvement is aINCAT improvement ≥1 point, I-RODS improvement ≥4 points, or mean grip strength improvement ≥8 kPa.

In some embodiments, the drug product induces an ECI response in 66.5% of a population of CIDP patients who received 1008 mg/11,200 units of efgartigimod PH20 once weekly. In some embodiments, median time to initial confirmed ECI in the population of CIDP patients is 43 days from first administration of the drug product. In some embodiments, up to 40% of the CIDP patients in the population of CIDP patients had an ECI response four weeks from first administration of the drug product. In some embodiments, 25% of the CIDP patients in the population of CIDP patients showed clinically relevant improvement in at least one of the following: aINCAT score, I-RODS, or mean grip strength, within 9 days of first administration of the drug product. In some embodiments, the population of CIDP patients comprises 322 CIDP patients. In some embodiments, the CIDP patients in the population of CIDP patients who received the drug product remained relapse-free significantly longer than the CIDP patients in the population of CIDP patients who received placebo. In some embodiments, the CIDP patients in the population of CIDP patients who received the drug product demonstrated a 61% risk reduction for deterioration compared to the CIDP patients in the population of CIDP patients who received placebo.

In some embodiments, the CIDP patients in the population of CIDP patients experience a longer time to clinical deterioration compared to CIDP patients in the population of CIDP patients who received placebo. In some embodiments, the clinical deterioration is an increase of ≥1 point in aINCAT score. In some embodiments, the clinical deterioration is an increase of ≥1 point in aINCAT score during two consecutive measurements. In some embodiments, the clinical deterioration is an increase of ≥2 points in aINCAT score. In some embodiments, the longer time to clinical deterioration is demonstrated by a hazard ratio of 0.394. In some embodiments, the longer time to clinical deterioration is statistically significant.

In some embodiments, the drug product induces a hypersensitivity reaction selected from the group consisting of anaphylaxis and hypotension leading to syncope. In some embodiments, the anaphylaxis or hypotension leading to syncope occurs during or within 1 hour of administration of the drug product.

In some embodiments, the drug product induces an infusion-related reaction. In some embodiments, the infusion-related reaction comprises one or more of the following: hypertension, chills, shivering, thoracic pain, abdominal pain, and back pain. In some embodiments, the infusion-related reaction occurs during or within 1 hour of administration of the drug product. In some embodiments, one or more subsequent doses of the drug product are administered with close clinical observation, slower infusion rates, and pre-medications when a mild-to-moderate infusion-related reaction occurs during administration of the drug product.

In some embodiments, the drug product induces anti-efgartigimod antibodies in 2% of subjects in a population of 117 CIDP patients following treatment with the drug product. In some embodiments, the drug product induces anti-efgartigimod antibodies in 6% of subjects in a population of 317 CIDP patients following treatment with the drug product. In some embodiments, the drug product induces neutralizing anti-efgartigimod antibodies in 0.3% of subjects in a population of 317 CIDP patients following treatment with the drug product.

Also provided is a biosimilar of the drug product described above and herein.

Also provided is a biological product that is bioequivalent to the drug product described above and herein.

In another aspect, provided herein is a kit comprising: any drug product described above and herein, any biosimilar described above and herein, or any biological product described above and herein, and a label including an indication for the treatment of CIDP in adult patients.

In some embodiments, the label includes a contraindication in patients with serious hypersensitivity to efgartigimod alfa products, to hyaluronidase, or to any excipients in formulations thereof. In some embodiments, the label includes a warning for hypersensitivity reactions selected from anaphylaxis and hypotension leading to syncope. In some embodiments, the label states infusion discontinuation when anaphylaxis or hypotension leading to syncope occur during or within 1 hour of administration of the product. In some embodiments, the label includes a warning for infusion-related reactions selected from hypertension, chills, shivering, thoracic pain, abdominal pain, and back pain. In some embodiments, the label states to initiate appropriate therapy when a severe infusion-related reaction occurs during administration of the product. In some embodiments, the label states that patients may be rechallenged with close clinical observation, slower infusion rates, and pre-medications when a mild-to-moderate infusion-related reaction occurs during administration of the product.

In some embodiments, wherein the label includes data demonstrating improvement at two consecutive visits in 69% of a population of CIDP patients who received 1008 mg/11,200 units of efgartigimod PH20 once weekly up to 12 weeks. In some embodiments, the improvement is aINCAT improvement ≥1 point, I-RODS improvement ≥4 points, or mean grip strength improvement ≥8 kPa.

In some embodiments, the label includes data demonstrating an ECI response in 66.5% of a population of CIDP patients who received 1008 mg/11,200 units of efgartigimod PH20 once weekly. In some embodiments, the label includes data demonstrating the median time to initial confirmed ECI in a population of CIDP patients is 43 days from first administration of efgartigimod PH20. In some embodiments, the label states that at week 4, the earliest time point for which ECI criteria could have been met, up to 40% of patients had EC. In some embodiments, the label states that 25% of patients showed clinically relevant improvement after 9 days in at least one of the 3 parameters (aINCAT, I-RODS, or Grip Strength). In some embodiments, the label includes data demonstrating CIDP patients in a population of CIDP patients who received efgartigimod PH20 remained relapse-free significantly longer than CIDP patients in a population of CIDP patients who received placebo. In some embodiments, the label includes data demonstrating CIDP patients in a population of CIDP patients who received efgartigimod PH20 demonstrated a 61% risk reduction for deterioration compared to CIDP patients in a population of CIDP patients who received placebo. In some embodiments, the label includes data demonstrating CIDP patients who received efgartigimod PH20 experienced a longer time to clinical deterioration (i.e., increase of ≥1 point in aINCAT score) compared to CIDP patients in a population of CIDP patients who received placebo. In some embodiments, the clinical deterioration is an increase of ≥2 points in aINCAT score. In some embodiments, the longer time to clinical deterioration is demonstrated by a hazard ratio of 0.394. In some embodiments, the longer time to clinical deterioration is statistically significant.

In some embodiments, the label includes data indicating a 2% incidence of anti-efgartigimod alfa antibodies in a population of 117 CIDP patients following treatment with the drug product. In some embodiments, the label includes data indicating a 6% incidence of anti-efgartigimod alfa antibodies in a population of 317 CIDP patients following treatment with the drug product. In some embodiments, the label includes data indicating a 0.3% incidence of neutralizing anti-efgartigimod alfa antibodies in a population of 317 CIDP patients following treatment with the drug product.

In another aspect, provided herein is a method of treating CIDP in a subject in need thereof, the method comprising administering any drug product described above and herein, any biosimilar described above and herein, or any biological product described above and herein to the subject.

### BRIEF DESCRIPTION OF DRAWINGS

FIG. 1 is a schematic of the 2-part Phase 2 study to investigate the efficacy, safety, tolerability, immunogenicity, PK, and PD of efgartigimod PH20 SC (efgartigimod coformulated with recombinant human hyaluronidase PH20 (rHuPH20) for subcutaneous (SC) injection) administered SC in patients aged 18 years and older with CIDP. d=day; OLE=open-label extension; placebo=placebo with rHuPH20; Q1W=once weekly; SC=subcutaneous(ly); w=week.

FIG. 2 is the questionnaire used in the I-RODS.

FIGS. 3A-3B shows results from Stage B of the study, specifically measurement of the primary endpoint: Time to First aINCAT Score Deterioration, in placebo (lower line) and efgartigimod (upper line) treated groups. These results are presented for the primary population of the trial i.e., the mITT analysis set. FIG. 3A: Kaplan-Meier plot of the results. FIG. 3B: Calculations of the median Time to First aINCAT Score Deterioration and the percentage of clinical deterioration seen at 24 weeks and 48 weeks during the stage B study.

FIGS. 4A-4B shows results from Stage B of the study, specifically measurement of the primary endpoint: Time to First aINCAT Score Deterioration, in placebo (lower line) and efgartigimod (upper line) treated groups. These results are presented for the Per Protocol Analysis Set i.e., all patients in the study who fully adhered to the trial protocol. FIG. 4A: Kaplan-Meier plot of the results. FIG. 4B: Calculations of the median Time to First aINCAT Score Deterioration and the percentage of clinical deterioration seen at 24 weeks and 48 weeks during the stage B study.

FIGS. 5A-5F shows results from Stage B of the study, specifically measurement of the primary endpoint: Time to First aINCAT Score Deterioration, in placebo (lower line) and efgartigimod (upper line) treated groups. Patient groups have been stratified according to prior treatment: (i) with corticosteroids (FIGS. 5A and 5B); (ii) with IVIg or SCIg (FIGS. 5C and 5D); or treatment naïve patients (FIGS. 5E and 5F). FIGS. 5A, 5C and 5E: Kaplan-Meier analysis of the results. FIGS. 5B, 5D and 5F: Calculations of the median Time to First aINCAT Score Deterioration.

FIGS. 6A-6B shows results from Stage B of the study, specifically measurement of a secondary endpoint: Time to CIDP progression, in placebo (lower line) and efgartigimod (upper line) treated groups. These results are presented for the primary population of the trial i.e., the mITT analysis set. Time to CIDP disease progression is defined as the time from first dose of double-blind IMP (placebo or efgartigimod) to the first I-RODS score decrease of ≥4 points compared to Stage B baseline using the centile metric. FIG. 6A: Cox Proportion Hazard Model. FIG. 6B: Hazard Ratio.

### DETAILED DESCRIPTION

The present invention relates to methods of treating CIDP. Chronic Inflammatory Demyelinating Polyneuropathy is also referred to in the literature as Chronic Inflammatory Demyelinating Polyradiculoneuropathy (also abbreviated to CIDP). The methods comprise administering to a subject (e.g., a human subject) an effective amount of an FcRn antagonist. The invention also provides human FcRn antagonists for use in treating CIDP. Further provided are uses of FcRn antagonists in the manufacture of medicaments for the treatment of CIDP. All embodiments of the invention described herein are equally applicable to all aspects of the invention.

### A. Definitions

Unless defined otherwise, all technical and scientific terms used herein have the same meaning as is commonly understood by one skilled in the art in the technical field of the invention.

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “FcRn antagonist” refers to any agent that binds specifically to FcRn and inhibits the binding of immunoglobulin to FcRn (e.g., human FcRn). In an embodiment, the FcRn antagonist is an Fc region (e.g., a variant Fc region disclosed herein) that specifically binds to FcRn through the Fc region and inhibits the binding of immunoglobulin to FcRn. In an embodiment, the FcRn antagonist is not a full-length IgG antibody. In an embodiment, the FcRn antagonist comprises an antigen binding site that binds a target antigen and a variant Fc region. In an embodiment, the FcRn antagonist is an Fc fragment comprising or consisting of an Fc region and lacking an antigen binding site. In an embodiment the term “FcRn antagonist” refers to an antibody or antigen-binding fragment thereof that specifically binds to FcRn via its antigen binding domain or via its Fc region and inhibits the binding of the Fc region of immunoglobulin (e.g., IgG autoantibodies) to FcRn.

As used herein, the terms “antibody” and “antibodies” include full-length antibodies, antigen-binding fragments of full-length antibodies, and molecules comprising antibody CDRs, VH regions, or VL regions. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single-domain antibodies (sdAb), monovalent antibodies, single chain antibodies or single-chain Fvs (scFv), camelid antibodies, affibody molecules, humanized antibodies, VHH fragments, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. Antibodies can be of any type (e.g., IgG, IgE, IgM, IgD, IgA, or IgY), any class (e.g., IgG1, IgG2, IgG3, IgG4, IgA1, or IgA2), or any subclass (e.g., IgG2a or IgG2b) of immunoglobulin molecule.

As used herein, the term “Fc domain” refers to the portion of a single immunoglobulin heavy chain beginning in the hinge region and ending at the C-terminus of the antibody. Accordingly, a complete Fc domain comprises at least a portion of a hinge (e.g., upper, middle, and/or lower hinge region) domain, a CH2 domain, and a CH3 domain. In some embodiments, the term “Fc domain” refers to the portion of a single immunoglobulin heavy chain comprising both the CH2 and CH3 domains of the antibody. In some embodiments, the Fc domain comprises at least a portion of a hinge (e.g., upper, middle, and/or lower hinge region) region, a CH2 domain, and a CH3 domain. In some embodiments, the Fc domain does not include the hinge region.

As used herein, the term “hinge region” refers to the portion of a heavy chain molecule that joins the CH1 domain to the CH2 domain. In some embodiments, the hinge region is at most 70 amino acid residues in length. In some embodiments, this hinge region comprises approximately 11-17 amino acid residues and is flexible, thus allowing the two N-terminal antigen binding regions to move independently. In some embodiments, the hinge region is 12 amino acid residues in length. In some embodiments, the hinge region is 15 amino acid residues in length. In some embodiments, the hinge region is 62 amino acid residues in length. Hinge regions can be subdivided into three distinct domains: upper, middle, and lower hinge domains. The FcRn antagonists of the instant disclosure can include all or any portion of a hinge region. In some embodiments, the hinge region is from an IgG1 antibody. In some embodiments, the hinge region comprises the amino acid sequence of EPKSCDKTHTCPPCP (SEQ ID NO: 31).

As used herein, the term “Fc region” refers to the portion of an immunoglobulin formed by the Fc domains of its two heavy chains. The Fc region can be a wild-type Fc region (native Fc region) or a variant Fc region. A native Fc region is homodimeric. The Fc region can be derived from any native immunoglobulin. In some embodiments, the Fc region is formed from an IgA, IgD, IgE, or IgG heavy chain constant region. In some embodiments, the Fc region is formed from an IgG heavy chain constant region. In some embodiments, the IgG heavy chain constant region is an IgG1, IgG2, IgG3, or IgG4 heavy chain constant region. In some embodiments, the Fc region is formed from an IgG1 heavy chain constant region. In some embodiments, the IgG1 heavy chain constant region comprises a G1m1(a), G1m2(x), G1m3(f), or G1m17(z) allotype, see, e.g., Jefferis and Lefranc, 2009 and de Taeye et al., 2020.

As used herein, the term “variant Fe region” refers to an Fe region with one or more alteration(s) relative to a native Fe region. Alterations can include amino acid substitutions, additions and/or deletions, linkage of additional moieties, and/or alteration of the native glycans. The term encompasses heterodimeric Fc regions where each of the constituent Fe domains is different. The term also encompasses single chain Fc regions where the constituent Fe domains are linked together by a linker moiety.

As used herein the term “FcRn binding fragment” refers to a portion of an Fe region that is sufficient to confer FcRn binding.

As used herein, the term “EU position” refers to the amino acid position in the EU numbering convention for the Fe region described in Edelman, G M et al., Proc. Natl. Acad. USA, 1969; 63, 78-85, and Kabat et al., in “Sequences of Proteins of Immunological Interest,” U.S. Dept. Health and Human Services, 5^{th} edition, 1991.

As used herein, the term “baseline” refers to a measurement in a patient, e.g., in a patient's blood, prior to the first administration (e.g., intravenous or subcutaneous administration) of a treatment (e.g., an FcRn antagonist).

As used herein, the term “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “dose” or “dosing” refers to an amount of an agent administered to a subject in a single administration.

As used herein, the terms “fixed dose” or “flat dose” both refer to a dose that does not vary based upon a characteristic (e.g., body mass, e.g., within a set range; sex; age, e.g., within a set range; etc.) of the subject.

As used herein, the term “control of disease activity” or “CDA” refers to the point at which there is no further clinical deterioration and/or there is ECI.

As used herein, the term “remission” refers to complete remission or partial remission.

As used herein, the term “complete remission” or “CR” refers to the absence of symptoms as measured by standard diagnostic criteria for CIDP.

As used herein, the term “partial remission” or “PR” refers to the situation wherein a patient experiences ECI, but does not experience CR.

As used herein, the term “evidence of clinical improvement” or “ECI” refers to the improvement in symptoms as measured by any of the standard CIDP assessment methods including but not limited to (i) the INCAT Disability Scale; (ii) the MRC Scale (Vanhoutte et al., 2012); (iii) the I-RODS; (iv) the Mean Grip Strength test; and (v) the TUG test. In preferred embodiments, ECI refers to an improvement in symptoms as measured using the INCAT Disability Scale, specifically a decrease in INCAT score or a decrease in aINCAT score. In some embodiments, ECI refers to clinical improvement on the parameters that the patient worsened in the period between screening and stage A (I-RODS, Grip Strength), or clinical improvement in INCAT.

As used herein, the term “relapse” refers to a patient with CIDP who exhibits evidence of clinical deterioration after a period of remission (partial or complete remission). In other words, “relapse” refers to evidence of clinical deterioration in a patient with CIDP after a period of remission (partial or complete remission).

As used herein, the term “deterioration” or “clinical deterioration” refers to any deterioration of CIDP symptoms. The absence of a deterioration of CIDP symptoms may be measured using any one of the following: the INCAT score; the MRC sum score; the I-RODS; the Mean Grip Strength test; or the TUG test. As used herein, the terms “evidence of clinical deterioration,” “evidence of clinically meaningful deterioration”, and “ECMD” can be used interchangeably. In some embodiments, ECMD is defined as an increase in aINCAT score by ≥1 point, and/or a decrease in I-RODS by ≥4 points (using the centile metric), and/or a decrease in mean grip strength by ≥8 kPa in one hand using a handheld vigorimeter. In some embodiments, ECMD is defined as an increase in aINCAT score by 1 point on two occasions within 7 days of each other. In some embodiments, ECMD is defined as an increase in aINCAT score by ≥2 points on one occasion. In some embodiments, ECMD is defined as a 10% decrease in I-RODS.

As used herein, the term “subject” or “patient” or “participant” includes any human or non-human animal. In an embodiment, the subject or patient or participant is a human or non-human mammal. In an embodiment, the subject or patient or participant is a human.

As used herein, the term “about” or “approximately” when referring to a measurable value, such as a dosage, encompasses variations of ±5% of a given value or range, as are appropriate to perform the methods disclosed herein.

As used herein, the term “biosimilar” refers to a biological product that is highly similar to and has no clinically meaningful differences from a reference product. As used herein, the term “reference product” refers to a biological product that has been approved for clinical use. In some embodiments, the reference product is approved in at least one of the U.S., Europe, China, or Japan. A biosimilar may have minor differences in clinically inactive components. A biosimilar may include minor modifications in amino acid sequence when compared to the reference product, such as N- or C-terminal truncations that are not expected to change the biosimilar performance.

As used herein, “no clinically meaningful differences” is determined in terms of safety, purity, and potency. For example, a biosimilar is compared to and evaluated against a reference product to verify that the biosimilar has no clinically meaningful differences in terms of safety, purity, and potency from the reference product. In some embodiments, a determination of no clinically meaningful differences between a biosimilar and a reference product is based upon data derived from: (a) analytical studies that demonstrate that the biological product is highly similar to the reference product notwithstanding minor differences in clinically inactive components; (b) animal studies (including, for example, assessment of toxicity); and/or (c) a clinical study or studies (including, for example, assessment of immunogenicity and pharmacokinetics or pharmacodynamics) sufficient to demonstrate safety, purity, and potency in one or more appropriate conditions of use for which the reference product is licensed and for which licensure is sought for the biosimilar. A biosimilar may be an interchangeable product that may be substituted for the reference product at a pharmacy without intervention of a prescribing healthcare professional. To meet a standard of “interchangeability,” the biosimilar is expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. In some embodiments, the biosimilar utilizes the same mechanisms of action as the reference product for the proposed conditions of use, to the extent the mechanisms are known for the reference product. In some embodiments, the condition or conditions of use prescribed, recommended, or suggested in the labeling proposed for the biosimilar have been previously approved for the reference product. In some embodiments, the route of administration, the dosage form, and/or the strength of a biosimilar are the same as those of the reference product and the biosimilar is manufactured, processed, packed, or held in a facility that meets standards designed to assure that the biosimilar continues to be safe, pure, and potent.

As used herein, the terms “label,” “product label,” or “approved product label” refer to information provided to a patient and/or healthcare provider which provides relevant information regarding the approved product. Such information includes, without limitation, one or more of: the description of the approved product, clinical pharmacology, indications (uses for the approved product), contraindication (who should not take the approved product), warnings, precautions, adverse events (side effects), drug abuse and dependence, dosage and administration, use in pregnancy, use in nursing mothers, use in children and older patients, how the approved product is supplied, safety information for the patient, or any combination thereof. In some embodiments, the label identifies efgartigimod and provides instructions for its use in a patient.

As used herein, the term “clinically proven effective” refers to a regulatory determination that is made on the basis of clinical efficacy and other data. Efficacy can be measured based on change in the course of the disease in response to an agent of the present disclosure. For example, an FcRn antagonist of the present disclosure (e.g., efgartigimod) is administered to a subject in an amount and for a time sufficient to induce an improvement, preferably a sustained improvement, in at least one indicator that reflects the severity of the disorder that is being treated. Various indicators that reflect the extent of the subject's illness, disease, or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, blood samples, or other test results, and who can also employ questionnaires that are administered to the subject, such as quality-of-life questionnaires developed for a given disease. For example, an FcRn antagonist of the present disclosure can be administered to achieve an improvement in a subject's condition related to CIDP. Improvement can be indicated by an improvement in an index of disease activity, by amelioration of clinical symptoms or by any other measure of disease activity. Improvement in a subject's condition related to CIDP can be measured using any of the standard CIDP assessment methods described herein, such as those used to define EC. In some embodiments, clinical efficacy is defined as demonstrating ECI in a subject. In some embodiments, clinical efficacy is demonstrated by length of time a subject remains relapse-free. In some embodiments, clinical efficacy is demonstrated by risk reduction for clinical deterioration (such as ECMD).

As used herein, the term “clinically proven safe,” as it relates to a dose, dosing regimen, treatment, or method with an FcRn antagonist of the present disclosure (e.g., efgartigimod), refers to a favorable risk:benefit ratio with an acceptable frequency and/or acceptable severity of treatment-emergent adverse events (referred to as AEs or TEAEs) compared to the standard of care or to another comparator. As used herein, “adverse event,” “treatment-emergent adverse event,” and “adverse reaction” mean any harm, unfavorable, unintended, or undesired sign or outcome associated with or caused by administration of a pharmaceutical composition or therapeutic. It is an untoward medical occurrence in a subject administered a medicinal product. However, abnormal values or observations are not reported as adverse events unless considered clinically significant by the investigator. When the harm or undesired outcome of adverse events reaches such a level of severity, a regulatory agency can deem the pharmaceutical composition or therapeutic unacceptable for the proposed use. In particular, “safe” as it relates to a dose, dosing regimen, or treatment with an FcRn antagonist of the present disclosure refers to with an acceptable frequency and/or acceptable severity of adverse events associated with administration of the FcRn antagonist if attribution is considered to be possible, probable, or very likely due to the use of the FcRn antagonist.

As used herein, unless otherwise noted, the term “clinically proven” (used independently or to modify the terms “safe” and/or “effective”) means that it has been proven by a clinical trial wherein the clinical trial has met the approval standards of U.S. Food and Drug Administration (FDA), European Medicines Agency of the European Union (EMA), Pharmaceuticals and Medical Devices Agency of Japan (PMDA), and National Medical Products Administration of China (NMPA). For example, the clinical study may be an adequately sized, randomized, double-blinded study used to clinically prove the effects of the drug.

### B. FcRn Antagonists

FcRn antagonists that are useful in the methods and uses provided herein can include any molecule that binds to and inhibits FcRn, including but not limited to, any anti-FcRn antibody, any anti-FcRn binding region, or any Fc domain or Fc region. In some embodiments, the FcRn antagonists disclosed herein comprise two, three, or four FcRn binding regions, such as an Fc region. In some embodiments, the FcRn antagonists disclosed herein comprise one or more Fc regions in combination with one or more Fab regions.

Any Fc region can be altered to produce a variant Fc region for use in the methods disclosed herein. In general, an Fc region, or FcRn binding fragment thereof, is from a human immunoglobulin. It is understood, however, that the Fc region may be derived from an immunoglobulin of any other mammalian species, including for example, a camelid species, a rodent (e.g., a mouse, rat, rabbit, guinea pig) or non-human primate (e.g., chimpanzee, macaque) species. Moreover, the Fc region or portion thereof may be derived from any immunoglobulin class, including IgM, IgG, IgD, IgA, and IgE, and any immunoglobulin isotype, including IgG1, IgG2, IgG3, and IgG4.

In an embodiment, the Fc region is an IgG Fc region (e.g., a human IgG region). In an embodiment, the Fc region is an IgG1 Fc region (e.g., a human IgG1 region). In an embodiment, the Fc region is a chimeric Fe region comprising portions of several different Fe regions. Suitable examples of chimeric Fc regions are set forth in US 2011/0243966A1, which is incorporated herein by reference in its entirety. A variety of Fc region gene sequences (e.g., human constant region gene sequences) are available in the form of publicly accessible deposits.

An Fc region can be further truncated or internally deleted to produce a minimal FcRn binding fragment thereof. The ability of an Fe-region fragment to bind to FcRn can be determined using any art recognized binding assay (e.g., ELISA).

To enhance the manufacturability of the FcRn antagonists disclosed herein, it is preferable that the constituent Fe regions do not comprise any non-disulfide bonded cysteine residues. Accordingly, in an embodiment, the Fe regions do not comprise a free cysteine residue.

Any Fe variant, or FcRn binding fragment thereof, that binds specifically to FcRn with increased affinity and reduced pH dependence relative to the native (i.e., wild-type) Fe region can be used in the methods disclosed herein. In an embodiment, the variant Fe region comprises amino acid alterations, substitutions, insertions, and/or deletions that confer the desired characteristics.

In some embodiments, the FcRn antagonist comprises or consists of a variant Fe region, or FcRn binding fragment thereof, which binds to FcRn with a higher affinity at pH 5.5 as compared to a corresponding wild-type Fe region. In some embodiments, the FcRn antagonist comprises or consists of a variant Fe region, or FcRn binding fragment thereof, which bind to FcRn with a higher affinity at pH 6.0 and/or at pH 7.4 as compared to a corresponding wild-type Fe region. In some embodiments, the FcRn antagonist comprises or consists of a variant Fe region, or FcRn binding fragment thereof, which bind to FcRn with a higher affinity at both acidic and neutral pH.

In some embodiments, the variant Fe region is derived from the Fe region of any native immunoglobulin. In some embodiments, the native immunoglobulin is a human immunoglobulin. In some embodiments, the immunoglobulin is IgA, IgD, IgE, or IgG. In some embodiments, the immunoglobulin is IgG. In some embodiments, the immunoglobulin is human IgA, human IgD, human IgE, or human IgG. In some embodiments, the immunoglobulin is human IgG. In some embodiments, the IgG is IgG1, IgG2, IgG3, or IgG4. In some embodiments, the human IgG is human IgG1, human IgG2, human IgG3, or human IgG4. In some embodiments, the variant Fe region varies from the human IgG1 Fe region. In some embodiments, the human IgG1 Fe region comprises a G1m1(a), G1m2(x), G1m3(f), or G1m17(z) allotype.

In an embodiment, the variant Fe region, or FcRn binding fragment thereof consists of two Fe domains. In an embodiment, the FcRn antagonist is an Fe region comprising amino acids Y, T, E, K, F, and Y at EU positions 252, 254, 256, 433, 434, and 436, respectively.

In an embodiment, the variant Fe region comprises or consists of a first Fc domain and a second Fc domain which form a homodimer or heterodimer. In an embodiment, the first Fc domain and/or the second Fc domain comprise amino acids Y, T, E, K, and F at EU positions 252, 254, 256, 433, and 434, respectively. In an embodiment, the first Fc domain and/or the second Fc domain comprise amino acids Y, T, E, K, F, and Y at EU positions 252, 254, 256, 433, 434, and 436, respectively.

In some embodiments, the FcRn antagonists disclosed herein comprise or consist of at least one Fc domain, wherein the amino acid sequence of the at least one Fc domain comprises or consists of the amino acid sequence of SEQ ID NO: 22, provided below in Table 1.

TABLE 1

| | SEQ ID |
|---|---|
| Amino Acid Sequence | NO: |
| X₁X₂X₃X₄X₅X₆PPCPAPELLGGPSVFLFPPKPKDTLYITREPEVTCVVVDVSH | 22 |
| EDPEVKFNWYVDGVEVHNAKTKPREEQYNSTYRVVSVLTVLHQDWLNG | |
| KEYKCKVSNKALPAPIEKTISKAKGQPREPQVYTLPPSRDELTKNQVSLTC | |
| LVKGFYPSDIAVEWESNGQPENNYKTTPPVLDSDGSFFLYSKLTVDKSRW | |
| QQGNVFSCSVMHEALKFHYTQKSLSLSPX₇X₈ | |
| wherein: X₁ is D or absent; X₂ is K or absent; X₃ is T or absent; X₄ is H or | |
| absent; X₅ is T or absent; X₆ is C or absent; X₇ is G or absent; X₈ is K or | |
| absent. | |

In some embodiments, the FcRn antagonists disclosed herein comprise or consist of a variant Fe region comprising or consisting of a dimer of a first Fc domain and a second Fc domain, wherein the amino acid sequence of the first and second Fc domain comprises or consists of the amino acid sequence of SEQ ID NO: 22.

In some embodiments, the FcRn antagonists disclosed herein comprise or consist of an amino acid sequence at least 70%, 75%, 80%, 85%, 90%, 95%, 96%, 97%, 98%, 99%, or 100% identical to the amino acid sequence of a variant Fe region comprising or consisting of a dimer of a first Fc domain and a second Fc domain, wherein the amino acid sequence of the first and second Fc domain comprises or consists of an amino acid sequence independently selected from the group consisting of SEQ ID NOs: 1-21 (see Table 2 below). In some embodiments, the dimer is a heterodimer or a homodimer.

TABLE 2

| | SEQ |
|---|---|
| Amino Acid Sequence | ID NO: |
| CPPCPAPELLGGPSVFLFPPKPKDTLYITREPEVTCVVVDVSHEDPEVKFNW | 1 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPG | |
| DKTHTCPPCPAPELLGGPSVFLFPPKPKDTLYITREPEVTCVVVDVSHEDPE | 2 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPGK | |
| DKTHTCPPCPAPELLGGPSVFLFPPKPKDTLYITREPEVTCVVVDVSHEDPE | 3 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPG | |
| CPPCPAPELLGGPSVFLFPPKPKDTLYITREPEVTCVVVDVSHEDPEVKFNW | 4 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPGK | |
| DKTHTCPPCPAPELLGGPSVFLFPPKPKDTLYITREPEVTCVVVDVSHEDPE | 5 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSP | |
| KTHTCPPCPAPELLGGPSVFLFPPKPKDTLYITREPEVTCVVVDVSHEDPEV | 6 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPGK | |
| KTHTCPPCPAPELLGGPSVFLFPPKPKDTLYITREPEVTCVVVDVSHEDPEV | 7 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPG | |
| KTHTCPPCPAPELLGGPSVFLFPPKPKDTLYITREPEVTCVVVDVSHEDPEV | 8 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSP | |
| THTCPPCPAPELLGGPSVFLFPPKPKDTLYITREPEVTCVVVDVSHEDPEVKF | 9 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPGK | |
| THTCPPCPAPELLGGPSVFLFPPKPKDTLYITREPEVTCVVVDVSHEDPEVKF | 10 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPG | |
| THTCPPCPAPELLGGPSVFLFPPKPKDTLYITREPEVTCVVVDVSHEDPEVKF | 11 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSP | |
| TCPPCPAPELLGGPSVFLFPPKPKDTLYITREPEVTCVVVDVSHEDPEVKFN | 12 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| TCPPCPAPELLGGPSVFLFPPKPKDTLYITREPEVTCVVVDVSHEDPEVKFN | 13 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| TCPPCPAPELLGGPSVFLFPPKPKDTLYITREPEVTCVVVDVSHEDPEVKFN | 14 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| PPCPAPELLGGPSVFLFPPKPKDTLYITREPEVTCVVVDVSHEDPEVKFNWY | 15 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPGK | |
| PPCPAPELLGGPSVFLFPPKPKDTLYITREPEVTCVVVDVSHEDPEVKFNWY | 16 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPG | |
| PPCPAPELLGGPSVFLFPPKPKDTLYITREPEVTCVVVDVSHEDPEVKFNWY | 17 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSP | |
| HTCPPCPAPELLGGPSVFLFPPKPKDTLYITREPEVTCVVVDVSHEDPEVKFN | 18 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| HTCPPCPAPELLGGPSVFLFPPKPKDTLYITREPEVTCVVVDVSHEDPEVKFN | 19 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| HTCPPCPAPELLGGPSVFLFPPKPKDTLYITREPEVTCVVVDVSHEDPEVKFN | 20 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| CPPCPAPELLGGPSVFLFPPKPKDTLYITREPEVTCVVVDVSHEDPEVKFNW | 21 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSP | |

In an embodiment, the first Fc domain and/or the second Fc domain comprise an amino acid sequence independently selected from the group consisting of SEQ ID NOs: 1, 2, 3, and 4. In an embodiment, the first Fc domain and the second Fc domain comprise an amino acid sequence independently selected from the group consisting of SEQ ID NOs: 1, 2, 3, and 4.

In some embodiments, the FcRn antagonist comprises a population of FcRn antagonist molecules. In some embodiments, an FcRn antagonist comprising a first Fc domain and a second Fc domain comprising an amino acid sequence independently selected from the group consisting of SEQ ID NOs: 1, 2, 3, and 4 is the predominant FcRn antagonist molecule in the population of FcRn antagonist molecules. In some embodiments, the predominant FcRn antagonist molecule makes up at least 50%, 55%, 60%, 65%, 70%, 75%, 80%, 85%, 90%, 95%, 96%, 97%, 98%, or 99% of the population of FcRn antagonist molecules.

In an embodiment, the amino acid sequence of the Fc domains of the variant Fc region comprises the amino acid sequence of SEQ ID NO: 1. In an embodiment, the amino acid sequence of the Fc domains of the variant Fc region consists of the amino acid sequence of SEQ ID NO: 1.

In an embodiment, the amino acid sequence of the Fc domains of the variant Fc region comprises the amino acid sequence of SEQ ID NO: 2. In an embodiment, the amino acid sequence of the Fc domains of the variant Fc region consists of the amino acid sequence of SEQ ID NO: 2.

In an embodiment, the amino acid sequence of the Fc domains of the variant Fc region comprises the amino acid sequence of SEQ ID NO: 3. In an embodiment, the amino acid sequence of the Fc domains of the variant Fc region consists of the amino acid sequence of SEQ ID NO: 3.

In an embodiment, the amino acid sequence of the Fc domains of the variant Fc region comprises the amino acid sequence of SEQ ID NO: 4. In an embodiment, the amino acid sequence of the Fc domains of the variant Fc region consists of the amino acid sequence of SEQ ID NO: 4.

In an embodiment, the FcRn antagonist consists of a variant Fc region, wherein the variant Fc region comprises two Fc domains, wherein the amino acid sequence of each of the Fc domains is independently selected from SEQ ID NO: 1, SEQ ID NO: 2, SEQ ID NO: 3, and SEQ ID NO: 4.

In certain embodiments, the variant Fc region is a heterodimer, where the constituent Fc domains are different from each other. Methods of producing Fc heterodimers are known in the art (see, e.g., U.S. Pat. No. 8,216,805, which is incorporated by reference herein in its entirety). In an embodiment, the FcRn antagonist consists of a variant Fc region, wherein the variant Fc region consists of two Fc domains which form a heterodimer, wherein the amino acid sequence of each of the Fc domains is independently selected from SEQ ID NO: 1, SEQ ID NO: 2, or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 1, and the amino acid sequence of the second Fc domain consists of or comprises the amino acid sequence of SEQ ID NO: 2 or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 2, and the amino acid sequence of the second Fc domain consists of or comprises the amino acid sequence of SEQ ID NO: 1 or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 3, and the amino acid sequence of the second Fc domain consists of or comprises the amino acid sequence of SEQ ID NO: 1 or SEQ ID NO: 2.

In an embodiment, the FcRn antagonist consists of a variant Fc region, wherein the variant Fe region consists of two Fe domains which form a heterodimer, wherein the amino acid sequence of each of the Fe domains is independently selected from SEQ ID NO: 1, SEQ ID NO: 2, SEQ ID NO: 3, or SEQ ID NO: 4. In an embodiment, the FcRn antagonist consists of or comprises a variant Fc region, wherein the variant Fe region consists of or comprises two Fe domains which form a heterodimer, wherein the amino acid sequence of the first Fc domain consists of or comprises the amino acid sequence of SEQ ID NO: 1, and the amino acid sequence of the second Fc domain consists of or comprises the amino acid sequence of SEQ ID NO: 2, SEQ ID NO: 3, or SEQ ID NO: 4. In an embodiment, the FcRn antagonist consists of or comprises a variant Fc region, wherein the variant Fe region consists of or comprises two Fe domains which form a heterodimer, wherein the amino acid sequence of the first Fc domain consists of or comprises the amino acid sequence of SEQ ID NO: 2, and the amino acid sequence of the second Fc domain consists of or comprises the amino acid sequence of SEQ ID NO: 1, SEQ ID NO: 3, or SEQ ID NO: 4. In an embodiment, the FcRn antagonist consists of or comprises a variant Fe region, wherein the variant Fe region consists of or comprises two Fe domains which form a heterodimer, wherein the amino acid sequence of the first Fc domain consists of or comprises the amino acid sequence of SEQ ID NO: 3, and the amino acid sequence of the second Fc domain consists of or comprises the amino acid sequence of SEQ ID NO: 1, SEQ ID NO: 2, or SEQ ID NO: 4. In an embodiment, the FcRn antagonist consists of or comprises a variant Fe region, wherein the variant Fe region consists of or comprises two Fe domains which form a heterodimer, wherein the amino acid sequence of the first Fc domain consists of or comprises the amino acid sequence of SEQ ID NO: 4, and the amino acid sequence of the second Fc domain consists of or comprises the amino acid sequence of SEQ ID NO: 1, SEQ ID NO: 2, or SEQ ID NO: 3.

In an embodiment, the FcRn antagonist consists of or comprises a variant Fe region, wherein the variant Fe region consists of or comprises two Fe domains which form a homodimer, wherein the amino acid sequence of each of the Fe domains consists of or comprises the amino acid sequence of SEQ ID NO: 1.

In an embodiment, the FcRn antagonist consists of or comprises a variant Fe region, wherein the variant Fe region consists of or comprises two Fe domains which form a homodimer, wherein the amino acid sequence of each of the Fe domains consists of or comprises the amino acid sequence of SEQ ID NO: 2.

In an embodiment, the FcRn antagonist consists of or comprises a variant Fe region, wherein the variant Fe region consists of or comprises two Fe domains which form a homodimer, wherein the amino acid sequence of each of the Fc domains consists of or comprises the amino acid sequence of SEQ ID NO: 3.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 4.

In some embodiments, the FcRn antagonist comprises glycanation on one or both of the Fc domains. In some embodiments, the FcRn antagonist molecules comprise glycanation at EU position 297 on one or both of the Fc domains. In some embodiments, the glycanation comprises an N-glycan. In some embodiments, the N-glycan comprises a GOF N-glycan, G1F N-glycan, G2F N-glycan, or G0 N-glycan.

In some embodiments, the FcRn antagonist comprises or consists of a population of FcRn antagonists, wherein at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, or at least 57% of the population of Fc domains of the FcRn antagonists comprise galactose. In some embodiments, the population comprises or consists of FcRn antagonists, wherein at least 88%, at least 89%, at least 90%, at least 91%, at least 92%, at least 93%, at least 94%, at least 95%, at least 96%, at least 97%, at least 98%, or at least 99% of the population of Fc domains of the FcRn antagonists comprise fucose.

In some embodiments, the FcRn antagonist lacks an amino acid at EU position 441 of one or both Fc domains. In some embodiments, the FcRn antagonist comprises glycine and lysine at EU positions 440 and 441, respectively. In some embodiments, the FcRn antagonist lacks amino acids at EU positions 440 and 441. In some embodiments, the FcRn antagonist comprises amidated proline at EU position 439. In some embodiments, the FcRn antagonist lacks amino acids at EU positions 440 and 441 and comprises amidated proline at EU position 439.

In some embodiments, the FcRn antagonist comprises aspartate, lysine, threonine, histidine, threonine, and cysteine at EU positions 221, 222, 223, 224, 225, and 226, respectively. In some embodiments, the FcRn antagonist lacks an amino acid at EU positions 221, and comprises lysine, threonine, histidine, threonine, and cysteine at EU positions 222, 223, 224, 225, and 226, respectively. In some embodiments, the FcRn antagonist lacks amino acids at EU positions 221 and 222, and comprises threonine, histidine, threonine, and cysteine at EU positions 223, 224, 225, and 226, respectively. In some embodiments, the FcRn antagonist lacks amino acids at EU positions 221-224, and comprises threonine and cysteine at EU positions 225 and 226, respectively. In some embodiments, the FcRn antagonist lacks amino acids at EU positions 221, 222, 223, 224, 225, and 226.

In some embodiments, the FcRn antagonist is a population of FcRn antagonist molecules. In some embodiments, the population of FcRn antagonist molecules comprises or consists of multiple subpopulations of FcRn antagonist molecules. In some embodiments, the population of FcRn antagonist molecules comprises or consists of 2, 3, 4, 5, 6, 7, 8, 9, 10, 11, 12, 13, 14, 15, 16, 17, 18, 19, or 20 subpopulations. In some embodiments, the population of FcRn antagonist molecules comprises or consists of 2, 3, 4, 5, 6, 7, 8, 9, 10, or 11 subpopulations.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 13, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 10, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fe region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one asparagine residue in each FcRn antagonist molecule in the fifth subpopulation are deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 7, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein two amino acid residues, independently selected from a methionine residue or a tryptophan residue, in each FcRn antagonist molecule in the eleventh subpopulation is oxidized.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 13, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 10, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NO: 3, and wherein one asparagine residue in each FcRn antagonist molecule in the fifth subpopulation is deaminated.

In some embodiments, a sixth subpopulation of FcRn antagonist molecules comprises or consists of a variant Fe region comprising or consisting of a dimer of a first Fc domain and a second Fc domain, wherein the amino acid sequence of the first and second Fc domain comprises or consists of the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NO: 3, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 7, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein two amino acid residues, independently selected from a methionine residue or a tryptophan residue in each FcRn antagonist molecule in the eleventh subpopulation is oxidized.

In some embodiments, the population of FcRn antagonist molecules comprises or consists of the first subpopulation combined with one of the second, third, fourth, fifth, sixth, seventh, eighth, ninth, tenth, or eleventh subpopulations. In some embodiments, the population of FcRn antagonist molecules comprises or consists of the first subpopulation combined with two of the second, third, fourth, fifth, sixth, seventh, eighth, ninth, tenth, or eleventh subpopulations. In some embodiments, the population of FcRn antagonist molecules comprises or consists of the first subpopulation combined with three of the second, third, fourth, fifth, sixth, seventh, eighth, ninth, tenth, or eleventh subpopulations. In some embodiments, the population of FcRn antagonist molecules comprises or consists of the first subpopulation combined with four of the second, third, fourth, fifth, sixth, seventh, eighth, ninth, tenth, or eleventh subpopulations. In some embodiments, the population of FcRn antagonist molecules comprises or consists of the first subpopulation combined with five of the second, third, fourth, fifth, sixth, seventh, eighth, ninth, tenth, or eleventh subpopulations. In some embodiments, the population of FcRn antagonist molecules comprises or consists of the first subpopulation combined with six of the second, third, fourth, fifth, sixth, seventh, eighth, ninth, tenth, or eleventh subpopulations. In some embodiments, the population of FcRn antagonist molecules comprises or consists of the first subpopulation combined with seven of the second, third, fourth, fifth, sixth, seventh, eighth, ninth, tenth, or eleventh subpopulations. In some embodiments, the population of FcRn antagonist molecules comprises or consists of the first subpopulation combined with eight of the second, third, fourth, fifth, sixth, seventh, eighth, ninth, tenth, or eleventh subpopulations. In some embodiments, the population of FcRn antagonist molecules comprises or consists of the first subpopulation combined with nine of the second, third, fourth, fifth, sixth, seventh, eighth, ninth, tenth, or eleventh subpopulations. In some embodiments, the population of FcRn antagonist molecules comprises or consists of the first subpopulation combined with all of the second, third, fourth, fifth, sixth, seventh, eighth, ninth, tenth, or eleventh subpopulations.

In some embodiments, the population comprises or consists of the first and second subpopulations. In some embodiments, the population comprises or consists of the first and third subpopulations. In some embodiments, the population comprises or consists of the first and fourth subpopulations. In some embodiments, the population comprises or consists of the first and fifth subpopulations. In some embodiments, the population comprises or consists of the first and sixth subpopulations. In some embodiments, the population comprises or consists of the first and seventh subpopulations. In some embodiments, the population comprises or consists of the first and eighth subpopulations. In some embodiments, the population comprises or consists of the first and ninth subpopulations. In some embodiments, the population comprises or consists of the first and tenth subpopulations. In some embodiments, the population comprises or consists of the first and eleventh subpopulations. In some embodiments, the populations listed above further comprise or consist of 1, 2, 3, 4, 5, 6, 7, 8, or 9 additional subpopulations. In some embodiments, these additional subpopulations are one or more of those described above.

In some embodiments, the population comprises or consists of the first and seventh, ninth, or eleventh subpopulations. In some embodiments, the population comprises or consists of the first, seventh, ninth, and eleventh subpopulations.

In some embodiments, the first subpopulation makes up at least 40%, at least 45%, at least 50%, at least 55%, at least 60%, at least 65%, at least 70%, at least 75%, at least 80%, at least 85%, or at least 90% of the population of FcRn antagonist molecules. In some embodiments, the first subpopulation makes up about 40%, about 45%, about 50%, about 55%, about 60%, about 65%, about 70%, about 75%, about 80%, about 85%, or about 90% of the population of FcRn antagonist molecules. In some embodiments, the first subpopulation makes up 40%, 45%, 50%, 55%, 60%, 65%, 70%, 75%, 80%, 85%, or 90% of the population of FcRn antagonist molecules. In some embodiments, the first subpopulation makes up 40%-90%, 50%-80%, or 55%-70% of the population of FcRn antagonist molecules. In some embodiments, the first subpopulation makes up 56.9%-68.3% or 59.5%-67.9% of the population of FcRn antagonist molecules.

In some embodiments, the second subpopulation makes up less than 3.0%, less than 2.5%, less than 2.0%, less than 1.5%, less than 1%, or less than 0.5% of the population of FcRn antagonist molecules. In some embodiments, the second subpopulation makes up about 3.0%, about 2.5%, about 2.0%, about 1.5%, about 1%, or about 0.5% of the population of FcRn antagonist molecules. In some embodiments, the second subpopulation makes up 3.0%, 2.5%, 2.0%, 1.5%, 1%, or 0.5% of the population of FcRn antagonist molecules. In some embodiments, the second subpopulation makes up 0.5%-3.0%, 1.0%-2.5%, or 1.0%-2.0% of the population of FcRn antagonist molecules. In some embodiments, the second subpopulation makes up 0.8%-2.0% or 0.8%-2.1% of the population of FcRn antagonist molecules.

In some embodiments, the third subpopulation makes up less than 3.0%, less than 2.5%, less than 2.0%, less than 1.5%, less than 1%, or less than 0.5% of the population of FcRn antagonist molecules. In some embodiments, the third subpopulation makes up about 3.0%, about 2.5%, about 2.0%, about 1.5%, about 1%, or about 0.5% of the population of FcRn antagonist molecules. In some embodiments, the third subpopulation makes up 3.0%, 2.5%, 2.0%, 1.5%, 1%, or 0.5% of the population of FcRn antagonist molecules. In some embodiments, the third subpopulation makes up 0.5%-3.0%, 1.0%-2.5%, or 1.0%-2.0% of the population of FcRn antagonist molecules. In some embodiments, the third subpopulation makes up 1.1%-2.1% or 1.0%-1.9% of the population of FcRn antagonist molecules.

In some embodiments, the fourth subpopulation makes up less than 5%, less than 4%, less than 3%, less than 2%, or less than 1% of the population of FcRn antagonist molecules. In some embodiments, the fourth subpopulation makes up about 5%, about 4%, about 3%, about 2%, or about 1% of the population of FcRn antagonist molecules. In some embodiments, the fourth subpopulation makes up 5%, 4%, 3%, 2%, or 1% of the population of FcRn antagonist molecules. In some embodiments, the fourth subpopulation makes up 1%-5%, 2%-4%, or 2%-3% of the population of FcRn antagonist molecules. In some embodiments, the fourth subpopulation makes up 2.1%-3.2% or 2.0%-3.1% of the population of FcRn antagonist molecules.

In some embodiments, the fifth subpopulation makes up less than 12%, less than 11%, less than 10%, less than 9%, less than 8%, less than 7%, less than 6%, or less than 5% of the population of FcRn antagonist molecules. In some embodiments, the fifth subpopulation makes up about 12%, about 11%, about 10%, about 9%, about 8%, about 7%, about 6%, or about 5% of the population of FcRn antagonist molecules. In some embodiments, the fifth subpopulation makes up 12%, 11%, 10%, 9%, 8%, 7%, 6%, or 5% of the population of FcRn antagonist molecules. In some embodiments, the fifth subpopulation makes up 5%-12%, 6%-10%, or 7%-8% of the population of FcRn antagonist molecules. In some embodiments, the fifth subpopulation makes up 6.8%-9.4% or 6.9%-8.7% of the population of FcRn antagonist molecules.

In some embodiments, the sixth subpopulation makes up less than 17%, less than 16%, less than 15%, less than 14%, less than 13%, less than 12%, less than 11%, less than 10%, less than 9%, less than 8%, less than 7%, or less than 6% of the population of FcRn antagonist molecules. In some embodiments, the sixth subpopulation makes up about 17%, about 16%, about 15%, about 14%, about 13%, about 12%, about 11%, about 10%, about 9%, about 8%, about 7%, or about 6% of the population of FcRn antagonist molecules. In some embodiments, the sixth subpopulation makes up 17%, 16%, 15%, 14%, 13%, 12%, 11%, 10%, 9%, 8%, 7%, or 6% of the population of FcRn antagonist molecules. In some embodiments, the sixth subpopulation makes up 7%-17%, 10%-15%, or 11%-12% of the population of FcRn antagonist molecules. In some embodiments, the sixth subpopulation makes up 7.0%-14.0% or 10.0%-14.4% of the population of FcRn antagonist molecules.

In some embodiments, the seventh subpopulation makes up less than 6.0%, less than 5.5%, less than 5.0%, less than 4.5%, less than 4.0%, less than 3.5%, less than 3.0%, less than 2.5%, less than 2.0%, less than 1.5%, less than 1%, or less than 0.5% of the population of FcRn antagonist molecules. In some embodiments, the seventh subpopulation makes up about 6.0%, about 5.5%, about 5.0%, about 4.5%, about 4.0%, about 3.5%, about 3.0%, about 2.5%, about 2.0%, about 1.5%, about 1%, or about 0.5% of the population of FcRn antagonist molecules. In some embodiments, the seventh subpopulation makes up 6.0%, 5.5%, 5.0%, 4.5%, 4.0%, 3.5%, 3.0%, 2.5%, 2.0%, 1.5%, 1%, or 0.5% of the population of FcRn antagonist molecules. In some embodiments, the seventh subpopulation makes up 0.5%-5.5%, 1.0%-3.0%, or 1.5%-2.5% of the population of FcRn antagonist molecules. In some embodiments, the seventh subpopulation makes up 1.5%-5.5% or 1.4%-4.9% of the population of FcRn antagonist molecules.

In some embodiments, the eighth subpopulation makes up less than 7.5%, less than 7.0%, less than 6.5%, less than 6.0%, less than 5.5%, less than 5.0%, less than 4.5%, less than 4.0%, less than 3.5%, less than 3.0%, or less than 2.5% of the population of FcRn antagonist molecules. In some embodiments, the eighth subpopulation makes up about 7.5%, about 7.0%, about 6.5%, about 6.0%, about 5.5%, about 5.0%, about 4.5%, about 4.0%, about 3.5%, about 3.0%, or about 2.5% of the population of FcRn antagonist molecules. In some embodiments, the eighth subpopulation makes up 7.5%, 7.0%, 6.5%, 6.0%, 5.5%, 5.0%, 4.5%, 4.0%, 3.5%, 3.0%, or 2.5% of the population of FcRn antagonist molecules. In some embodiments, the eighth subpopulation makes up 2.5%-7.5%, 3.0%-5.0%, or 3.5%-4.5% of the population of FcRn antagonist molecules. In some embodiments, the eighth subpopulation makes up 2.9%-7.4% or 3.0%-6.3% of the population of FcRn antagonist molecules.

In some embodiments, the ninth subpopulation makes up less than 3.5%, less than 3.0%, less than 2.5%, less than 2.0%, less than 1.5%, less than 1%, or less than 0.5% of the population of FcRn antagonist molecules. In some embodiments, the ninth subpopulation makes up about 3.5%, about 3.0%, about 2.5%, about 2.0%, about 1.5%, about 1%, or about 0.5% of the population of FcRn antagonist molecules. In some embodiments, the ninth subpopulation makes up 3.5%, 3.0%, 2.5%, 2.0%, 1.5%, 1%, or 0.5% of the population of FcRn antagonist molecules. In some embodiments, the ninth subpopulation makes up 0.5%-3.5%, 1.5%-2.0%, or 1.0%-1.5% of the population of FcRn antagonist molecules. In some embodiments, the ninth subpopulation makes up 0.4%-3.2% or 0.5%-2.6% of the population of FcRn antagonist molecules.

In some embodiments, the tenth subpopulation makes up less than 2.0%, less than 1.5%, less than 1%, or less than 0.5% of the population of FcRn antagonist molecules. In some embodiments, the tenth subpopulation makes up about 2.0%, about 1.5%, about 1%, or about 0.5% of the population of FcRn antagonist molecules. In some embodiments, the tenth subpopulation makes up 2.0%, 1.5%, 1%, or 0.5% of the population of FcRn antagonist molecules. In some embodiments, the tenth subpopulation makes up 0.5%-2.0%, 0.5%-1.5%, or 1.0%-1.5% of the population of FcRn antagonist molecules.

In some embodiments, the eleventh subpopulation makes up less than 2.0%, less than 1.5%, less than 1%, or less than 0.5% of the population of FcRn antagonist molecules. In some embodiments, the eleventh subpopulation makes up about 2.0%, about 1.5%, about 1%, or about 0.5% of the population of FcRn antagonist molecules. In some embodiments, the eleventh subpopulation makes up 2.0%, 1.5%, 1%, or 0.5% of the population of FcRn antagonist molecules. In some embodiments, the eleventh subpopulation makes up 0.5%-2.0%, 0.5%-1.5%, or 1.0%-1.5% of the population of FcRn antagonist molecules.

In some embodiments, the population of FcRn antagonist molecules comprises one or more of the FcRn antagonists described herein. In some embodiments, the FcRn antagonist is any of those described in U.S. Patent Application No. 63/383,599, filed on Nov. 14, 2022, incorporated herein by reference in its entirety. In some embodiments, the FcRn antagonist is a population of FcRn antagonists as described in U.S. Patent Application No. 63/383,599, filed on Nov. 14, 2022, incorporated herein by reference in its entirety.

In an embodiment, the FcRn antagonist is efgartigimod (CAS Registry No. 1821402-21-4). Efgartigimod is described in further detail herein below. The term “efgartigimod” as used herein is interchangeable with “efgartigimod alfa”. In some embodiments, efgartigimod is efgartigimod alfa-fcab.

In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161).

In an embodiment, an antibody that binds specifically to FcRn and inhibits the binding of the Fc region of immunoglobulin to FcRn is nipocalimab, also known as M281. Nipocalimab is a full-length “Fc dead” IgG1 monoclonal antibody. Nipocalimab has been administered as an intravenous infusion in Phase 2 clinical trials for the treatment of myasthenia gravis (MG), warm autoimmune hemolytic anemia (WAIHA), and hemolytic disease of fetus and newborn (HDFN). Nipocalimab comprises the light chain (SEQ ID NO: 23) and heavy chain (SEQ ID NO: 24) sequences set forth in Table 3 below:

TABLE 3
Heavy chain and light chain sequences of nipocalimab

| SEQ ID NO: | Amino Acid Sequence |
|---|---|
| 23 | QSALTQPASVSGSPGQSITISCTGTGSDVGSYNLVSWYQQHPGKAP |
| | KLMIYGDSERPSGVSNRFSGSKSGNTASLTISGLQAEDEADYYCSSY |
| | AGSGIYVFGTGTKVTVLGQPKAAPSVTLFPPSSEELQANKATLVCLI |
| | SDFYPGAVTVAWKADSSPVKAGVETTTPSKQSNNKYAASSYLSLT |
| | PEQWKSHKSYSCQVTHEGSTVEKTVAPTECS |
| 24 | EVQLLESGGGLVQPGGSLRLSCAASGFTFSTYAMGWVRQAPGKGL |
| | EWVSSIGASGSQTRYADSVKGRFTISRDNSKNTLYLQMNSLRAEDT |
| | AVYYCARLAIGDSYWGQGTMVTVSSASTKGPSVFPLAPSSKSTSGG |
| | TAALGCLVKDYFPEPVTVSWNSGALTSGVHTFPAVLQSSGLYSLSS |
| | VVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFN |
| | WYVDGVEVHNAKTKPREEQYASTYRVVSVLTVLHQDWLNGKEY |
| | KCKVSNKALPAPIEKTISKAKGQPREPQVYTLPPSREEMTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPG |

In an embodiment, an antibody that binds specifically to FcRn and inhibits the binding of the Fe region of immunoglobulin to FeRn is rozanolixizumab, also known as UCB 7665. Rozanolixizumab is a full-length humanized IgG4 monoclonal antibody. Rozanolixizumab has been administered as a subcutaneous infusion in ongoing clinical trials for MG, immune thrombocytopenia (ITP), and CIDP. Rozanolixizumab comprises the light chain (SEQ ID NO: 25) and heavy chain (SEQ ID NO: 26) sequences set forth in Table 4 below:

TABLE 4
Heavy chain and light chain sequences of rozanolixizumab

| SEQ ID NO: | Amino Acid Sequence |
|---|---|
| 25 | DIQMTQSPSSLSASVGDRVTITCKSSQSLVGASGKTYLYWLFQKPG |
| | KAPKRLIYLVSTLDSGIPSRFSGSGSGTEFTLTISSLQPEDFATYYCLQ |
| | GTHFPHTFGQGTKLEIKRTVAAPSVFIFPPSDEQLKSGTASVVCLLN |
| | NFYPREAKVQWKVDNALQSGNSQESVTEQDSKDSTYSLSSTLTLS |
| | KADYEKHKVYACEVTHQGLSSPVTKSFNRGEC |
| 26 | EVPLVESGGGLVQPGGSLRLSCAVSGFTFSNYGMVWVRQAPGKGL |
| | EWVAYIDSDGDNTYYRDSVKGRFTISRDNAKSSLYLQMNSLRAED |
| | TAVYYCTTGIVRPFLYWGQGTLVTVSSASTKGPSVFPLAPCSRSTSE |
| | STAALGCLVKDYFPEPVTVSWNSGALTSGVHTFPAVLQSSGLYSLS |
| | SVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCK |
| | VSNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMHEALHNHYTQKSLSLSLGK |

In an embodiment, an antibody that binds specifically to FcRn and inhibits the binding of the Fc region of immunoglobulin to FcRn is orilanolimab, also known as SYNT001. Orilanolimab is another full-length humanized IgG4 monoclonal antibody. Orilanolimab has been administered as an intravenous infusion in Phase 2 clinical trials for treatment of WAIHA. Orilanolimab comprises the light chain (SEQ ID NO: 27) and heavy chain (SEQ ID NO: 28) sequences set forth in Table 5 below:

TABLE 5
Heavy chain and light chain sequences of orilanolimab

| SEQ ID NO: | Amino Acid Sequence |
|---|---|
| 27 | DIQMTQSPSSLSASVGDRVTITCKASDHINNWLAWYQQKPGQAPR |
| | LLISGATSLETGVPSRFSGSGTGKDYTLTISSLQPEDFATYYCQQYW |
| | STPYTFGGGTKVEIKRTVAAPSVFIFPPSDEQLKSGTASVVCLLNNF |
| | YPREAKVQWKVDNALQSGNSQESVTEQDSKDSTYSLSSTLTLSKA |
| | DYEKHKVYACEVTHQGLSSPVTKSFNRGEC |
| 28 | QVQLVQSGAELKKPGASVKLSCKASGYTFTSYGISWVKQATGQGL |
| | EWIGEIYPRSGNTYYNEKFKGRATLTADKSTSTAYMELRSLRSEDS |
| | AVYFCARSTTVRPPGIWGTGTTVTVSSASTKGPSVFPLAPCSRSTSE |
| | STAALGCLVKDYFPEPVTVSWNSGALTSGVHTFPAVLQSSGLYSLS |
| | SVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCK |
| | VSNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMHEALHNHYTQKSLSLSLG |

In an embodiment, an antibody that binds specifically to FeRn and inhibits the binding of the Fe region of immunoglobulin to FeRn is batoclimab, also known as IMVT1401/RVT1401/HBM9161. Batoclimab is another full-length “Fc dead” IgG1 monoclonal antibody. Batoclimab has been administered as a subcutaneous injection in ongoing Phase 2 clinical trials for treatment of MG and Graves' ophthalmopathy. Batoclimab comprises the light chain (SEQ ID NO: 29) and heavy chain (SEQ ID NO: 30) sequences set forth in Table 6 below:

TABLE 6
Heavy chain and light chain sequences of batoclimab

| SEQ ID NO: | Amino Acid Sequence |
|---|---|
| 29 | SYVLTQSPSVSVAPGQTARITCGGNNIGSKSVHWYQQKPGQAPVL |
| | VVYDDSDRPSGIPERFSASNSGNTATLTISRVEAGDEADYYCQVWD |
| | SSSDHVVFGGGTKLTVLGQPKAAPSVTLFPPSSEELQANKATLVCLI |
| | SDFYPGAVTVAWKADSSPVKAGVETTTPSKQSNNKYAASSYLSLT |
| | PEQWKSHRSYSCQVTHEGSTVEKTVAPTECS |
| 30 | QLLLQESGPGLVKPSETLSLTCTVSGGSLSSSFSYWVWIRQPPGKGL |
| | EWIGTIYYSGNTYYNPSLKSRLTISVDTSKNHFSLKLSSVTAADTAV |
| | YYCARRAGILTGYLDSWGQGTLVTVSSASTKGPSVFPLAPSSKSTS |
| | GGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCP |
| | PCPAPEAAGGPSVFLFPPKPKDTLMISRTPEVTCVVVDVSHEDPEVK |
| | FNWYVDGVEVHNAKTKPREEQYNSTYRVVSVLTVLHQDWLNGK |
| | EYKCKVSNKALPAPIEKTISKAKGQPREPQVYTLPPSREEMTKNQV |
| | SLTCLVKGFYPSDIAVEWESNGQPENNYKTTPPVLDSDGSFFLYSK |
| | LTVDKSRWQQGNVFSCSVMHEALHNHYTQKSLSLSPG |

In some embodiments, anti-FcRn antibodies for use according to the methods and uses described herein are any of the anti-FcRn antibodies described in International patent application no. WO2015167293A1, the contents of which are incorporated herein in their entirety. In an embodiment, an antibody that binds specifically to FcRn and inhibits the binding of the Fc region of immunoglobulin to FcRn is IMVT-1402 (Immunovant).

### C. Pharmaceutical Compositions

The instant disclosure provides pharmaceutical compositions comprising an FcRn antagonist for use in methods of treating CIDP. In certain embodiments, these compositions comprise or consist of a variant Fc region, or FcRn binding fragment thereof, that binds specifically to FcRn, particularly human FcRn, with increased affinity and reduced pH dependence relative to a native Fc region. In other embodiments, the FcRn antagonist composition is an antibody or antigen-binding fragment thereof that binds specifically to FcRn via its antigen binding domain and inhibits the binding of Fc region of immunoglobulin to FcRn. In general, these FcRn antagonists inhibit the binding of Fc-containing agents (e.g., antibodies and immunoadhesins) to FcRn in vivo, which results in an increased rate of degradation of the Fc-containing agents and, concomitantly, a reduced serum level of these agents.

In an embodiment, the FcRn antagonist is efgartigimod, or a biosimilar version thereof. Efgartigimod (ARGX-113) is a modified human immunoglobulin (Ig) gamma (IgG) 1-derived Fc of the za allotype that binds with nanomolar affinity to human FcRn. Efgartigimod encompasses the IgG1 Fc region (encompassing residues of SEQ ID NO: 2) and has been engineered using ABDEG™ technology to increase its affinity for FcRn at both physiological and acidic pH, see Vaccaro C et al., Nat Biotechnol. 2005; 23(10):1283. See also U.S. Pat. No. 10,316,073, the contents of which are incorporated by reference herein in their entirety. The increased affinity for FcRn of efgartigimod at both acidic and physiological pH results in a blockage of FcRn-mediated recycling of IgGs.

Efgartigimod has a molecular weight of about 54 kDa, which is about one-third the molecular weight of full-length IgG (MW ca. 150 kDa). Thus, 10 mg efgartigimod is about 185 nmol, such that a dose of 10 mg efgartigimod/kg body weight corresponds to about 185 nmol efgartigimod/kg body weight, and a dose of 25 mg efgartigimod/kg of body weight corresponds to about 462.5 nmol efgartigimod/kg body weight. In contrast, a dose of 10 mg full-length IgG antibody/kg body weight corresponds to about 67 nmol/kg body weight. Furthermore, a 1000 mg fixed dose of efgartigimod corresponds to a fixed dose of about 18,500 nmol of efgartigimod while a 2000 mg fixed dose of efgartigimod corresponds to a fixed dose of about 37,000 nmol of efgartigimod.

Due to its increased affinity for FcRn at both acidic and neutral pH, efgartigimod blocks the FcRn/IgG complex from forming, which results in degradation of endogenous IgGs, including autoantibodies that cause IgG-mediated autoimmune diseases. This blocking of FcRn by efgartigimod results in a rapid and profound reduction in autoantibody levels, which underlies the therapeutic strategy for the treatment of autoimmune indications where IgG autoantibodies are expected to have a central role in the disease pathology.

Efgartigimod is a prescription medicine registered as VYVGART®, which is approved in the United States Europe, United Kingdom, China, Canada, and Israel for the treatment of adults with generalized myasthenia gravis (gMG) who are anti-acetylcholine receptor (AChR) antibody positive, and in Japan for the treatment of adults with gMG who do not have sufficient response to steroids or non-steroidal immunosuppressive therapies (ISTs). Efgartigimod is also approved in Japan for the treatment of chronic ITP. Efgartigimod is under development for both the intravenous (IV) and subcutaneous (SC) administration route in multiple indications.

For SC administration, in certain embodiments efgartigimod may be administered alone. Alternatively, for SC administration, in certain embodiments efgartigimod may be administered co-formulated with hyaluronidase, for example, in particular, rHuPH20 (recombinant Human PH20). The co-formulated material will allow dosing of higher volumes.

rHuPH20 is the active ingredient of Halozyme's commercial product HYLENEX® recombinant (hyaluronidase human injection), referred to as HYLENEX®, which was approved by FDA for marketed use in the U.S. in December 2005. HYLENEX® is a tissue permeability modifier indicated as an adjuvant in SC fluid administration for achieving hydration, to increase the dispersion and absorption of other injected drugs, and in SC urography, for improving resorption of radiopaque agents.

rHuPH20 is a recombinant enzyme human hyaluronidase produced by genetically engineered Chinese hamster ovary (CHO) cells containing a deoxyribonucleic plasmid encoding a soluble fragment of human hyaluronidase (posterior head protein 20 [PH20]).

The HZ202 rHuPH20 DS is currently registered in HYLENEX® and other biologic drug products co-formulated with rHuPH20 DS. As such, in certain embodiments HZ202 rHuPH20 DS is used in the efgartigimod/rHuPH20 co-formulated product for SC administration (i.e., efgartigimod PH20 SC).

Efgartigimod PH20 SC is currently registered as VYVGART® HYTRULO, which was approved by the U.S. FDA in 2023 for the treatment of gMG in adult patients who are AChR antibody positive. VYVGART® HYTRULO contains 1,008 mg efgartigimod alfa and 11,200 units hyaluronidase per 5.6 mL (180 mg/2,000 units per mL) in a single-dose vial. In certain embodiments, efgartigimod PH20 SC (also referred to as efgartigimod PH20) is used in the methods described herein.

Provided in the co-formulations, combinations, uses and methods herein are soluble hyaluronidases. Soluble hyaluronidases include any that, upon expression, are secreted from a cell and exist in soluble form. Such soluble hyaluronidases include, but are not limited to, bacterial soluble hyaluronidases, non-human soluble hyaluronidases, such as bovine PH20 and ovine PH20, human soluble PH20, and variants thereof. Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g., DG44 CHO cells).

In some embodiments, rHuPH20 refers to the composition produced upon expression in a cell, such as a CHO cell, of nucleic acid encoding residues 36-482 of SEQ ID NO: 32, generally linked to the native or a heterologous signal sequence (residues 1-35 of SEQ ID NO: 32). rHuPH20 is produced by expression of a nucleic acid molecule, such as encoding amino acids 1-482 (set forth in SEQ ID NO: 32) in a mammalian cell. Translational processing removes the 35 amino acid signal sequence. As produced in the culture medium there is heterogeneity at the C-terminus such that the product, designated rHuPH20, includes a mixture of species that can include any one or more of the polypeptides 36-480, 36-481, and 36-482 of SEQ ID NO: 32, and some shorter polypeptides, in various abundance. Typically, rHuPH20 is produced in cells that facilitate correct N-glycosylation to retain activity, such as CHO cells (e.g., DG44 CHO cells). In some embodiments, one of the most abundant species is the 446 amino acid polypeptide corresponding to residues 36-481 of SEQ ID NO: 32. In some embodiments, rHuPH20 refers to polypeptides that are soluble or secreted upon expression in a mammalian cell and have at least 90%, 91%, 92%, 93%, 94%, 95%, 96%, 97%, 98%, 99%, or more sequence identity with residues 36-482 of SEQ ID NO: 32. In some embodiments, the rHuPH20 is the 447 amino acid polypeptide of SEQ ID NO: 33.

TABLE 7
Exemplary hyaluronidase sequences

| SEQ ID NO: | Amino Acid Sequence |
|---|---|
| 32 | MGVLKFKHIFFRSFVKSSGVSQIVFTFLLIPCCLTLNFRAPPVIPNVPF |
| | LWAWNAPSEFCLGKFDEPLDMSLFSFIGSPRINATGQGVTIFYVDRL |
| | GYYPYIDSITGVTVNGGIPQKISLQDHLDKAKKDITFYMPVDNLGM |
| | AVIDWEEWRPTWARNWKPKDVYKNRSIELVQQQNVQLSLTEATE |
| | KAKQEFEKAGKDFLVETIKLGKLLRPNHLWGYYLFPDCYNHHYKK |
| | PGYNGSCFNVEIKRNDDLSWLWNESTALYPSIYLNTQQSPVAATLY |
| | VRNRVREAIRVSKIPDAKSPLPVFAYTRIVFTDQVLKFLSQDELVYT |
| | FGETVALGASGIVIWGTLSIMRSMKSCLLLDNYMETILNPYIINVTL |
| | AAKMCSQVLCQEQGVCIRKNWNSSDYLHLNPDNFAIQLEKGGKFT |
| | VRGKPTLEDLEQFSEKFYCSCYSTLSCKEKADVKDTDAVDVCIADG |
| | VCIDAFLKPPMETEEPQIFYNASPSTLSATMFIVSILFLIISSVASL |
| 33 | LNFRAPPVIPNVPFLWAWNAPSEFCLGKFDEPLDMSLFSFIGSPRIN |
| | ATGQGVTIFYVDRLGYYPYIDSITGVTVNGGIPQKISLQDHLDKAK |
| | KDITFYMPVDNLGMAVIDWEEWRPTWARNWKPKDVYKNRSIELV |
| | QQQNVQLSLTEATEKAKQEFEKAGKDFLVETIKLGKLLRPNHLWG |
| | YYLFPDCYNHHYKKPGYNGSCPNVEIKRNDDLSWLWNESTALYPS |
| | IYLNTQQSPVAATLYVRNRVREAIRVSKIPDAKSPLPVFAYTRIVFT |
| | DQVLKFLSQDELVYTFGETVALGASGIVIWGTLSIMRSMKSCLLLD |
| | NYMETILNPYIINVTLAAKMCSQVLCQEQGVCIRKNWNSSDYLHL |
| | NPDNFAIQLEKGGKFTVRGKPTLEDLEQFSEKFYCSCYSTLSCKEK |
| | ADVKDTDAVDVCIADGVCIDAFLKPPMETEEPQIFY |

SC injection volumes are typically limited to 2.5 mL due to concerns regarding injection pain associated with larger volumes. It has been demonstrated that rHuPH20 offers a solution to the volume limitation associated with fast SC injections. rHuPH20 acts locally and transiently to depolymerize hyaluronan, a gel-like substance found in the subcutaneous layer of the skin. This results in decreased resistance to fluid flow and may increase dispersion and absorption of injected medicines and fluids, allowing for a larger volume to be injected with limited swelling or pain. It has been shown that rHuPH20 allows for the fast absorption of a relatively large volume (10 mL) when administered SC (Shpilberg O et al., 2013). Very little injection site swelling was observed when 10 mL of IgG solution was administered SC using rHuPH20 at 2000 U/mL, whereas a large injection site swelling was observed when 10 mL of IgG solution was injected without rHuPH20 (Shpilberg O et al., 2013).

rHuPH20 is transiently acting and is not systematically absorbed. It has been demonstrated to exert no long-term local effects. rHuPH20 has a half-life in the skin of less than 30 minutes. Hyaluronan levels in subcutaneous tissues return to normal within 24 to 48 hours because of the rapid natural turnover of hyaluronan.

rHuPH20 is approved for SC administration in co-formulations with other active ingredients (RITUXAN HYCELA™/MABTHERA™ SC [rituximab] for Non-Hodgkin's lymphoma (NHL) and chronic lymphocytic leukemia (CLL) and HERCEPTIN HYLECTA™/HERCEPTIN™ SC [trastuzumab]) in the U.S. and Europe with an enzyme concentration of 2000 U/mL and an injectable volume that ranges from 5 to 13.4 mL.

In some embodiments, the pharmaceutical formulation comprises an FcRn antagonist in an amount from about 20 mg to about 20,000 mg. In some embodiments, the pharmaceutical formulation comprises an FcRn antagonist in an amount from about 200 mg to about 20,000 mg. In some embodiments, the pharmaceutical formulation comprises an FcRn antagonist in an amount from about 300 mg to about 6000 mg. In some embodiments, the pharmaceutical formulation comprises an FcRn antagonist in an amount from about 750 mg to about 3000 mg. In some embodiments, the pharmaceutical formulation comprises an FcRn antagonist in an amount from about 1000 mg to about 2500 mg. In some embodiments, the pharmaceutical formulation comprises an FcRn antagonist in an amount from about 1000 mg to about 2000 mg.

In some embodiments, the pharmaceutical formulation comprises an FcRn antagonist in an amount from 20 mg to 20,000 mg. In some embodiments, the pharmaceutical formulation comprises an FcRn antagonist in an amount from 200 mg to 20,000 mg. In some embodiments, the pharmaceutical formulation comprises an FcRn antagonist in an amount from 300 mg to 6000 mg. In some embodiments, the pharmaceutical formulation comprises an FcRn antagonist in an amount from 750 mg to 3000 mg. In some embodiments, the pharmaceutical formulation comprises an FcRn antagonist in an amount from 1000 mg to 2500 mg. In some embodiments, the pharmaceutical formulation comprises an FcRn antagonist in an amount from 1000 mg to 2000 mg.

In some embodiments, the pharmaceutical formulation comprises about 1000 mg or about 2000 mg of an FcRn antagonist. In some embodiments, the pharmaceutical formulation comprises 1000 mg or 2000 mg of an FcRn antagonist. In some embodiments, the FcRn antagonist is efgartigimod.

In some embodiments, the pharmaceutical formulation comprises efgartigimod in an amount from about 800 mg to about 1200 mg. In some embodiments, the pharmaceutical formulation comprises about 1000 mg efgartigimod. In some embodiments, the pharmaceutical formulation comprises 1000 mg efgartigimod.

In some embodiments, the pharmaceutical formulation comprises from about 10 mg/mL to about 200 mg/mL efgartigimod. In some embodiments, the pharmaceutical formulation comprises from 10 mg/mL to 200 mg/mL efgartigimod.

In some embodiments, the pharmaceutical formulation comprises about 20 mg/mL efgartigimod. In some embodiments, the pharmaceutical formulation comprises 20 mg/mL efgartigimod.

In some embodiments, the pharmaceutical formulation comprises about 180 mg/mL efgartigimod. In some embodiments, the pharmaceutical formulation comprises 180 mg/mL efgartigimod.

In some embodiments, the pharmaceutical formulation further comprises hyaluronidase. In some embodiments, the hyaluronidase is recombinant human hyaluronidase PH20 (rHuPH20).

The hyaluronidase can be present in the pharmaceutical formulation in any suitable amount. In an embodiment, the amount of hyaluronidase is from about 1000 U/mL to about 3000 U/mL. In an embodiment, the amount of hyaluronidase is about 1000 U/mL, about 1500 U/mL, about 2000 U/mL, about 2500 U/mL, or about 3000 U/mL. In an embodiment, the amount of hyaluronidase is 2000 U/mL.

In some embodiments, the rHuPH20 is present in the pharmaceutical formulation in an amount of about 11,000 U or about 22,000 U. In some embodiments, the rHuPH20 is present in the pharmaceutical formulation in an amount of 11,000 U or 22,000 U. In some embodiments the rHuPH20 is present in the pharmaceutical formulation in an amount of about 11,200 U. In some embodiments the rHuPH20 is present in the pharmaceutical formulation in an amount of 11,200 U.

In some embodiments, the pharmaceutical formulation comprises at least about 5 U to at least about 100,000 U of an endoglycosidase hydrolase enzyme. In some aspects, the pharmaceutical formulation comprises at least about 5 U, at least about 10 U, at least about 20 U, at least about 30 U, at least about 40 U, at least about 50 U, at least about 75 U, at least about 100 U, at least about 200 U, at least about 300 U, at least about 400 U, at least about 500 U, at least about 750 U, at least about 1000 U, at least about 2000 U, at least about 3000 U, at least about 4000 U, at least about 5000 U, at least about 6000 U, at least about 7000 U, at least about 8000 U, at least about 9000 U, at least about 10,000 U, at least about 20,000 U, at least about 30,000 U, at least about 40,000 U, at least about 50,000 U, at least about 60,000 U, at least about 70,000 U, at least about 80,000 U, at least about 90,000 U, or at least about 100,000 U of an endoglycosidase hydrolase enzyme.

In some embodiments, the pharmaceutical formulation comprises about 20,000 U of an endoglycosidase hydrolase enzyme. In some embodiments, the pharmaceutical formulation comprises at least about 500 U/mL to at least about 5000 U/mL of an endoglycosidase hydrolase enzyme. In some embodiments, the pharmaceutical formulation comprises at least about 1500 U/mL, at least about 1600 U/mL, at least about 1700 U/mL, at least about 1800 U/mL, at least about 1900 U/mL, at least about 2000 U/mL, at least about 2100 U/mL, at least about 2200 U/mL, at least about 2300 U/mL, at least about 2400 μM, at least about 2500 μM, at least about 3000 μM, at least about 3500 μM, at least about 4000 μM, at least about 4500 U/mL, or at least about 5000 U/mL of an endoglycosidase hydrolase enzyme. In some embodiments, the pharmaceutical formulation comprises about 2000 U/mL of an endoglycosidase hydrolase enzyme.

In some embodiments, the endoglycosidase hydrolase enzyme cleaves hyaluronic acid at a hexosaminidic β (1-4) or (1-3) linkage. In some embodiments, the endoglycosidase hydrolase enzyme comprises a catalytic domain of hyaluronidase PH-20 (HuPH20), HYAL1, HYAL2, HYAL3, HYAL4, or HYALPS1. In some embodiments, the endoglycosidase hydrolase enzyme comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to amino acids 36-490 of SEQ ID NO: 32. In some embodiments, the endoglycosidase hydrolase enzyme comprises a hyaluronidase. In some embodiments, the endoglycosidase hydrolase enzyme comprises a hyaluronidase selected from the group consisting of HuPH20, HYAL1, HYAL2, HYAL3, HYAL4, any variant, and any isoform thereof. In some embodiments, the endoglycosidase hydrolase enzyme comprises rHuPH20 or a fragment thereof.

In some embodiments, the endoglycosidase hydrolase enzyme comprises a modified hyaluronidase comprising one or more amino acid substitutions relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase comprising one or more amino acid substitution in an alpha-helix region relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase comprising one or more amino acid substitution in linker region relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase, wherein one or more N-terminal and/or C-terminal amino acids are deleted relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified rHuPH20, wherein the modified rHuPH20 comprises: i. one or more amino acid substitution in an alpha-helix region, a linker region, or both an alpha-helix region and a linker region relative to wild-type rHuPH20; ii. deletion of one or more N-terminal amino acid, one or more C-terminal amino acid, or one or more N-terminal amino acid and one or more C-terminal amino acid relative to wild-type rHuPH20; or iii. both (i) and (ii).

“Hyaluronidase,” as used herein, refers to an enzyme capable of catalyzing the cleavage of hyaluronan. Hyaluronan is a repeating polymer of N-acetyl-glucosamine and glucuronic acid, which is present in the subcutaneous space and contributes to the soluble gel-like component of the extracellular matrix of the skin and is restored by rapid turnover (resynthesis). In some embodiments, the hyaluronidase comprises rHuPH20, which is a glycosylated 447-amino acid single chain polypeptide that depolymerizes hyaluronan in the subcutaneous space locally at the site of injection in the skin. Depolymerization of hyaluronan by hyaluronidase is accomplished by hydrolysis of the polysaccharide polymer. Depolymerization of hyaluronan results in a transient reduction in the viscosity of the gel-like phase of the extracellular matrix and increased hydraulic conductance that facilitates the dispersion and absorption of the coadministered therapeutic agent. Thus, a hyaluronidase, e.g., rHuPH20, can improve the speed and ease of subcutaneous delivery of injectable biologics and drugs by acting as a permeation enhancer. In certain embodiments, the hyaluronidase comprises ENHANZE™.

In some embodiments, the pharmaceutical formulation comprises about 180 mg/mL of an FcRn antagonist, about 2,000 U/mL rHuPH20, about 1.4 mg/mL L-histidine, about 2.2 mg/mL L-histidine hydrochloride monohydrate, about 1.5 mg/mL L-methionine, about 0.4 mg/mL polysorbate 20, about 5.8 mg/mL sodium chloride, and about 20.5 mg/mL sucrose, at a pH of about 6.0. In some embodiments, the FcRn antagonist is efgartigimod, or a biosimilar version thereof.

In some embodiments, the pharmaceutical formulation comprises 180 mg/mL of an FcRn antagonist, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, and 20.5 mg/mL sucrose, at a pH of about 6.0. In some embodiments, the FcRn antagonist is efgartigimod, or a biosimilar version thereof.

In some embodiments, the pharmaceutical formulation comprises about 180 mg/mL efgartigimod, about 2,000 U/mL rHuPH20, about 1.4 mg/mL L-histidine, about 2.2 mg/mL L-histidine hydrochloride monohydrate, about 1.5 mg/mL L-methionine, about 0.4 mg/mL polysorbate 20, about 5.8 mg/mL sodium chloride, and about 20.5 mg/mL sucrose, at a pH of about 6.0.

In some embodiments, the pharmaceutical formulation comprises 180 mg/mL efgartigimod, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, and 20.5 mg/mL sucrose, at a pH of about 6.0.

In some embodiments, the pharmaceutical formulation comprises about 180 mg/mL of an FcRn antagonist, about 2,000 U/mL rHuPH20, about 20 mM L-histidine/L-histidine HCl, about 10 mM L-methionine, about 0.04% (w/v) polysorbate 20, about 100 mM sodium chloride, and about 60 mM sucrose, at a pH of about 6.0. In some embodiments, the FcRn antagonist is efgartigimod, or a biosimilar version thereof.

In some embodiments, the pharmaceutical formulation comprises 180 mg/mL of an FcRn antagonist, 2,000 U/mL rHuPH20, 20 mM L-histidine/L-histidine HCl, 10 mM L-methionine, 0.04% (w/v) polysorbate 20, 100 mM sodium chloride, and 60 mM sucrose, at a pH of about 6.0. In some embodiments, the FcRn antagonist is efgartigimod, or a biosimilar version thereof.

In some embodiments, the pharmaceutical formulation comprises about 180 mg/mL efgartigimod, about 2,000 U/mL rHuPH20, about 20 mM L-histidine/L-histidine HCl, about 10 mM L-methionine, about 0.04% (w/v) polysorbate 20, about 100 mM sodium chloride, and about 60 mM sucrose, at a pH of about 6.0.

In some embodiments, the pharmaceutical formulation comprises 180 mg/mL efgartigimod, 2,000 U/mL rHuPH20, 20 mM L-histidine/L-histidine HCl, 10 mM L-methionine, 0.04% (w/v) polysorbate 20, 100 mM sodium chloride, and 60 mM sucrose, at a pH of about 6.0.

In any of the above embodiments, the pharmaceutical formulation may be a unit dosage form.

In an embodiment, the unit dosage form comprises the FcRn antagonist as a dry formulation for dissolution such as a lyophilized powder, freeze-dried powder, or water-free concentrate. In an embodiment, the dry formulation is comprised in a hermetically sealed container such as a vial, an ampoule, or a sachet.

In an embodiment, the unit dosage form comprises the FcRn antagonist as a liquid formulation, e.g., injection or infusion solution. In an embodiment, the liquid formulation is comprised in a hermetically sealed container such as a vial, a sachet, a pre-filled syringe, a pre-filled autoinjector, or a cartridge for a reusable syringe or applicator. In an embodiment, the liquid formulation is comprised in a single-dose vial.

In an embodiment, the unit dosage per vial may contain 0.5 mL, 1 mL, 2 mL, 3 mL, 4 mL, 5 mL, 6 mL, 7 mL, 8 mL, 9 mL, 10 mL, 15 mL, or 20 mL of an FcRn antagonist ranging from about 500 to about 2500 mg or from about 1000 mg to about 2000 mg. In some embodiments, the unit dosage per vial contains 5.6 mL of an FcRn antagonist in an amount of 1008 mg. In some embodiments, the unit dosage per vial contains 5.6 mL of an FcRn antagonist in an amount of 1008 mg and a hyaluronidase in an amount of 11,200 U. In an embodiment, these preparations can be adjusted to a desired concentration by adding a sterile diluent to each vial.

In some embodiments, the unit dosage form is a single-dose vial that contains at least 5.6 mL of a liquid formulation comprising an FcRn antagonist at a concentration of 180 mg/mL. In some embodiments, the single-dose vial contains 5.6 mL of a liquid formulation comprising 1,008 mg of an FcRn antagonist, and 11,200 units hyaluronidase (human recombinant). Each mL of vial solution contains 180 mg of efgartigimod alfa, 2,000 units of hyaluronidase (human recombinant) and histidine (1.4 mg), L-histidine hydrochloride monohydrate (2.2 mg), methionine (1.5 mg), polysorbate 20 (0.4 mg), sodium chloride (5.8 mg), sucrose (20.5 mg), and water for injection, USP, at a pH of 6.0. In some embodiments, the FcRn antagonist is efgartigimod, or a biosimilar version thereof.

The formulations disclosed herein include bulk drug compositions useful in the manufacture of pharmaceutical compositions (e.g., compositions that are suitable for administration to a subject or patient) which can be used in the preparation of unit dosage forms. In an embodiment, a composition of the invention is a pharmaceutical composition. Such compositions comprise a prophylactically or therapeutically effective amount of one or more prophylactic or therapeutic agents (e.g., an FcRn antagonist of the invention or other prophylactic or therapeutic agent), and a pharmaceutically acceptable carrier. In an embodiment, the pharmaceutical compositions are formulated to be suitable for intravenous administration to a subject. In an embodiment, the pharmaceutical compositions are formulated to be suitable for subcutaneous administration to a subject. In some embodiments, the pharmaceutical compositions are formulated to be suitable for subcutaneous administration to a subject as once weekly injections over approximately 30 to 90 seconds. In some embodiments, the pharmaceutical compositions are formulated for subcutaneous use with a winged infusion set.

In some embodiments, the pharmaceutical compositions described herein are contraindicated in patients with serious hypersensitivity to the FcRn antagonist (e.g., efgartigimod), to hyaluronidase, or to any of the excipients in the compositions. In some embodiments, the hypersensitivity is anaphylaxis and/or hypotension leading to syncope.

### D. Kits

The present disclosure provides kits comprising an FcRn antagonist and a label. In some embodiments, the kit comprises a pharmaceutical composition comprising an FcRn antagonist and a label. Any FcRn antagonist described herein, or pharmaceutical composition thereof, may be included in the kits provided herein. In some embodiments, the FcRn antagonist is efgartigimod, or a biosimilar version thereof.

In some embodiments, the pharmaceutical composition comprises about 180 mg/mL of an FcRn antagonist, about 2,000 U/mL rHuPH20, about 1.4 mg/mL L-histidine, about 2.2 mg/mL L-histidine hydrochloride monohydrate, about 1.5 mg/mL L-methionine, about 0.4 mg/mL polysorbate 20, about 5.8 mg/mL sodium chloride, and about 20.5 mg/mL sucrose, at a pH of about 6.0. In some embodiments, the FcRn antagonist is efgartigimod, or a biosimilar version thereof.

In some embodiments, the pharmaceutical composition comprises 180 mg/mL of an FcRn antagonist, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, and 20.5 mg/mL sucrose, at a pH of about 6.0. In some embodiments, the FcRn antagonist is efgartigimod, or a biosimilar version thereof.

In some embodiments, the pharmaceutical composition comprises about 180 mg/mL efgartigimod, about 2,000 U/mL rHuPH20, about 1.4 mg/mL L-histidine, about 2.2 mg/mL L-histidine hydrochloride monohydrate, about 1.5 mg/mL L-methionine, about 0.4 mg/mL polysorbate 20, about 5.8 mg/mL sodium chloride, and about 20.5 mg/mL sucrose, at a pH of about 6.0.

In some embodiments, the pharmaceutical composition comprises 180 mg/mL efgartigimod, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, and 20.5 mg/mL sucrose, at a pH of about 6.0.

In some embodiments, the pharmaceutical composition comprises about 180 mg/mL of an FcRn antagonist, about 2,000 U/mL rHuPH20, about 20 mM L-histidine/L-histidine HCl, about 10 mM L-methionine, about 0.04% (w/v) polysorbate 20, about 100 mM sodium chloride, and about 60 mM sucrose, at a pH of about 6.0. In some embodiments, the FcRn antagonist is efgartigimod, or a biosimilar version thereof.

In some embodiments, the pharmaceutical composition comprises 180 mg/mL of an FcRn antagonist, 2,000 U/mL rHuPH20, 20 mM L-histidine/L-histidine HCl, 10 mM L-methionine, 0.04% (w/v) polysorbate 20, 100 mM sodium chloride, and 60 mM sucrose, at a pH of about 6.0. In some embodiments, the FcRn antagonist is efgartigimod, or a biosimilar version thereof.

In some embodiments, the pharmaceutical composition comprises about 180 mg/mL efgartigimod, about 2,000 U/mL rHuPH20, about 20 mM L-histidine/L-histidine HCl, about 10 mM L-methionine, about 0.04% (w/v) polysorbate 20, about 100 mM sodium chloride, and about 60 mM sucrose, at a pH of about 6.0.

In some embodiments, the pharmaceutical composition comprises 180 mg/mL efgartigimod, 2,000 U/mL rHuPH20, 20 mM L-histidine/L-histidine HCl, 10 mM L-methionine, 0.04% (w/v) polysorbate 20, 100 mM sodium chloride, and 60 mM sucrose, at a pH of about 6.0.

In some embodiments, the kit comprises a single dose vial comprising the FcRn antagonist as a liquid formulation, and a label.

In some embodiments, the single-dose vial contains 5.6 mL of a liquid formulation comprising 1,008 mg of an FcRn antagonist, 11,200 units rHuPH20, 7.8 mg histidine, 12.3 mg L-histidine hydrochloride monohydrate, 8.4 mg methionine, 2.2 mg polysorbate 20, 32.5 mg sodium chloride, 114.8 mg sucrose, and water for injection, USP, at a pH of 6.0. In some embodiments, the FcRn antagonist is efgartigimod, or a biosimilar version thereof.

In some embodiments, the single-dose vial contains 5.6 mL of a liquid formulation comprising 1,008 mg of an FcRn antagonist and 11,200 Units of rHuPH20, and each mL of vial solution contains histidine (1.4 mg), L-histidine hydrochloride monohydrate (2.2 mg), methionine (1.5 mg), polysorbate 20 (0.4 mg), sodium chloride (5.8 mg), sucrose (20.5 mg), and water for injection, USP, at a pH of 6.0. In some embodiments, the FcRn antagonist is efgartigimod, or a biosimilar version thereof.

In some embodiments, the label identifies efgartigimod and provides instructions for its use in a patient. In some embodiments, the label includes an indication for the treatment of CIDP in adult patients. In some embodiments, the label includes data demonstrating an ECI response in 66.5% of a population of CIDP patients who received 1008 mg/11,200 units of efgartigimod PH20 once weekly. In some embodiments, the label includes data demonstrating improvement at two consecutive visits in 69% of a population of CIDP patients who received 1008 mg/11,200 units of efgartigimod PH20 once weekly up to 12 weeks. In some embodiments, the improvement is aINCAT improvement ≥1 point, I-RODS improvement ≥4 points, or mean grip strength improvement ≥8 kPa. In some embodiments, the label includes data demonstrating the median time to initial confirmed ECI in a population of CIDP patients is 43 days from first administration of efgartigimod PH20. In some embodiments, the label states that at week 4, the earliest time point for which ECI criteria could have been met, up to 40% of patients had ECI. In some embodiments, the label states that 25% of patients showed clinically relevant improvement after 9 days in at least one of the 3 parameters (aINCAT, I-RODS, or Grip Strength). In some embodiments, the label includes data demonstrating that CIDP patients who received the drug product experienced a longer time to clinical deterioration compared to the CIDP patients who received placebo, wherein the clinical deterioration is an increase of ≥1 point in aINCAT score. In some embodiments, the clinical deterioration is an increase in aINCAT score of ≥1 points during two consecutive measurements. In some embodiments, the clinical deterioration is an increase in aINCAT score of ≥2 points. In some embodiments, the longer time to clinical deterioration is demonstrated by a hazard ratio of 0.394. In some embodiments, the longer time to clinical deterioration is statistically significant. In some embodiments, the label states that patients who received efgartigimod PH20 experienced a longer time to clinical deterioration (i.e., increase of ≥1 point in aINCAT score) compared patients who received placebo, which was statistically significant, as demonstrated by a hazard ratio of 0.394 [95% CI (0.253; 0.614) p<0.0001].

In some embodiments, the label includes data demonstrating CIDP patients in a population of CIDP patients who received efgartigimod PH20 remained relapse-free significantly longer than CIDP patients in a population of CIDP patients who received placebo. In some embodiments, the label includes data demonstrating CIDP patients in a population of CIDP patients who received efgartigimod PH20 demonstrated a 61% risk reduction for deterioration compared to CIDP patients in a population of CIDP patients who received placebo.

In some embodiments, the label includes data indicating a 2% incidence of anti-efgartigimod alfa antibodies in a population of 117 CIDP patients following treatment with the drug product. In some embodiments, the label includes data indicating a 6% incidence of anti-efgartigimod alfa antibodies in a population of 317 CIDP patients following treatment with the drug product. In some embodiments, the label includes data indicating a 0.3% incidence of neutralizing anti-efgartigimod alfa antibodies in a population of 317 CIDP patients following treatment with the drug product.

In some embodiments, the label includes a contraindication in patients with serious hypersensitivity to efgartigimod alfa products, to hyaluronidase, or to any excipients in formulations thereof. In some embodiments, the label includes a warning for hypersensitivity reactions selected from anaphylaxis and hypotension leading to syncope. In some embodiments, the label states infusion discontinuation when anaphylaxis or hypotension leading to syncope occur during or within 1 hour of administration of the product. In some embodiments, the label includes a warning for infusion-related reactions selected from hypertension, chills, shivering, thoracic pain, abdominal pain, and back pain. In some embodiments, the label states to initiate appropriate therapy when a severe infusion-related reaction occurs during administration of the product. In some embodiments, the label states that patients may be rechallenged with close clinical observation, slower infusion rates, and pre-medications when a mild-to-moderate infusion-related reaction occurs during administration of the product. In some embodiments, the label includes a combination of any of the features described above or elsewhere herein.

### E. Methods of Treatment

### Subjects to be Treated

The present invention provides methods of treating CIDP. As explained elsewhere herein, CIDP is a heterogenous disease encompassing disorders with predominantly sensory symptoms, distal symmetric disorder or distal acquired demyelinating symmetric neuropathy (DADS), multifocal acquired demyelinating sensory and motor neuropathy (MADSAM) and CIDP with associated central nervous system (CNS) demyelination. Misdiagnosis of CIDP occurs relatively frequently.

In some embodiments, subjects to be treated in accordance with the methods described herein have been diagnosed with CIDP using the official EFNS/PNS 2010 diagnostic criteria (Van den Bergh et al., 2010). In some embodiments, subjects have been diagnosed as having definite, probable CIDP. In some embodiments, subjects have been diagnosed as having possible CIDP.

The EFNS/PNS 2010 diagnostic criteria were revised in 2021, see Van den Bergh et al., Eur J Neurol. 2021; 28: 3556-3583, and distinguish between “typical CIDP” and “CIDP variants” as defined in Table 8 below. CIDP variants were previously classed as “atypical” forms of CIDP.

TABLE 8
Clinical criteria for Typical CIDP and CIDP Variants

Typical CIDP
All the following:
Progressive or relapsing, symmetric, proximal and distal muscle
weakness of upper and lower limbs, and sensory involvement of
at least two limbs
Developing over at least 8 weeks
Absent or reduced tendon reflexes in all limbs
CIDP variants
One of the following, but otherwise as in typical CIDP (tendon
reflexes may be normal in unaffected limbs):
Distal CIDP: distal sensory loss and muscle weakness
predominantly in lower limbs
Multifocal CIDP: sensory loss and muscle weakness in a
multifocal pattern, usually asymmetric, upper limb predominant,
in more than one limb
Focal CIDP: sensory loss and muscle weakness in only one limb
Motor CIDP: motor symptoms and signs without sensory
involvement
Sensory CIDP: sensory symptoms and signs without motor
involvement

Subjects treated in accordance with the methods described herein may have typical CIDP. Typical CIDP typically begins with paraesthesia and weakness in the distal limbs as well as difficulty walking. Clinical examination shows progressive symmetric proximal and distal muscle weakness, sensory loss, and decreased or absent deep tendon reflexes.

Alternatively, subjects treated in accordance with the methods described herein may have a CIDP variant form selected from the group consisting of: Distal CIDP; Multifocal CIDP, Focal CIDP, Motor CIDP and Sensory CIDP. In certain embodiments, subjects treated in accordance with the methods described herein have a CIDP variant form selected from the group consisting of: Distal CIDP; Multifocal CIDP, Focal CIDP and Motor CIDP. These CIDP variants are described in more detail below.
Distal CIDP is also known as “Distal Acquired Demyelinating Symmetric Neuropathy” (DADS) or as “distal symmetric disorder”; this variant of CIDP presents with sensory loss in the distal upper and lower limbs as well as gait instability. Weakness may occur and is usually distally accentuated in lower more than upper limbs.
Multifocal CIDP is also known as “Multifocal Demyelinating Neuropathy with persistent conduction block”, “Lewis-Sumner syndrome” (LSS), “Multifocal Acquired Demyelinating Sensory and Motor Neuropathy” (MADSAM) or Multifocal Inflammatory Demyelinating Neuropathy; this variant of CIDP usually affects the upper limbs first and lower limbs may become involved later. Cranial nerves are more frequently involved than in other forms of CIDP.
Focal CIDP; this variant is rare and usually affects the brachial or lumbosacral plexus but can affect individual peripheral nerves.
Motor CIDP; this variant presents as relatively symmetric proximal and distal weakness but with normal sensation clinically and electrodiagnostically (in contrast to typical CIDP where sensation is abnormal.
Sensory CIDP; this variant is usually characterized by gait ataxia, impairment of vibration and position sense and changes in cutaneous sensation. Muscle weakness is not present.

CIDP can also be classified at diagnosis as “progressive” or “relapsing”. In certain embodiments, subjects treated in accordance with the methods described herein have progressive CIDP. In certain embodiments, subjects treated in accordance with the methods described herein have relapsing CIDP.

In some instances, nodal and paranodal autoantibodies have been identified in patients with CIDP. These antibodies include anti-NF155 antibodies (Cortese et al., 2020), anti-CNTN1 antibodies (Querol et al., 2013), anti-Caspr1 antibodies (Pascual-Goñi et al., 2021) and anti-NF140/186 antibodies (Delmont et al., 2017).

In some embodiments, subjects treated in accordance with the methods described herein are CIDP patients characterised by the presence of anti-NF155 antibodies. In some embodiments, subjects treated in accordance with the methods described herein are CIDP patients characterised by the presence of anti-CNTN1 antibodies. In some embodiments, subjects treated in accordance with the methods described herein are CIDP patients characterised by the presence of anti-Caspr1 antibodies. In some embodiments, subjects treated in accordance with the methods described herein are CIDP patients characterised by the presence of anti-NF140/186 antibodies.

In some embodiments, subjects treated in accordance with the methods described herein are CIDP patients characterised by the presence of anti-GM1 antibodies. In some embodiments, subjects treated in accordance with the methods described herein are CIDP patients characterised by the presence of anti-LM1 antibodies.

Subjects with CIDP treated in accordance with the methods described herein may have mild, moderate or severe CIDP.

In some embodiments, subjects treated in accordance with the methods described herein are adult human subjects. In some embodiments, subjects treated in accordance with the methods described herein are adult human subjects ranging in age from about 20 to about 82 years. In some embodiments, subjects treated in accordance with the methods described herein are adult human subjects ranging in age from 20 to 82 years. The severity of a subject's CIDP disease may be assessed using any of the assessment methods described herein including but not limited to: (i) the INCAT Disability Scale (Merkies et al., 2002); (ii) the MRC Scale (Vanhoutte et al., 2012); (iii) the I-RODS (van Nes et al., 2011); (iv) the Mean Grip Strength test (Vanhoutte et al., 2013); and (v) the TUG test.

In certain embodiments, CIDP disease severity is assessed using the INCAT Disability Scale i.e., the scale accepted by the FDA for assessing the severity of motor disability. The INCAT scale is described elsewhere herein and is a 10-point scale that covers the functionality of legs and arms. Scores for arm disability range from 0 to 5 and scores for leg disability also range from 0 to 5 (with 0 being normal and 5 being the most impaired). The total INCAT score is the sum of the arm and leg scores and ranges from 0 to 10. In certain embodiments, CIDP symptoms are assessed using the aINCAT score. In accordance with the aINCAT score, changes in the function of the arms from 0 (normal) to 1 (minor symptoms) or from 1 to 0 are not recorded as deterioration or improvement because these small changes are not considered clinically significant.

In certain embodiments, subjects treated in accordance with the methods described herein have a baseline INCAT score (i.e., prior to treatment with an FcRn antagonist) of 2 or more, optionally 3 or more, optionally 4 or more, optionally 5 or more, optionally 6 or more, optionally 7 or more, optionally 8 or more, optionally 9 or more, optionally 10. In certain embodiments, subjects have a baseline INCAT score of 2, 3, 4 or 5, wherein the disability is limited to the arms. In certain embodiments, subjects have a baseline INCAT score of 2, 3, 4 or 5 wherein the disability is limited to the legs. In certain embodiments, subjects have a baseline INCAT score of 2, 3, 4 or 5 wherein disability affects both the arms and legs. In certain embodiments, subjects have a baseline INCAT score of 5, wherein disability affects arms, legs, or both. In some embodiments, subjects have a baseline INCAT score of 3, wherein disability affects arms, legs, or both.

In certain embodiments, CIDP disease severity is assessed using the I-RODS. As described elsewhere herein, I-RODS is a 24-item scale, with each item representing a common daily activity that ranges from very difficult to do, like running or dancing, to very easy to do, like eating or reading a book. Higher scores indicate less disability. The raw scores of the I-RODS (range: 0 to 48) are typically converted to a centile metric score, ranging from 0 (most severe activity and social participation restrictions) to 100 (no activity and social participation limitations). In certain embodiments, subjects treated in accordance with the methods described herein have a baseline I-RODS centile metric score of 10 or more, optionally 20 or more, optionally 30 or more, optionally 40 or more, optionally 50 or more. In certain embodiments, subjects treated in accordance with the methods described herein have a baseline I-RODS centile metric score of 37.

In some embodiments, CIDP disease severity is assessed using a Mean Grip Strength test. Mean grip strength can be measured using a handheld Martin vigorimeter or a Jamar hand grip dynamometer. In some embodiments, subjects treated in accordance with the methods described herein have a baseline mean grip strength (as measured using the dominant hand) of ≥20 kPa, optionally ≥30 kPa. In some embodiments, subjects treated in accordance with the methods described herein have a baseline mean grip strength of 1-100 kPa, optionally 10-90 kPa, optionally 20-80 kPa, optionally 30-70 kPa, optionally 30-50 kPa.

In some embodiments, subjects treated in accordance with the methods described herein have newly-diagnosed CIDP.

In some embodiments, subjects treated in accordance with the methods described herein have CIDP that has been diagnosed about 2 years prior to treatment with the FcRn antagonist. In some embodiments, subjects treated in accordance with the methods described herein have CIDP that has been diagnosed about 2.2 years prior to treatment with the FcRn antagonist. In some embodiments, subjects treated in accordance with the methods described herein have CIDP that has been diagnosed 2 years prior to treatment with the FcRn antagonist. In some embodiments, subjects treated in accordance with the methods described herein have CIDP that has been diagnosed 2.2 years prior to treatment with the FcRn antagonist.

In some embodiments, subjects have relapsing CIDP. In some embodiments, subjects have CIDP that is unstable and refractory to treatment, e.g., characterised by a CIDP Disease Activity Score (CDAS) of 5B or 5C.

In some embodiments, subjects treated in accordance with the methods described herein are treatment-naïve. Subjects who are classed as “treatment-naïve” may have never before received any treatment for CIDP. Subjects who are classed as “treatment-naïve” may also include subjects who have previously received treatment for CIDP but have not received any treatment for CIDP in the six months prior to treatment with the FcRn antagonist as described herein.

In some embodiments, subjects treated in accordance with the methods described herein have previously received one or more treatments for CIDP, including but not limited to corticosteroids, IVIg, SCIg, plasma exchange, or other immunosuppressive treatments. In some embodiments, subjects treated in accordance with the methods described herein have previously been treated with IVIg. In some embodiments, subjects treated in accordance with the methods described herein have previously been treated with corticosteroids. In some embodiments, subjects treated in accordance with the methods described herein have active disease despite treatment with corticosteroids or immunoglobulins.

### Dosing

In some embodiments, the FcRn antagonist is administered at a fixed dose of about 20 mg to about 20,000 mg. In some embodiments, the FcRn antagonist is administered at a fixed dose of about 200 mg to about 20,000 mg. In some embodiments, the FcRn antagonist is administered at a fixed dose of about 300 mg to about 6000 mg. In some embodiments, the FcRn antagonist is administered at a fixed dose of about 750 mg to about 3000 mg. In some embodiments, the FcRn antagonist is administered at a fixed dose of about 1000 mg to about 2500 mg. In some embodiments, the FcRn antagonist is administered at a fixed dose of about 1000 mg to about 2000 mg. In some embodiments, the FcRn antagonist is administered at a fixed dose of about 1000 mg. In some embodiments, the FcRn antagonist is efgartigimod.

In some embodiments, the FcRn antagonist is administered at a fixed dose of 20 mg to 20,000 mg. In some embodiments, the FcRn antagonist is administered at a fixed dose of 200 mg to 20,000 mg. In some embodiments, the FcRn antagonist is administered at a fixed dose of 300 mg to 6000 mg. In some embodiments, the FcRn antagonist is administered at a fixed dose of 750 mg to 3000 mg. In some embodiments, the FcRn antagonist is administered at a fixed dose of 1000 mg to 2500 mg. In some embodiments, the FcRn antagonist is administered at a fixed dose of 1000 mg to 2000 mg. In some embodiments, the FcRn antagonist is administered at a fixed dose of 1000 mg. In some embodiments, the FcRn antagonist is efgartigimod.

In some embodiments, the FcRn antagonist is administered at a fixed dose of about 20 mg, about 50 mg, about 100 mg, about 200 mg, about 250 mg, about 300 mg, about 500 mg, about 750 mg, about 1000 mg, about 1500 mg, about 2000 mg, about 2500 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg. In some embodiments, the FcRn antagonist is efgartigimod.

In some embodiments, the FcRn antagonist is administered at a fixed dose of 20 mg, 50 mg, 100 mg, 200 mg, 250 mg, 300 mg, 500 mg, 750 mg, 1000 mg, 1500 mg, 2000 mg, 2500 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg. In some embodiments, the FcRn antagonist is efgartigimod.

In some embodiments, the FcRn antagonist is administered at a dose of about 0.2 mg/kg to about 200 mg/kg. In some embodiments, the FcRn antagonist is administered at a dose of about 2 mg/kg to about 200 mg/kg. In some embodiments, the FcRn antagonist is administered at a dose of about 2 mg/kg to about 120 mg/kg. In some embodiments, the FcRn antagonist is administered at a dose of about 3 mg/kg to about 60 mg/kg. In some embodiments, the FcRn antagonist is administered at a dose of about 10 mg/kg to about 25 mg/kg. In some embodiments, the FcRn antagonist is efgartigimod.

In some embodiments, the FcRn antagonist is administered at a dose of 0.2 mg/kg to 200 mg/kg. In some embodiments, the FcRn antagonist is administered at a dose of about 2 mg/kg to about 200 mg/kg. In some embodiments, the FcRn antagonist is administered at a dose of 2 mg/kg to 120 mg/kg. In some embodiments, the FcRn antagonist is administered at a dose of 3 mg/kg to 60 mg/kg. In some embodiments, the FcRn antagonist is administered at a dose of 10 mg/kg to 25 mg/kg. In some embodiments, the FcRn antagonist is efgartigimod.

In some embodiments, the FcRn antagonist is administered at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered subcutaneously. In some embodiments, the FcRn antagonist is administered subcutaneously once weekly. In some embodiments, the FcRn antagonist is administered subcutaneously once every two weeks. In some embodiments, the FcRn antagonist is administered subcutaneously once every three weeks, once every four weeks, once monthly, or once every six weeks. In some embodiments, the FcRn antagonist is efgartigimod.

In some embodiments, FcRn antagonist is administered subcutaneously at a fixed dose of about 20 mg to about 20,000 mg. In some embodiments, FcRn antagonist is administered subcutaneously at a fixed dose of about 100 mg to about 10,000 mg once weekly, once every two weeks, once every three weeks, once every four weeks, once monthly, or once every six weeks. In some embodiments, the FcRn antagonist is administered subcutaneously at a fixed dose of 750 mg to 3000 mg once weekly, once every two weeks, once every three weeks, once every four weeks, once monthly, or once every six weeks. In some embodiments, the FcRn antagonist is administered subcutaneously at a fixed dose of 1000 mg to 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of about 20 mg, about 50 mg, about 100 mg, about 250 mg, about 500 mg, about 750 mg, about 1000 mg, about 1500 mg, about 2000 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of 20 mg, 50 mg, 100 mg, 250 mg, 500 mg, 750 mg, 1000 mg, 1500 mg, 2000 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg once weekly, once every two weeks, once every three weeks, once every four weeks, once monthly, or once every six weeks. In some embodiments, the FcRn antagonist is administered subcutaneously at a fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously once weekly or once every two weeks at a fixed dose of about 750 mg to about 3000 mg. In some embodiments, the FcRn antagonist is administered subcutaneously once weekly or once every two weeks at a fixed dose of about 1000 mg to about 2000 mg. In some embodiments, the FcRn antagonist is administered subcutaneously at a fixed dose of about 1000 mg or about 2000 mg once weekly or once every two weeks. In some embodiments, the FcRn antagonist is efgartigimod.

In some embodiments, the FcRn antagonist is administered subcutaneously once weekly or once every two weeks at a fixed dose of 750 mg to 3000 mg. In some embodiments, the FcRn antagonist is administered subcutaneously once weekly or once every two weeks at a fixed dose of 1000 mg to 2000 mg. In some embodiments, the FcRn antagonist is administered subcutaneously at a fixed dose of 1000 mg or 2000 mg once weekly or once every two weeks. In some embodiments, the FcRn antagonist is efgartigimod.

In some embodiments, the FcRn antagonist is administered subcutaneously once weekly at a fixed dose of about 1000 mg. In some embodiments, efgartigimod is administered subcutaneously once weekly at a fixed dose of about 1000 mg. In some embodiments, the FcRn antagonist is administered subcutaneously once every two weeks at a fixed dose of about 1000 mg. In some embodiments, efgartigimod is administered subcutaneously once every two weeks at a fixed dose of about 1000 mg.

In some embodiments, the FcRn antagonist is first administered subcutaneously at a fixed dose of about 1000 mg twice on the same day. In some embodiments, the FcRn antagonist is first administered subcutaneously at a fixed dose of 1000 mg twice on the same day. In some embodiments, the FcRn antagonist is efgartigimod.

In some embodiments, the FcRn antagonist is administered subcutaneously at a fixed dose of about 2000 mg for the first two administrations and is subsequently administered subcutaneously at a fixed dose of about 1000 mg. In some embodiments, the FcRn antagonist is administered subcutaneously at a fixed dose of 2000 mg for the first two administrations and is subsequently administered subcutaneously at a fixed dose of 1000 mg. In some embodiments, the first two administrations of the FcRn antagonist are subcutaneous administrations at a fixed dose of about 1000 mg administered twice on the same day. In some embodiments, the first two administrations of the FcRn antagonist are subcutaneous administrations at a fixed dose of 1000 mg administered twice on the same day. In some embodiments, the FcRn antagonist is efgartigimod.

In some embodiments, the FcRn antagonist is administered subcutaneously once weekly at a fixed dose of about 750 mg to about 1750 mg. In some embodiments, the FcRn antagonist is administered subcutaneously once weekly at a fixed dose of about 800 mg to about 1200 mg. In some embodiments, the FcRn antagonist is administered subcutaneously once weekly at a fixed dose of about 750 mg. In some embodiments, the FcRn antagonist is administered subcutaneously once weekly at a fixed dose of about 800 mg. In some embodiments, the FcRn antagonist is administered subcutaneously once weekly at a fixed dose of about 1000 mg. In some embodiments, the FcRn antagonist is administered subcutaneously once weekly at a fixed dose of about 1200 mg. In some embodiments, the FcRn antagonist is administered subcutaneously once weekly at a fixed dose of about 1250 mg. In some embodiments, the FcRn antagonist is administered subcutaneously once weekly at a fixed dose of about 1500 mg. In some embodiments, the FcRn antagonist is administered subcutaneously once weekly at a fixed dose of about 1750 mg. In some embodiments, the FcRn antagonist is efgartigimod.

In some embodiments, the FcRn antagonist is administered subcutaneously once weekly at a fixed dose of 750 mg to 1750 mg. In some embodiments, the FcRn antagonist is administered subcutaneously once weekly at a fixed dose of 800 mg to 1200 mg. In some embodiments, the FcRn antagonist is administered subcutaneously once weekly at a fixed dose of 750 mg. In some embodiments, the FcRn antagonist is administered subcutaneously once weekly at a fixed dose of 800 mg. In some embodiments, the FcRn antagonist is administered subcutaneously once weekly at a fixed dose of 1000 mg. In some embodiments, the FcRn antagonist is administered subcutaneously once weekly at a fixed dose of 1200 mg. In some embodiments, the FcRn antagonist is administered subcutaneously once weekly at a fixed dose of 1250 mg. In some embodiments, the FcRn antagonist is administered subcutaneously once weekly at a fixed dose of 1500 mg. In some embodiments, the FcRn antagonist is administered subcutaneously once weekly at a fixed dose of 1750 mg. In some embodiments, the FcRn antagonist is efgartigimod.

In some embodiments, the FcRn antagonist is administered subcutaneously once weekly at a fixed dose of 1008 mg. In some embodiments, the FcRn antagonist is efgartigimod.

In some embodiments, the FcRn antagonist is administered subcutaneously once weekly at a dose of about 10 mg/kg to about 25 mg/kg. In some embodiments, the FcRn antagonist is administered subcutaneously once weekly at a dose of about 10 mg/kg. In some embodiments, the FcRn antagonist is administered subcutaneously once weekly at a dose of about 15 mg/kg. In some embodiments, the FcRn antagonist is administered subcutaneously once weekly at a dose of about 20 mg/kg. In some embodiments, the FcRn antagonist is administered subcutaneously once weekly at a dose of about 25 mg/kg. In some embodiments, the FcRn antagonist is efgartigimod.

In some embodiments, the FcRn antagonist is administered subcutaneously once weekly at a dose of 10 mg/kg to 25 mg/kg. In some embodiments, the FcRn antagonist is administered subcutaneously once weekly at a dose of 10 mg/kg. In some embodiments, the FcRn antagonist is administered subcutaneously once weekly at a dose of 15 mg/kg. In some embodiments, the FcRn antagonist is administered subcutaneously once weekly at a dose of 20 mg/kg. In some embodiments, the FcRn antagonist is administered subcutaneously once weekly at a dose of 25 mg/kg. In some embodiments, the FcRn antagonist is efgartigimod.

In some embodiments, the FcRn antagonist is administered intravenously. In some embodiments, the FcRn antagonist is administered intravenously once weekly, once every two weeks, once every three weeks, once every four weeks, once monthly, or once every six weeks. In some embodiments, the FcRn antagonist is efgartigimod.

In some embodiments, the FcRn antagonist is administered intravenously once weekly or once every two weeks at a dose of about 0.2 mg/kg to about 200 mg/kg. In some embodiments, the FcRn antagonist is administered intravenously once weekly or once every two weeks at a dose of about 2 mg/kg to about 200 mg/kg. In some embodiments, the FcRn antagonist is administered intravenously once weekly or once every two weeks at a dose of about 2 mg/kg to about 120 mg/kg. In some embodiments, the FcRn antagonist is administered intravenously once weekly or once every two weeks at a dose of about 3 mg/kg to about 60 mg/kg. In some embodiments, the FcRn antagonist is administered intravenously once weekly or once every two weeks at a dose of about 10 mg/kg to about 25 mg/kg. In some embodiments, the FcRn antagonist is efgartigimod.

In some embodiments, the FcRn antagonist is administered intravenously once weekly or once every two weeks at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered intravenously once weekly or once every two weeks at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously once weekly or once every two weeks at a dose of about 10 mg/kg to about 30 mg/kg. In some embodiments, the FcRn antagonist is administered intravenously once weekly or once every two weeks at a dose of about 10 mg/kg to about 25 mg/kg. In some embodiments, the FcRn antagonist is administered intravenously once weekly or once every two weeks at a dose of about 10 mg/kg. In some embodiments, the FcRn antagonist is administered intravenously once weekly or once every two weeks at a dose of about 15 mg/kg. In some embodiments, the FcRn antagonist is administered intravenously once weekly or once every two weeks at a dose of about 20 mg/kg. In some embodiments, the FcRn antagonist is administered intravenously once weekly or once every two weeks at a dose of about 25 mg/kg. In some embodiments, the FcRn antagonist is administered intravenously once weekly or once every two weeks at a dose of about 30 mg/kg. In some embodiments, the FcRn antagonist is administered intravenously once weekly or once every two weeks at a dose of 10 mg/kg to 30 mg/kg. In some embodiments, the FcRn antagonist is administered intravenously once weekly or once every two weeks at a dose of 10 mg/kg to 25 mg/kg. In some embodiments, the FcRn antagonist is administered intravenously once weekly or once every two weeks at a dose of 10 mg/kg. In some embodiments, the FcRn antagonist is administered intravenously once weekly or once every two weeks at a dose of 15 mg/kg. In some embodiments, the FcRn antagonist is administered intravenously once weekly or once every two weeks at a dose of 20 mg/kg. In some embodiments, the FcRn antagonist is administered intravenously once weekly or once every two weeks at a dose of 25 mg/kg. In some embodiments, the FcRn antagonist is administered intravenously once weekly or once every two weeks at a dose of 30 mg/kg. In some embodiments, the FcRn antagonist is efgartigimod.

In some embodiments, the FcRn antagonist is first administered intravenously and is subsequently administered subcutaneously. In some embodiments, the FcRn antagonist is first administered intravenously and is subsequently administered subcutaneously at fixed dose of 100 mg to 10,000 mg once weekly, once every two weeks, once every three weeks, once every four weeks, once monthly, or once every six weeks. In some embodiments, the FcRn antagonist is first administered intravenously and is subsequently administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, one or more doses of the FcRn antagonist are administered intravenously and subsequent doses of the FcRn antagonist are administered subcutaneously. In some embodiments, one or more doses of the FcRn antagonist are administered intravenously and subsequent doses of the FcRn antagonist are administered subcutaneously at fixed dose of 100 mg to 10,000 mg once weekly, once every two weeks, once every three weeks, once every four weeks, once monthly, or once every six weeks. In some embodiments, one or more doses of the FcRn antagonist are administered intravenously and subsequent doses of the FcRn antagonist are administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is first administered either by or under the supervision of a healthcare professional for at least 5 administrations. In some embodiments, subsequent administrations of a clinically proven safe and clinically proven effective amount of efgartigimod PH20 are given by a healthcare professional or by a patient or caregiver.

In some embodiments, the FcRn antagonist is administered for 61 weeks or less, 52 weeks or less or 48 weeks or less. In some embodiments, the FcRn antagonist is administered for at least 4 weeks. In some embodiments, the FcRn antagonist is administered for at least 12 weeks. In some embodiments, the FcRn antagonist is administered for at least 48 weeks. In some embodiments, the FcRn antagonist is administered for at least 52 weeks. In some embodiments, the FcRn antagonist is administered for at least 61 weeks.

In some embodiments, the FcRn antagonist is administered once weekly until disease control or complete remission is achieved.

In some embodiments, the FcRn antagonist is administered in an induction phase followed by a maintenance phase. In certain embodiments, during the induction phase, the FcRn antagonist is administered once weekly and during the maintenance phase, the FcRn antagonist dosing interval is lengthened, e.g., to once every two weeks, once every three weeks or once every four weeks. For embodiments wherein the induction phase involves administration of an FcRn antagonist at a dose or at a dosing frequency that is higher than during the maintenance phase, any of the specific dosing regimens described herein may be applied during induction and maintenance phases.

For example, in certain embodiments, during the induction phase, the FcRn antagonist is administered subcutaneously once weekly and during the maintenance phase, the FcRn antagonist is administered subcutaneously once every two weeks. In some embodiments, during the induction phase, the FcRn antagonist is administered subcutaneously once weekly at a fixed dose of about 1000 mg and during the maintenance phase, the FcRn antagonist is administered subcutaneously once every two weeks at a fixed dose of about 1000 mg. In some embodiments, the FcRn antagonist is efgartigimod. In some embodiments, the FcRn antagonist is self-administered during the maintenance phase.

The induction phase may be at least 4 weeks, at least 8 weeks or at least 12 weeks. The induction phase may be 61 weeks or less, 52 weeks or less, 48 weeks or less. The induction phase may be between 4 weeks and 61 weeks, between 8 weeks and 52 weeks or between 12 weeks and 48 weeks. In some embodiments, the induction phase ends based on clinical evaluation by, for example, a healthcare provider. In some embodiments, the induction phase ends when the subject exhibits ECI.

In some embodiments, the FcRn antagonist is first administered subcutaneously at a fixed dose of about 1000 mg once weekly and is subsequently administered subcutaneously at a fixed dose of about 1000 mg once every two weeks based on clinical evaluation. In some embodiments, the clinical evaluation is performed by a healthcare provider. In some embodiments, the FcRn antagonist is first administered subcutaneously at a fixed dose of about 1000 mg once weekly and is subsequently administered subcutaneously at a fixed dose of about 1000 mg once every two weeks when ECI is demonstrated in the subject. In some embodiments, once weekly subcutaneous administrations at a fixed dose of about 1000 mg are resumed upon worsening of symptoms.

In some embodiments, the FcRn antagonist is first administered subcutaneously at a fixed dose of 1000 mg once weekly and is subsequently administered subcutaneously at a fixed dose of 1000 mg once every two weeks based on clinical evaluation. In some embodiments, the clinical evaluation is performed by a healthcare provider. In some embodiments, the FcRn antagonist is first administered subcutaneously at a fixed dose of 1000 mg once weekly and is subsequently administered subcutaneously at a fixed dose of 1000 mg once every two weeks when ECI is demonstrated in the subject. In some embodiments, once weekly subcutaneous administrations at a fixed dose of 1000 mg are resumed upon worsening of symptoms.

In some embodiments, the FcRn antagonist is first administered subcutaneously at a fixed dose of 1008 mg once weekly and is subsequently administered subcutaneously at a fixed dose of 1008 mg once every two weeks based on clinical evaluation. In some embodiments, the clinical evaluation is performed by a healthcare provider. In some embodiments, the FcRn antagonist is first administered subcutaneously at a fixed dose of 1008 mg once weekly and is subsequently administered subcutaneously at a fixed dose of 1008 mg once every two weeks when ECI is demonstrated in the subject. In some embodiments, once weekly subcutaneous administrations at a fixed dose of 1008 mg are resumed upon worsening of symptoms.

In some embodiments, treatment according to any of the dosing regimens described herein is followed by a period wherein the subject does not receive any further FcRn antagonist. Subjects receiving an FcRn antagonist according to any of the dosing regimens described herein may achieve control of disease activity (CDA), partial remission (PR), or complete remission (CR) such that treatment can be withdrawn. The subject may remain untreated during a period wherein the CDA, PR or CR has been achieved, optionally with a period of further treatment if clinical deterioration occurs. If, further to treatment according to any of the dosing regimens described herein, CDA, PR, or CR is not achieved or relapse occurs, the subject may undergo a further period of treatment with the FcRn antagonist in accordance with any of the dosing regimens described herein.

In some embodiments, the FcRn antagonist is administered subcutaneously once weekly for at least 4 weeks. In some embodiments, the FcRn antagonist is administered subcutaneously once weekly until the subject demonstrates EC. In some embodiments, the FcRn antagonist is administered once weekly until the subject demonstrates ECI during two consecutive measurements, optionally two consecutive measurements taken about 1 week apart. In some embodiments, the FcRn antagonist is administered subcutaneously once weekly until the subject demonstrates ECI twice in two weeks. In some embodiments, ECI is clinical improvement in one or more of I-RODS, Mean Grip Strength, INCAT, or aINCAT score. In some embodiments, ECI is a clinical improvement in one or more of I-RODS, Mean Grip Strength, or INCAT score.

In some embodiments, the FcRn antagonist is administered subcutaneously once weekly until the subject demonstrates ECMD. In some embodiments, the ECMD is one or more of an increase in aINCAT score of ≥1 points, a decrease in I-RODS of ≥4 points (using the centile metric), or a decrease in mean grip strength of ≥8 kPa in one hand using a handheld vigorimeter. In some embodiments, the ECMD is an increase in aINCAT score of ≥1 points. In some embodiments, the ECMD is an increase in aINCAT score of ≥1 points during two consecutive measurements, optionally two consecutive measurements taken about 1 week apart. In some embodiments, the ECMD is an increase in aINCAT score of ≥1 points twice in two weeks. In some embodiments, the ECMD is an increase in aINCAT score of ≥2 points.

In some embodiments, a clinically proven safe and clinically proven effective amount of efgartigimod PH20 is administered subcutaneously. In some embodiments, a clinically proven safe and clinically proven effective amount of efgartigimod PH20 is administered subcutaneously once weekly or once every other week. In some embodiments, a clinically proven safe and clinically proven effective amount of efgartigimod PH20 is administered subcutaneously once weekly. In some embodiments, a clinically proven safe and clinically proven effective amount of efgartigimod PH20 is administered subcutaneously once weekly for at least 4 weeks. In some embodiments, a clinically proven safe and clinically proven effective amount of efgartigimod PH20 is administered subcutaneously once weekly until the subject demonstrates EC. In some embodiments, a clinically proven safe and clinically proven effective amount of efgartigimod PH20 is administered once weekly until the subject demonstrates ECI during two consecutive measurements, optionally two consecutive measurements taken about 1 week apart. In some embodiments, a clinically proven safe and clinically proven effective amount of efgartigimod PH20 is administered subcutaneously once weekly until the subject demonstrates ECI twice in two weeks. In some embodiments, ECI is clinical improvement in one or more of I-RODS, Mean Grip Strength, INCAT, or aINCAT score. In some embodiments, ECI is a clinical improvement in one or more of I-RODS, Mean Grip Strength, or INCAT score.

In some embodiments, a clinically proven safe and clinically proven effective amount of efgartigimod PH20 is administered subcutaneously once weekly until the subject demonstrates ECMD. In some embodiments, the ECMD is one or more of an increase in aINCAT score of ≥1 points, a decrease in I-RODS of ≥4 points (using the centile metric), or a decrease in mean grip strength of ≥8 kPa in one hand using a handheld vigorimeter. In some embodiments, the ECMD is an increase in aINCAT score of ≥1 points. In some embodiments, the ECMD is an increase in aINCAT score of ≥1 points during two consecutive measurements, optionally two consecutive measurements taken about 1 week apart. In some embodiments, the ECMD is an increase in aINCAT score of ≥1 points twice in two weeks. In some embodiments, the ECMD is an increase in aINCAT score of ≥2 points.

In some embodiments, a clinically proven safe and clinically proven effective amount of efgartigimod PH20 is first administered subcutaneously once weekly and is subsequently administered subcutaneously once every two weeks based on clinical evaluation. In some embodiments, the clinical evaluation is performed by a healthcare provider. In some embodiments, a clinically proven safe and clinically proven effective amount of efgartigimod PH20 is first administered subcutaneously once weekly and is subsequently administered subcutaneously once every two weeks when ECI is demonstrated in the subject. In some embodiments, once weekly subcutaneous administrations of a clinically proven safe and clinically proven effective amount of efgartigimod PH20 are resumed upon worsening of symptoms. In some embodiments, a clinically proven safe and clinically proven effective amount of efgartigimod PH20 is administered subcutaneously over approximately 30 to 90 seconds. In some embodiments, a clinically proven safe and clinically proven effective amount of efgartigimod PH20 is first administered either by or under the supervision of a healthcare professional for at least 5 administrations. In some embodiments, subsequent administrations of a clinically proven safe and clinically proven effective amount of efgartigimod PH20 are given by a healthcare professional or by a patient or caregiver.

In some embodiments, a clinically proven safe and clinically proven effective amount of efgartigimod PH20 is 1008 mg/11,200 units. In some embodiments, a clinically proven safe and clinically proven effective amount of efgartigimod PH20 is 1000 mg/11,200 units.

In some embodiments, the FcRn antagonist is rozanolixizumab. In some embodiments, rozanolixizumab is administered subcutaneously or intravenously. In some embodiments, rozanolixizumab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, rozanolixizumab is administered once weekly, once every two weeks, once every three weeks, once every four weeks or once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is nipocalimab. In some embodiments, nipocalimab is administered subcutaneously or intravenously. In some embodiments, nipocalimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, nipocalimab is administered once weekly, once every two weeks, once every three weeks, once every four weeks or once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is orilanolimab. In some embodiments, orilanolimab is administered subcutaneously or intravenously. In some embodiments, orilanolimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, orilanolimab is administered once weekly, once every two weeks, once every three weeks, once every four weeks, once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered intravenously at a dose of about 30 mg/kg once weekly for three weeks and then at a dose of 10 mg/kg administered intravenously every other week.

In some embodiments, the FcRn antagonist is batoclimab or IMVT-1402. In some embodiments, batoclimab or IMVT-1402 is administered subcutaneously or intravenously. In some embodiments, batoclimab or IMVT-1402 is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, batoclimab or IMVT-1402 is administered once weekly, once every two weeks, once every three weeks, once every four weeks or once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

### Treatment Outcomes

Treatment of CIDP will typically be accompanied by a change, specifically an improvement, in disease status. In some embodiments, administration of an FcRn antagonist to a subject with CIDP will lead to control of disease activity (CDA), partial remission (PR), or complete remission (CR), as defined herein. In some embodiments, complete remission is achieved. In some embodiments CDA, PR or CR is sustained in the subject for at least two months, at least three months, at least four months, at least five months or at least six months.

In some embodiments, CDA, PR, or CR is achieved within 12 weeks or less of first receiving an FcRn antagonist as described herein. In some embodiments, CDA, PR, or CR is achieved within 8 weeks or less of first receiving an FcRn antagonist as described herein. In some embodiments, CDA, PR, or CR is achieved within 6 weeks or less of first receiving an FcRn antagonist as described herein. In some embodiments, CDA, PR or CR is achieved within 4 weeks or less of first receiving an FcRn antagonist as described herein. In some embodiments, CDA, PR, or CR is achieved within 3 weeks or less of first receiving an FcRn antagonist as described herein. In some embodiments, the FcRn antagonist is efgartigimod.

In some embodiments, once CDA, PR, or CR is achieved, continuous administration of the FcRn antagonist prevents deterioration of symptoms. This prevention may be achieved for the duration of the treatment i.e., all the time that the subject is receiving the FcRn antagonist. The absence of a deterioration in symptoms may be measured using any of the assessment methods described herein including but not limited to: (i) the INCAT Disability Scale (Merkies et al., 2002); (ii) the MRC Scale (Vanhoutte et al., 2012); (iii) the I-RODS (van Nes et al., 2011); (iv) the Mean Grip Strength test (Vanhoutte et al., 2013); and (v) the TUG test.

In certain embodiments, treatment with an FcRn antagonist achieves CDA, PR and/or CR and/or prevents or delays relapse i.e., a clinical deterioration of symptoms. In certain embodiments, disease relapse is prevented or delayed by at least 4 weeks, at least 6 weeks, at least 8 weeks, at least 10 weeks, at least 12 weeks, at least 16 weeks, at least 20 weeks.

In some embodiments, the treatment prevents or delays CIDP relapse after achieving PR or CR from CIDP for at least 4, 6, 8, 10, 12, 14, 16, 18, 20, 22, 24, 26, 28, 30, 32, 34, 36, 38, 40, 42, 44, 46, 48, 50, or 52 weeks.

In some embodiments, the treatment prevents or delays CIDP relapse for the duration of the treatment i.e., there is no deterioration in symptoms for the period that the subject is receiving the FcRn antagonist.

In some embodiments, the treatment prevents or delays relapse following cessation of the treatment i.e., for a time period after the subject has stopped receiving the FcRn antagonist. In some embodiments, the treatment prevents or delays relapse following cessation of the treatment for at least 4 weeks, at least 6 weeks, at least 8 weeks, at least 10 weeks, at least 12 weeks, at least 16 weeks, at least 20 weeks. In some embodiments, the treatment prevents or delays relapse following cessation of the treatment for at least 20 weeks.

In some embodiments, treatment-naïve subjects are treated in accordance with the methods described herein and partial remission (PR) or complete remission is achieved. For these embodiments, subjects may experience a prevention or delay in relapse for the duration of the treatment (when the FcRn antagonist treatment continues after PR or CR is achieved). For these embodiments, treatment-naïve subjects may alternatively or in addition, experience a prevention or delay in relapse following cessation of the treatment.

In some embodiments, the treatment reduces the risk of relapse by at least 60% as compared with subjects not treated with an FcRn antagonist. In some embodiments, the treatment reduces the risk of relapse following cessation of the treatment by at least 60% as compared with subjects not treated with an FcRn antagonist. In some embodiments, the treatment reduces the risk of relapse by about 61% as compared with subjects not treated with an FcRn antagonist. In some embodiments, the treatment reduces the risk of relapse following cessation of the treatment by about 61% as compared with subjects not treated with an FcRn antagonist. In some embodiments, the treatment reduces the risk of relapse by 61% as compared with subjects not treated with an FcRn antagonist. In some embodiments, the treatment reduces the risk of relapse following cessation of the treatment by 61% as compared with subjects not treated with an FcRn antagonist.

In some embodiments, subjects treated in accordance with the methods described herein show an improvement in one or more CIDP symptoms following administration of the FcRn antagonist. Improvements in symptoms may be assessed using any of the using any of the assessment methods described herein including but not limited to: (i) the INCAT Disability Scale (Merkies et al., 2002); (ii) the MRC Scale (Vanhoutte et al., 2012); (iii) the I-RODS (van Nes et al., 2011); (iv) the Mean Grip Strength test (Vanhoutte et al., 2013); and (v) the TUG test. In some embodiments, an improvement in one or more CIDP symptoms is achieved within 12 weeks or less of first receiving an FcRn antagonist as described herein. In some embodiments, an improvement in one or more CIDP symptoms is achieved within 8 weeks or less of first receiving an FcRn antagonist as described herein. In some embodiments, an improvement in one or more CIDP symptoms is achieved within 6 weeks or less of first receiving an FcRn antagonist as described herein. In some embodiments, an improvement in one or more CIDP symptoms is achieved within 4 weeks or less of first receiving an FcRn antagonist as described herein. In some embodiments, an improvement in one or more CIDP symptoms is achieved within 3 weeks or less of first receiving an FcRn antagonist as described herein. In some embodiments, an improvement in one or more CIDP symptoms is achieved within 9 days or less of first receiving an FcRn antagonist as described herein. In some embodiments, an improvement in at least one of aINCAT score, I-RODS, or Grip Strength is achieved within 9 days from first receiving an FcRn antagonist as described herein.

In some embodiments, subjects treated in accordance with the methods described herein show an improvement in symptoms, as compared with baseline, i.e., before treatment, as measured using the INCAT Disability Scale. In some embodiments, the subject shows a decrease (i.e., an improvement) in INCAT score of 1 or more. In some embodiments, the subject shows a decrease (i.e., an improvement) in aINCAT score of 1 or more. In some embodiments, the subject shows a decrease (i.e., an improvement) in aINCAT score of 2 or more. In some embodiments, the subject shows a decrease (i.e., an improvement) in aINCAT score of 3 or more. In some embodiments, the subject shows a decrease (i.e., an improvement) in aINCAT score of 4 or more. In some embodiments, the subject shows a decrease (i.e., an improvement) in aINCAT score of 5 or more. In some embodiments, the subject shows a decrease (i.e., an improvement) in aINCAT score of 6 or more. In some embodiments, the subject shows a decrease (i.e., an improvement) in aINCAT score of 7 or more. In some embodiments, the subject shows a decrease (i.e., an improvement) in aINCAT score of 8 or more. In some embodiments, the subject shows a decrease (i.e., an improvement) in aINCAT score of 9 or more.

In some embodiments, the subject shows a decrease in aINCAT score such that the aINCAT score following FcRn antagonist administration is 5 or less, 4 or less, 3 or less, 2 or less, 1 or less. The subject may show improvements in upper limb performance, lower limb performance or both, as measured using the aINCAT score.

In some embodiments, subjects treated in accordance with the methods described herein show an improvement in symptoms as measured using the MRC Scale. As described elsewhere herein, the MRC scale is a 5-point scale (0-5) wherein 0 indicates complete paralysis and 5 indicates normal strength. This scale is applied bilaterally to 6 muscle groups of the upper and lower limbs (1. Arm abductors, 2. Elbow flexors, 3. Wrist extensors, 4. Hip flexors, 5. Knee extensors and 6. Foot dorsiflexors) in order to obtain a summed score ranging from 0 to 60. In some embodiments, the subject shows an increase (i.e., an improvement) in MRC score of 2 or more, 3 or more, 5 or more, 10 or more, 20 or more, 30 or more, 40 or more. The subject may show improvements in upper limb performance, lower limb performance or both, as measured using the MRC scale.

In some embodiments, subjects treated in accordance with the methods described herein show an improvement in symptoms as measured using the I-RODS. As described elsewhere herein, I-RODS is a 24-item scale, with each item representing a common daily activity that range from very difficult to do, like running or dancing, to very easy to do, like eating or reading a book. Higher scores indicate less disability. The raw scores of the I-RODS (range: 0 to 48) are typically converted to a centile metric score, ranging from 0 (most severe activity and social participation restrictions) to 100 (no activity and social participation limitations). In some embodiments, the subject shows an increase (i.e., an improvement) in I-RODS centile metric score of 4 or more, 8 or more, 12 or more, 20 or more.

In some embodiments, subjects treated in accordance with the methods described herein show an improvement in symptoms as measured using the Mean Grip Strength test. Mean grip strength can be measured using a handheld Martin vigorimeter. In some embodiments, the subject shows an increase in mean grip strength of ≥8 kPa, ≥16 kPa, ≥24 kPa following administration of the FcRn antagonist. Mean grip strength is typically measured by testing the subject's dominant hand.

In some embodiments, subjects treated in accordance with the methods described herein show an improvement in symptoms as measured using the TUG test. The TUG test is a simple test used to assess a person's mobility and requires both static and dynamic balance. In the TUG test, the time expended to rise from a chair, walk 3 meters, turn around, walk back to the chair, and sit down is measured. In some embodiments, the subject shows an improvement in the TUG test by a decrease in the time taken to complete the test of 5% or more, 10% or more, 15% or more, 20% or more, 25% or more, 30% or more.

In some embodiments, subjects show an improvement in quality of life (QoL), as measured in accordance with any of the QoL scales/tests described herein. In some embodiments, QoL is assessed by one or more of the following: EuroQoL 5-Dimension 5-Level (EQ-5D-5L); Brief Pain Inventory Short Form (BPI-SF); (9-item) Treatment Satisfaction Questionnaire for Medication (TSQM-9); Rasch-transformed-Fatigue Severity Scale (RT-FSS); Hospital Anxiety and Depression Scale (HADS); and Patient Global Impression of Change (PGIC).

In some embodiments, subjects show reductions in serum levels of one or more of: (i) total IgG levels; (ii) autoantibody levels; (iii) cytokine levels; (iv) chemokine levels; (v) immune complexes, following administration of the FcRn antagonist. Reductions may be measured using any suitable techniques known to those skilled in the art.

In some embodiments, subjects show a reduction in serum levels of one or more autoantibodies following administration of the FcRn antagonist. Such autoantibodies may be selected from the group consisting of: anti-GM1 antibodies; anti-LM-1 antibodies; Anti-NF-155 antibodies; anti-CNTN1 antibodies; anti-Caspr-1 antibodies; and anti-myelinated nerve antibodies.

FcRn also binds to and recycles serum albumin, a modulator of serum cholesterol levels. Certain FcRn antagonists, specifically certain anti-FcRn antibodies, have been found to reduce serum albumin levels and consequently increase serum cholesterol levels in human subjects, both of which are undesirable.

In some embodiments, subjects treated in accordance with the methods described herein do not experience a decrease in the level of serum albumin following administration of the FcRn antagonist. In some embodiments, a decrease in the level of serum albumin of less than about 1%, 2%, 3%, 4%, or 5% compared to baseline albumin level is observed. In an embodiment, a decrease in the level of serum albumin of less than about 10% compared to baseline albumin level is observed. In some embodiments, subjects treated in accordance with the methods described herein do not experience an increase in serum cholesterol levels following administration of the FcRn antagonist. In some embodiments, an increase in the level of serum cholesterol of less than about 1%, 2%, 3%, 4%, or 5% compared to baseline cholesterol level is observed. In an embodiment, an increase in the level of serum cholesterol of less than about 10% compared to baseline serum cholesterol level is observed. Serum cholesterol may be measured in the form of total cholesterol, high-density lipoprotein (HDL) cholesterol and/or the ratio of total cholesterol:HDL. Advantageously, efgartigimod does not decrease serum albumin levels. Thus, in some embodiments, the FcRn antagonist is efgartigimod.

In some embodiments, subjects treated in accordance with the methods described herein do not experience significant proteinuria i.e., significant or abnormal levels of protein in the urine. Proteinuria may be quantified by albumin/creatinine ratio (ACR) or protein/creatinine ratio (PCR). In some embodiments, subjects treated in accordance with the methods described herein have an ACR of 15 mg/mmol or less, preferably 3 mg/mmol or less.

In some embodiments, subjects treated in accordance with the methods described herein, particularly subjects receiving the FcRn antagonist via a subcutaneous route of administration, experience minimal injection site reactions. Injection site reactions (ISRs) are a well-characterised phenomenon in the administration of medical agents (e.g., biological agents) and include swelling, erythema, pruritus, and pain around the site of injection.

In some embodiments, treatment in accordance with the methods described herein exhibits a safety profile that obviates the need for ongoing monitoring of standard patient parameters, particularly patient parameters routinely monitored during treatment of CIDP patients. In some embodiments, treatment in accordance with the methods described herein exhibits a safety profile that obviates the need for ongoing or regular monitoring of serum albumin levels, serum cholesterol levels and/or proteinuria. In some embodiments, treatment in accordance with the methods described herein exhibits a safety profile that obviates the need for patient pre-medication.

In some embodiments, a method for treating CIDP in subjects in a patient population is provided. In some embodiments, the method comprises administering an FcRn antagonist as described herein to the subjects in the patient population. In some embodiments, the method comprises administering an FcRn antagonist as described herein co-formulated with hyaluronidase to the subjects in the patient population. In some embodiments, the method comprises administering efgartigimod PH20, or a biosimilar version thereof, to the patient population. In some embodiments, the method comprises administering 1008 mg/11,200 units of efgartigimod PH20, or a biosimilar version thereof, to the patient population. In some embodiments, the method comprises administering the FcRn antagonist (e.g., efgartigimod) subcutaneously once weekly.

In some embodiments, the patient population demonstrates ECI in 66.5% in subjects in the patient population. In some embodiments, the patient population demonstrates ECI in 66.5% in subjects in the patient population following administration of the FcRn antagonist. In some embodiments, the patient population demonstrates ECI in 66.5% of the subjects in the patient population. In some embodiments, the patient population demonstrates ECI in 66.5% of the subjects in the patient population following administration of the FcRn antagonist. In some embodiments, the patient population achieves ECI within 31-51 days of first receiving the FcRn antagonist. In some embodiments, the patient population achieves ECI within 43 days. In some embodiments, the patient population achieves ECI within 43 days of first receiving the FcRn antagonist. In some embodiments, the patient population comprises 322 subjects. In some embodiments, the FcRn antagonist is efgartigimod PH20, or a biosimilar version thereof.

In some embodiments, the patient population demonstrates evidence of improvement during two consecutive measurements in 69% of the subjects in the patient population following administration of the FcRn antagonist. In some embodiments, the evidence of improvement is selected from aINCAT improvement ≥1 point, I-RODS improvement ≥4 points, or mean grip strength improvement ≥8 kPa. In some embodiments, the patient population comprises 322 subjects. In some embodiments, the FcRn antagonist is efgartigimod PH20, or a biosimilar version thereof.

In some embodiments, anti-efgartigimod alfa antibodies are detected in 6% of the subjects in the patient population following administration of the FcRn antagonist. In some embodiments, anti-efgartigimod alfa antibodies are detected in 6% of the subjects in the patient population following administration of the FcRn antagonist for up to 12 weeks. In some embodiments, neutralizing anti-efgartigimod alfa antibodies are detected in 0.3% of the subjects in the patient population following administration of the FcRn antagonist. In some embodiments, neutralizing anti-efgartigimod alfa antibodies are detected in 0.3% of the subjects in the patient population following administration of the FcRn antagonist for up to 12 weeks. In some embodiments, anti-efgartigimod alfa antibodies are detected in 2% of the subjects in the patient population following administration of the FcRn antagonist. In some embodiments, anti-efgartigimod alfa antibodies are detected in 2% of the subjects in the patient population following administration of the FcRn antagonist for up to 48 weeks. In some embodiments, the FcRn antagonist is efgartigimod PH20, or a biosimilar version thereof.

In some embodiments, the subjects in the patient population remain relapse-free longer (e.g., significantly longer) than subjects who did not receive efgartigimod PH20, or a biosimilar version thereof. In some embodiments, the subjects in the patient population remain relapse-free of CIDP symptoms longer (e.g., significantly longer) than subjects who did not receive efgartigimod PH20, or a biosimilar version thereof.

In some embodiments, the subjects in the patient population experience a longer time to clinical deterioration compared to subjects who did not receive efgartigimod PH20, or a biosimilar version thereof. In some embodiments, the clinical deterioration is an increase of ≥1 point in aINCAT score. In some embodiments, the clinical deterioration is an increase of ≥1 point in aINCAT score during two consecutive measurements. In some embodiments, the clinical deterioration is an increase of ≥2 points in aINCAT score. In some embodiments, the longer time to clinical deterioration is demonstrated by a hazard ratio of 0.394. In some embodiments, the longer time to clinical deterioration is statistically significant.

In some embodiments, the subjects in the patient population treated with efgartigimod PH20, or a biosimilar version thereof, demonstrate a 61% risk reduction for deterioration. In some embodiments, the subjects in the patient population treated with efgartigimod PH20, or a biosimilar version thereof, demonstrate a 61% risk reduction for deterioration in patients with CIDP. In some embodiments, the subjects in the patient population treated with efgartigimod PH20, or a biosimilar version thereof, demonstrate a 61% risk reduction for deterioration as compared to subjects not treated with efgartigimod PH20 (e.g., subjects treated with placebo).

In some embodiments, the mean percentage reduction from baseline in total IgG levels (e.g., serum IgG levels) in the patient population ranged between about 66.8% and about 71.6% following administration of efgartigimod PH20, or a biosimilar version thereof. In some embodiments, the mean percentage reduction from baseline in total IgG levels (e.g., serum IgG levels) in the patient population ranged between 66.8% and 71.6% following administration of efgartigimod PH20, or a biosimilar version thereof. In some embodiments, the mean percentage reduction from baseline in total IgG levels (e.g., serum IgG levels) was sustained from week 4 throughout the treatment period.

In some embodiments, a subject shows a reduction in serum level of total IgG of between about 66.8% and about 71.6% following administration of efgartigimod PH20, or biosimilar version thereof. In some embodiments, a subject shows a reduction in serum level of total IgG of between about 66.8% and about 71.6% following 4 weekly administrations of efgartigimod PH20, or biosimilar version thereof. In some embodiments, a subject shows a reduction in serum level of total IgG of between 66.8% and 71.6% following administration of efgartigimod PH20, or biosimilar version thereof. In some embodiments, a subject shows a reduction in serum level of total IgG of between 66.8% and 71.6% following 4 weekly administrations of efgartigimod PH20, or biosimilar version thereof. In some embodiments, the reduction in serum level of total IgG is sustained until once weekly administration of efgartigimod PH20, or biosimilar version thereof, is discontinued.

In some embodiments, prior to treatment with efgartigimod PH20, the patient population comprises treatment naïve CIDP patients, CIDP patients receiving immunoglobulin, and CIDP patients receiving corticosteroids.

In some embodiments, the patient population comprises patients with CIDP that is unstable and refractory to treatment (CIDP Disease Activity Score (CDAS) 5B and 5C) prior to treatment with efgartigimod PH20, and patients with CIDP that is stable on existing treatment (CDAS 3 and 4) prior to treatment with efgartigimod PH20. In some embodiments, the patient population comprises CIDP patients having disability that is mild to severe prior to treatment with efgartigimod PH20. In some embodiments, the patient population comprises CIDP patients having an INCAT score of 2-9 prior to treatment with efgartigimod PH20. In some embodiments, the patient population comprises CIDP patients having an IRODS of 11-61 prior to treatment with efgartigimod PH20. In some embodiments, the patient population comprises CIDP patients having a mean grip strength of 1-120 kPa prior to treatment with efgartigimod PH20. In some embodiments, the patient population comprises CIDP patients having a mean INCAT score of 5 prior to treatment with efgartigimod PH20. In some embodiments, the patient population comprises CIDP patients having a mean IRODS of 37 prior to treatment with efgartigimod PH20. In some embodiments, the patient population comprises CIDP patients having a mean grip strength of 39 kPa prior to treatment with efgartigimod PH20.

### Additional Therapeutic Agents

The methods described herein may include an additional step of administering to the subject an effective amount of one or more additional therapeutic agents.

In some embodiments, the methods further comprise the administration of an effective amount of one or more immunosuppressive agents. Examples include but are not limited to methotrexate, mycophenolate mofetil (MMF), azathioprine, cyclophosphamide, doxycycline, and dapsone.

In some embodiments, the methods further comprise the administration of an effective amount of one or more corticosteroids to the subject. In certain embodiments, the corticosteroid is an oral corticosteroid. Examples of oral corticosteroids include, but are not limited to, betamethasone, cortisone, dexamethasone, hydrocortisone, methylprednisolone, prednisone, prednisolone, and triamcinolone. In some embodiments, the oral corticosteroid is dexamethasone. In some embodiments, the oral corticosteroid is methylprednisolone, prednisone or prednisolone.

In an embodiment, the effective amount of the corticosteroid is administered at a dose of 2.5 mg/day to 20 mg/day. In an embodiment, the effective amount of the corticosteroid is administered at a dose of 5 mg/day to 20 mg/day. In an embodiment, the effective amount of the corticosteroid is administered at a dose of 7.5 mg/day to 20 mg/day. In an embodiment, the effective amount of the corticosteroid is administered at a dose of 8 mg/day to 15 mg/day. In an embodiment, the effective amount of the corticosteroid is administered at a dose of 10 mg/day to 15 mg/day. In an embodiment, the effective amount of the corticosteroid is administered at a dose of about 20 mg/day. In an embodiment, the effective amount of the corticosteroid is administered at a dose of about 10 mg/day. In an embodiment, the effective amount of the corticosteroid is administered at a dose of about 7.5 mg/day. In an embodiment, the effective amount of the corticosteroid is administered at a dose of about 5 mg/day. In an embodiment, the effective amount of the corticosteroid is administered at a dose of about 2.5 mg/day. In an embodiment, the effective amount of the corticosteroid is administered at a dose of about 1 mg/kg/day. In an embodiment, the effective amount of the corticosteroid is administered at a dose of about 0.75 mg/kg/day. In an embodiment, the effective amount of the corticosteroid is administered at a dose of about 0.5 mg/kg/day. In an embodiment, the effective amount of the corticosteroid is administered at a dose of about 0.4 mg/kg/day. In an embodiment, the effective amount of the corticosteroid is administered at a dose of about 0.3 mg/kg/day. In an embodiment, the effective amount of the corticosteroid is administered at a dose of about 0.25 mg/kg/day. In an embodiment, the effective amount of the corticosteroid is administered at a dose of about 0.2 mg/kg/day. In an embodiment, the effective amount of the corticosteroid is administered at a dose of about 0.15 mg/kg/day. In an embodiment, the effective amount of the corticosteroid is administered at a dose of about 0.1 mg/kg/day.

Subjects treated with corticosteroid may have their corticosteroid dosing regimen tapered prior to, during or after the treatment with the FcRn antagonist. Tapering the corticosteroid dosing regimen may refer to lowering the dose or lowering the dosing frequency of the corticosteroid.

In an embodiment, the dose of corticosteroid is reduced after CDA or remission (partial or complete) is achieved. In some embodiments, the dose of corticosteroid is reduced after CDA has been sustained for at least two weeks.

In some embodiments, the corticosteroid dosing regimen is reduced by following a tapering schedule. Examples of tapering schedules that may be used to reduce corticosteroid dosing are provided herein. One of skill in the art will appreciate that tapering schedules may vary and may be adapted depending upon multiple subject-specific factors, such as the initial corticosteroid dose, changes in CIDP symptoms, overall health of the subject, etc.

In some embodiments, the daily dose of oral corticosteroid (OCS) e.g., prednisone, is decreased from 0.5 mg/kg/day to 0.3 mg/kg/day, from 0.3 mg/kg/day to 0.2 mg/kg/day, from 0.2 mg/kg/day to 0.15 mg/kg/day and from 0.15 mg/kg/day to 0.1 mg/kg/day. In some embodiments, each daily dose of OCS e.g., prednisone is maintained for at least two weeks before the dose is further decreased. In some embodiments, the daily dose of OCS e.g., prednisone is maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 2 weeks. In some embodiments, the daily dose of OCS e.g., prednisone is maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 8 weeks. In some embodiments, each daily dose is maintained for two weeks, optionally provided that CIDP symptoms do not worsen.

In some embodiments, the daily dose of OCS e.g., prednisone is decreased from 0.75 mg/kg/day to 0.5 mg/kg/day, from 0.5 mg/kg/day to 0.3 mg/kg/day, from 0.3 mg/kg/day to 0.2 mg/kg/day, from 0.2 mg/kg/day to 0.15 mg/kg/day, and from 0.15 mg/kg/day to 0.1 mg/kg/day. In some embodiments, each daily dose of OCS e.g., prednisone is maintained for at least two weeks before the dose is further decreased. In some embodiments, the daily dose of OCS e.g., prednisone is maintained at 0.5 mg/kg/day for at least 2 weeks, then maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 2 weeks. In some embodiments, the daily dose of OCS e.g., prednisone is maintained at 0.5 mg/kg/day for at least 2 weeks, then maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 8 weeks. In some embodiments, each daily dose is maintained for two weeks, optionally provided that CIDP symptoms do not worsen.

In some embodiments, the daily dose of OCS e.g., prednisone is decreased from 1 mg/kg/day to 0.75 mg/kg/day, from 0.75 mg/kg/day to 0.5 mg/kg/day, from 0.5 mg/kg/day to 0.3 mg/kg/day, from 0.3 mg/kg/day to 0.2 mg/kg/day, from 0.2 mg/kg/day to 0.15 mg/kg/day, and from 0.15 mg/kg/day to 0.1 mg/kg/day. In some embodiments, each daily dose of OCS e.g., prednisone is maintained for at least two weeks before the dose is further decreased. In some embodiments, the daily dose of OCS e.g., prednisone is maintained at 0.75 mg/kg/day for at least 2 weeks, then maintained at 0.5 mg/kg/day for at least 2 weeks, then maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 2 weeks. In some embodiments, the daily dose of OCS e.g., prednisone is maintained at 0.75 mg/kg/day for at least 2 weeks, then maintained at 0.5 mg/kg/day for at least 2 weeks, then maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 8 weeks. In some embodiments, each daily dose is maintained for two weeks, optionally provided that CIDP symptoms do not worsen.

After maintenance at 0.1 mg/kg/day for at least 2 weeks, the daily dose of OCS e.g., prednisone may optionally be further reduced by 2.5 mg every two weeks for one or more two-week periods. In some embodiments, the daily dose of OCS e.g., prednisone is maintained at 0.1 mg/kg/day for at least 8 weeks before it is further reduced by 2.5 mg every two weeks for one or more two-week periods. In some embodiments, the daily dose of OCS e.g., prednisone is decreased by 2.5 mg every two weeks for one or more two-week periods until the daily dose of OCS e.g., prednisone is zero (i.e., discontinuation of corticosteroid therapy). In some embodiments, each daily dose is maintained for two weeks, optionally provided that CIDP symptoms do not worsen.

Alternatively, after maintenance at 0.1 mg/kg/day for at least 2 weeks, the daily dose of OCS e.g., prednisone may optionally be further reduced by 1 mg every week for one or more one-week periods. In some embodiments, the daily dose of OCS e.g., prednisone is maintained at 0.1 mg/kg/day for at least 8 weeks before it is further reduced by 1 mg every week for one or more one-week periods. In some embodiments, the daily dose of OCS e.g., prednisone is decreased by 1 mg every week for one or more one-week periods until the daily dose of OCS e.g., prednisone is zero (i.e., discontinuation of corticosteroid therapy). In some embodiments, each daily dose is maintained for one week, optionally provided that CIDP symptoms do not worsen.

### Diagnosis

In certain embodiments, the methods described herein may be used to assist with diagnosis of CIDP. As described elsewhere herein, CIDP is a highly heterogenous condition and diagnosis is challenging; misdiagnosis occurs at high frequency. The results presented herein demonstrating efficacy for efgartigimod in the treatment of CIDP, establish CIDP as an autoimmune disease responsive to treatment with FcRn antagonists. The methods described herein may be used to assist with diagnosis of CIDP on the basis that patients suspected of having CIDP (e.g., according to standard diagnostic criteria) and treated according to any of the embodiments described herein may be confirmed as positive for CIDP if they exhibit a positive response to treatment. A positive response to treatment may be measured as control of disease activity, partial remission or complete remission. A positive response to treatment may be measured as an improvement or stabilisation of symptoms as measured using any of the assessment methods described herein. In some embodiments, methods described herein wherein the FcRn antagonist is efgartigimod are used to assist with diagnosis of CIDP.

### F. Drug Products

The instant disclosure provides drug products containing efgartigimod and hyaluronidase approved for treatment of CIDP. Methods of treating CIDP comprising administering a drug product containing efgartigimod and hyaluronidase are also provided. Use of a drug product containing efgartigimod and hyaluronidase for the treatment of CIDP is also provided.

In some embodiments, the drug product is formulated for subcutaneous administration, optionally over approximately 30 to 90 seconds. In some embodiments, the drug product is administered in a total volume of about 5.6 mL. In some embodiments, the drug product is administered in a total volume of 5.6 mL. In some embodiments, the drug product is provided in a single-dose vial for subcutaneous administration via a winged infusion set.

In some embodiments, the drug product is provided in a single-dose vial at a concentration of 180 mg efgartigimod/2000 units hyaluronidase per mL. In some embodiments, the drug product is provided in a single-dose vial containing about 180 mg/mL efgartigimod, about 2,000 units/mL recombinant human hyaluronidase (rHuPH20), about 1.4 mg/mL L-histidine, about 2.2 mg/mL L-histidine hydrochloride monohydrate, about 1.5 mg/mL L-methionine, about 0.4 mg/mL polysorbate 20, about 5.8 mg/mL sodium chloride, and about 20.5 mg/mL sucrose, at a pH of about 6.0.

In some embodiments, the drug product is provided in a single-dose vial containing 180 mg/mL efgartigimod, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, and 20.5 mg/mL sucrose, at a pH of about 6.0.

In some embodiments, the drug product is provided in a single-dose vial containing 5.6 mL of a liquid formulation comprising 1,008 mg efgartigimod, 11,200 units rHuPH20, 7.8 mg histidine, 12.3 mg L-histidine hydrochloride monohydrate, 8.4 mg methionine, 2.2 mg polysorbate 20, 32.5 mg sodium chloride, 114.8 mg sucrose, and water for injection, USP, at a pH of 6.0.

In some embodiments, the drug product is provided in a single-dose vial containing 5.6 mL of a liquid formulation comprising 1,008 mg efgartigimod and 11,200 Units of rHuPH20, and each mL of vial solution contains histidine (1.4 mg), L-histidine hydrochloride monohydrate (2.2 mg), methionine (1.5 mg), polysorbate 20 (0.4 mg), sodium chloride (5.8 mg), sucrose (20.5 mg), and water for injection, USP, at a pH of 6.0.

In some embodiments, the drug product is provided in a single-dose vial containing 180 mg/mL efgartigimod, 2,000 units/mL recombinant human hyaluronidase (rHuPH20), 1.4 mg/mL histidine, 2.2 mg/mL L-histidine hydrochloride monohydrate, 1.5 mg/mL methionine, 0.4 mg/mL polysorbate 20, 5.8 mg/mL sodium chloride, 20.5 mg/mL sucrose, and water for injection, USP, in a total volume of 5.6 mL, at a pH of 6.0.

In some embodiments, the drug product is a reference product.

In some embodiments, the drug product is reference listed on the basis of efgartigimod administered at a dose of 1000 mg. In some embodiments, the drug product is reference listed on the basis of efgartigimod administered at a dose of 1008 mg. In some embodiments, the drug product is reference listed on the basis of hyaluronidase administered at a dose of 11,200 units. In some embodiments, the drug product is reference listed on the basis of once weekly or once every other week administration. In some embodiments, the drug product is reference listed on the basis of once weekly administration. In some embodiments, the drug product is reference listed on the basis of once weekly administration, followed by adjustment to once every other week administration based on clinical evaluation. In some embodiments, the drug product is reference listed on the basis of resumption of once weekly administration in case of worsening of symptoms.

In some embodiments, the drug product is reference listed on the basis of the first 5 injections must be administered either by or under the supervision of a health care professional and subsequent treatment should be administered by a healthcare professional or may be administered at home by a patient or caregiver after adequate training in the subcutaneous injection technique.

In some embodiments, the drug product is approved for administration to an adult human patient. In some embodiments, the drug product has an approved indication for treatment of CIDP in adult human patients with active disease despite treatment with corticosteroids or immunoglobulins.

In some embodiments, the drug product induces an ECI response in 66.5% of a population of CIDP patients who received 1008 mg/11,200 units of efgartigimod PH20 once weekly. In some embodiments, the median time to initial confirmed ECI in the population of CIDP patients is 43 days from first administration of the drug product. In some embodiments, up to 40% of the CIDP patients in the population of CIDP patients had an ECI response four weeks from first administration of the drug product. In some embodiments, 25% of the CIDP patients in the population of CIDP patients showed clinically relevant improvement in at least one of the following: aINCAT score, I-RODS, or mean grip strength, within 9 days of first administration of the drug product. In some embodiments, the population of CIDP patients is 322 CIDP patients. In some embodiments, the patients in the population of CIDP patients who received the drug product remained relapse-free significantly longer than the patients in the population of CIDP patients who received placebo. In some embodiments, the patients in the population of CIDP patients who received the drug product demonstrated a 61% risk reduction for deterioration compared to the patients in the population of CIDP patients who received placebo. In some embodiments, 69% of the CIDP patients in a population of CIDP patients who received the drug product showed evidence of improvement during two consecutive measurements following administration of the FcRn antagonist. In some embodiments, the evidence of improvement is selected from aINCAT improvement ≥1 point, I-RODS improvement ≥4 points, or mean grip strength improvement ≥8 kPa. In some embodiments, the CIDP patients in the population of CIDP who received the drug product experienced a longer time to clinical deterioration compared to the CIDP patients in the population of CIDP patients who received placebo. In some embodiments, the clinical deterioration is an increase of ≥1 point in aINCAT score. In some embodiments, the clinical deterioration is an increase in aINCAT score of ≥1 points during two consecutive measurements. In some embodiments, the clinical deterioration is an increase in aINCAT score of ≥2 points. In some embodiments, the longer time to clinical deterioration is demonstrated by a hazard ratio of 0.394. In some embodiments, the longer time to clinical deterioration is statistically significant.

In some embodiments, the drug product induces a hypersensitivity reaction selected from the group consisting of anaphylaxis and hypotension leading to syncope. In some embodiments, anaphylaxis or hypotension leading to syncope occurs during or within 1 hour of administration of the drug product. In some embodiments, the drug product is contraindicated in patients with serious hypersensitivity to efgartigimod alfa products, to hyaluronidase, or to any excipients in the drug product.

In some embodiments, the drug product induces an infusion-related reaction. In some embodiments, the infusion-related reaction is one or more of the following: hypertension, chills, shivering, thoracic pain, abdominal pain, and back pain. In some embodiments, one or more subsequent doses of the drug product are administered with close clinical observation, slower infusion rates, and pre-medications when a mild-to-moderate infusion-related reaction occurs during administration of the drug product.

In some embodiments, the label for the drug product includes a contraindication in patients with serious hypersensitivity to efgartigimod alfa products, to hyaluronidase, or to any excipients in formulations thereof. In some embodiments, the label for the drug product includes a warning for hypersensitivity reactions selected from anaphylaxis and hypotension leading to syncope. In some embodiments, the label for the drug product includes a warning for infusion-related reactions selected from hypertension, chills, shivering, thoracic pain, abdominal pain, and back pain. In some embodiments, the label for the drug product states to initiate appropriate therapy when a severe infusion-related reaction occurs during administration of the product. In some embodiments, the label for the drug product states that patients may be rechallenged with close clinical observation, slower infusion rates, and pre-medications when a mild-to-moderate infusion-related reaction occurs during administration of the product.

In some embodiments, the drug product induces anti-efgartigimod antibodies in 2% of subjects in a population of 117 CIDP patients following treatment with the drug product. In some embodiments, the drug product induces anti-efgartigimod antibodies in 6% of subjects in a population of 317 CIDP patients following treatment with the drug product. In some embodiments, the drug product induces neutralizing anti-efgartigimod antibodies in 0.3% of subjects in a population of 317 CIDP patients following treatment with the drug product.

In some embodiments, the label for the drug product includes data demonstrating an ECI response in 66.5% of a population of CIDP patients who received 1008 mg/11,200 units of efgartigimod PH20 once weekly. In some embodiments, the label for the drug product includes data demonstrating the median time to initial confirmed ECI in the population of CIDP patients is 43 days from first administration of the drug product. In some embodiments, the label for the drug product includes data demonstrating up to 40% of the CIDP patients in the population of CIDP patients had an ECI response four weeks from first administration of the drug product. In some embodiments, the label for the drug product includes data demonstrating 25% of the CIDP patients in the population of CIDP patients showed clinically relevant improvement in at least one of the following: aINCAT score, I-RODS, or mean grip strength, within 9 days of first administration of the drug product. In some embodiments, the population of CIDP patients is 322 CIDP patients. In some embodiments, the label for the drug product includes data demonstrating the patients in the population of CIDP patients who received the drug product remained relapse-free significantly longer than the patients in the population of CIDP patients who received placebo. In some embodiments, the label for the drug product includes data demonstrating the patients in the population of CIDP patients who received the drug product demonstrated a 61% risk reduction for deterioration compared to the patients in the population of CIDP patients who received placebo. In some embodiments, the label includes data demonstrating improvement at two consecutive visits in 69% of a population of CIDP patients who received 1008 mg/11,200 units of the drug product once weekly up to 12 weeks. In some embodiments, the improvement is aINCAT improvement ≥1 point, I-RODS improvement ≥4 points, or mean grip strength improvement ≥8 kPa. In some embodiments, the label includes data demonstrating that CIDP patients who received the drug product experienced a longer time to clinical deterioration compared to the CIDP patients who received placebo. In some embodiments, the clinical deterioration is an increase of 1 point in aINCAT score. In some embodiments, the clinical deterioration is an increase in aINCAT score of 1 points during two consecutive measurements. In some embodiments, the clinical deterioration is an increase in aINCAT score of 2 points. In some embodiments, the longer time to clinical deterioration is demonstrated by a hazard ratio of 0.394. In some embodiments, the longer time to clinical deterioration is statistically significant. In some embodiments, the label states that patients who received the drug product experienced a longer time to clinical deterioration (i.e., increase of ≥1 point in aINCAT score) compared patients who received placebo, which was statistically significant, as demonstrated by a hazard ratio of 0.394 [95% CI (0.253; 0.614) p<0.0001]. In some embodiments, the label for the drug product includes a combination of any of the features described above or elsewhere herein.

In some embodiments, prior to treatment with efgartigimod PH20, the population of CIDP patients comprises treatment naïve patients, patients receiving immunoglobulin, and patients receiving corticosteroids.

In some embodiments, the population of CIDP patients comprises patients with CIDP that is unstable and refractory to treatment (CIDP Disease Activity Score (CDAS) 5B and 5C) prior to treatment with efgartigimod PH20, and patients with CIDP that is stable on existing treatment (CDAS 3 and 4) prior to treatment with efgartigimod PH20. In some embodiments, the population of CIDP patients are characterized as having disability that is mild to severe prior to treatment with efgartigimod PH20. In some embodiments, the population of CIDP patients are characterized as having an INCAT score of 2-9 prior to treatment with efgartigimod PH20. In some embodiments, the population of CIDP patients are characterized as having an IRODS of 11-61 prior to treatment with efgartigimod PH20. In some embodiments, the population of CIDP patients are characterized as having a mean grip strength of 1-120 kPa prior to treatment with efgartigimod PH20. In some embodiments, the population of CIDP patients are characterized as having a mean INCAT score of 5 prior to treatment with efgartigimod PH20. In some embodiments, the population of CIDP patients are characterized as having a mean IRODS of 37 prior to treatment with efgartigimod PH20. In some embodiments, the population of CIDP patients are characterized as having a mean grip strength of 39 kPa prior to treatment with efgartigimod PH20.

Biosimilars and bioequivalents of the drug product described herein are also encompassed by the instant disclosure.

Methods of treating CIDP comprising administering a drug product containing efgartigimod and hyaluronidase, a biosimilar of a drug product containing efgartigimod and hyaluronidase, or a bioequivalent of a drug product containing efgartigimod and hyaluronidase to a subject are also provided. Use of a drug product containing efgartigimod and hyaluronidase, a biosimilar of a drug product containing efgartigimod and hyaluronidase, or a bioequivalent of a drug product containing efgartigimod and hyaluronidase for the treatment of CIDP in a subject is also provided.

In some embodiments, the drug product, biosimilar, or bioequivalent is administered to the subject once weekly or once every other week. In some embodiments, the drug product, biosimilar, or bioequivalent is administered subcutaneously once weekly. In some embodiments, the drug product, biosimilar, or bioequivalent is administered subcutaneously once weekly for at least 4 weeks. In some embodiments, the drug product, biosimilar, or bioequivalent is administered subcutaneously once weekly until the subject demonstrates EC. In some embodiments, the drug product, biosimilar, or bioequivalent is administered once weekly until the subject demonstrates ECI during two consecutive measurements, optionally two consecutive measurements taken about 1 week apart. In some embodiments, the drug product, biosimilar, or bioequivalent is administered subcutaneously once weekly until the subject demonstrates ECI twice in two weeks. In some embodiments, ECI is clinical improvement in one or more of I-RODS, Mean Grip Strength, INCAT, or aINCAT score. In some embodiments, ECI is a clinical improvement in one or more of I-RODS, Mean Grip Strength, or INCAT score.

In some embodiments, the drug product, biosimilar, or bioequivalent is administered subcutaneously once weekly until the subject demonstrates ECMD. In some embodiments, the ECMD is one or more of an increase in aINCAT score of ≥1 points, a decrease in I-RODS of ≥4 points (using the centile metric), or a decrease in mean grip strength of ≥8 kPa in one hand using a handheld vigorimeter. In some embodiments, the ECMD is an increase in aINCAT score of ≥1 points. In some embodiments, the ECMD is an increase in aINCAT score of ≥1 points during two consecutive measurements, optionally two consecutive measurements taken about 1 week apart. In some embodiments, the ECMD is an increase in aINCAT score of ≥1 points twice in two weeks. In some embodiments, the ECMD is an increase in aINCAT score of ≥2 points.

In some embodiments, the drug product, biosimilar, or bioequivalent is first administered subcutaneously once weekly and is subsequently administered subcutaneously once every two weeks based on clinical evaluation. In some embodiments, the clinical evaluation is performed by a healthcare provider. In some embodiments, the drug product, biosimilar, or bioequivalent is first administered subcutaneously once weekly and is subsequently administered subcutaneously once every two weeks when ECI is demonstrated in the subject. In some embodiments, once weekly subcutaneous administrations of the drug product, biosimilar, or bioequivalent are resumed upon worsening of symptoms. In some embodiments, the drug product, biosimilar, or bioequivalent is administered subcutaneously over approximately 30 to 90 seconds.

In some embodiments, the drug product, biosimilar, or bioequivalent is first administered either by or under the supervision of a healthcare professional for at least 5 administrations. In some embodiments, subsequent administrations of the drug product, biosimilar, or bioequivalent are given by a healthcare professional or by a patient or caregiver.

In some embodiments, the subject has active disease despite treatment with corticosteroids or immunoglobulins.

In some embodiments, the methods or uses described herein further comprise monitoring the subject for a hypersensitivity reaction selected from the group consisting of anaphylaxis and hypotension leading to syncope; and initiating an appropriate measure to mitigate the hypersensitivity reaction when detected.

In some embodiments, the methods or uses described herein further comprise monitoring the subject for an infusion-related reaction; and initiating an appropriate measure when a severe infusion-related reaction is detected. In some embodiments, the infusion-related reaction comprises one or more of the following: hypertension, chills, shivering, thoracic pain, abdominal pain, and back pain.

In some embodiments, the subject is a human, such as, for example, an adult human.

### EXAMPLES

The invention will be further understood with reference to the following non-limiting examples.

### Example 1: A Phase 2 Trial to Investigate the Efficacy, Safety, and Tolerability of Efgartigimod PH20 SC in Adult Patients with CIDP

### 1.1 Protocol Summary

### 1.1.1 Clinical Trial Rationale

An unmet medical need exists for an efficacious treatment of CIDP with a favourable safety and tolerability profile and more convenient administration than that provided by current treatments. A weekly subcutaneous (SC) treatment option consisting of efgartigimod PH20 SC administered within a few minutes could offer clinically significant benefits to CIDP patients.

In the ADHERE clinical trial described herein (Protocol number: ARGX-113-1802), patients with CIDP were administered efgartigimod PH20 SC at the dose of 1000 mg. This dose was well-tolerated in the prior Phase 1 trial ARGX-113-1901 in healthy adult subjects and reduced immunoglobulins G (IgGs) to a level associated with clinical benefit in prior trials in patients with an autoimmune disease (i.e., patients with generalized myasthenia gravis [gMG] and patients with primary immune thrombocytopenia [ITP]).

### 1.1.2 Clinical Trial Methodology

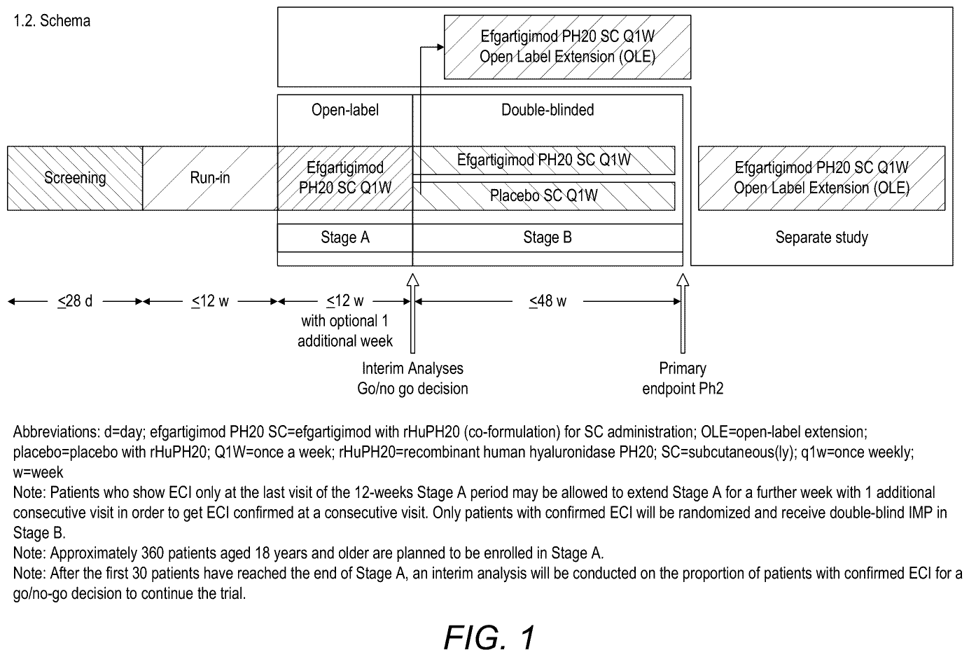

The ADHERE clinical trial was a Phase 2, prospective, multicenter trial on the efficacy, safety, tolerability, immunogenicity, PK, and PD of ‘efgartigimod PH20 SC’ administered SC in patients aged 18 years and older with CIDP. This trial was conducted in 2 stages: An open-label Stage A and a randomized-withdrawal, double-blind, placebo-controlled Stage B. The full Schema is shown in FIG. 1.

During the screening period of a maximum of 28 days, diagnosis of CIDP was confirmed by a CIDP confirmation committee (CCC) and overall eligibility was confirmed by the medical monitor. Eligible patients receiving treatment for CIDP at screening discontinued that treatment and entered the run-in period for up to 12 weeks until ECMD was confirmed (i.e., observed during a trial visit), at which time eligible patients entered Stage A at baseline (DIA).

Eligible patients who were treatment-naïve or discontinued treatment with corticosteroids and/or intravenous immunoglobulin (IVIg) or subcutaneous immunoglobulin (SCIg) at least 6 months prior to screening (who were considered equal to treatment-naïve patients) entered Stage A directly, if during screening documented evidence for worsening on the total aINCAT disability score, further referred to as “aINCAT score,” within 3 months prior to screening was available compared to previous aINCAT score within 6 months prior to screening.

Patients eligible for Stage A received open-label investigational medicinal product (IMP) as weekly SC administrations of efgartigimod PH20 SC for up to 12 weeks (with optional 1 additional week for confirmation of ECI, if needed), with a minimum of 4 administrations. During Stage A, patients were monitored for ECI.

Patients who fulfilled the criteria for ECI at 2 consecutive visits (confirmed ECI status), were rolled over to the randomized-withdrawal, placebo-controlled Stage B. Patients who did not have confirmed ECI were not eligible for Stage B and ended the trial after performing the safety follow-up visit 28 days after the last administration of the IMP.

After the first 30 patients reached the end of Stage A, an interim analysis was conducted on the proportion of patients with confirmed ECI using the exact (Clopper-Pearson) 90% 1-sided lower confidence interval (CI) for a go/no-go decision to continue the trial. During the interim analysis, the trial continued without any interruption, neither for trial enrollment nor for visits or treatments of patients participating in any trial period.

Patients for whom ECI in Stage A was confirmed, entered the double-blind, randomized-withdrawal Stage B and were randomized at Stage B baseline (D1B) in a 1:1 ratio to receive weekly IMP consisting of efgartigimod PH20 SC or placebo. The randomized-withdrawal Stage B lasted for up to 48 weeks.

All patients randomized to the double-blind, randomized-withdrawal Stage B were dosed with IMP weekly but returned to the clinic in 4-week intervals (every 4 weeks). Administration of randomized IMP treatment only occurred after completion of all indicated assessments. Patients were discharged from the center if there were no safety concerns in the opinion of the investigator. Between the trial visits in Stage B, patients were given the option to come to the trial center weekly for drug administration or were provided with home nurse service for IMP injecting. Upon agreement with the investigator, site personnel, and the patient, the most suitable solution was provided. Administration of IMP always occurred within a time window of ±2 days with respect to the pre-planned date of administration.

Patients completing Week 48 and in the opinion of the investigator benefitting from trial treatment at Week 48 or patients having an event of worsening on the aINCAT score of 1 point or a worsening on the aINCAT score of ≥2 points compared to Stage B baseline (for the latter, no confirmation is needed) were allowed to roll over to the open-label extension (OLE) trial when they were receiving IMP. Patients completing Week 48 who were not rolled over to the OLE trial as well as patients with an early discontinuation, had a safety follow-up visit 28 days after the last IMP administration, stopped the trial, and were treated as considered appropriate by the investigator.

When eighty-eight (88) events had been observed for the primary endpoint analysis of Stage B, then the trial was stopped. Patients in Stage A and Stage B performed an early discontinuation visit and patients receiving IMP were given the opportunity to continue efgartigimod PH20 SC treatment in the OLE trial. Additionally, at the time of the 88th event, patients in run-in were also given the option to roll over to the OLE trial after performing an early discontinuation visit. Patients who did not roll over to the OLE trial, attended a follow-up visit 28 days after the last IMP dose.

From Stage A onwards, patients received training for self-administration of IMP, which was foreseen in the OLE trial (not in the ARGX-113-1802 trial).

### 1.1.3 Objectives

### 1.1.3.1 Stage a (Open-Label, Efgartigimod PH20 SC; 4-12 Weeks [+1 Optional Additional Week])

### Primary Objective

To assess the activity of efgartigimod PH20 SC (efgartigimod co-formulated with recombinant human hyaluronidase PH20 [rHuPH20]) based on the percentage of patients classified as treatment responders.

### Secondary Objectives

To assess the time to clinical improvement.
To determine the treatment effect of efgartigimod PH20 SC based on clinical functional assessments of motor function and muscle strength.
To assess the short-term safety and tolerability of efgartigimod PH20 SC.
To assess the pharmacokinetics (PK) of efgartigimod PH20 SC.
To assess the pharmacodynamic (PD) effect of efgartigimod PH20 SC.
To assess the immunogenicity of efgartigimod and rHuPH20.
To assess the EuroQol 5 dimensions and 5 levels health-related quality-of-life questionnaire (EQ-5D-5L).

### Exploratory Objectives

To evaluate additional patient-reported outcomes (PROs) (including additional patient-reported quality of life and satisfaction with treatment).
To evaluate biomarkers of CIDP disease activity.
To evaluate the association between biomarkers and clinical outcomes.
To explore the composite endpoint validation analysis.

### 1.1.3.2 Stage B (Double-Blind, Randomized-Withdrawal, Efgartigimod PH20 SC or Placebo; Up to 48 Weeks)

### Primary Objective

To determine the efficacy of efgartigimod PH20 SC compared to placebo based on the time needed for the occurrence of the first evidence of clinical deterioration.

### Secondary Objectives

To determine the efficacy of efgartigimod PH20 SC compared to placebo based on clinical functional assessments of disease disability and motor function and muscle strength.
To assess the safety and tolerability of efgartigimod PH20 SC.
To assess the PK of efgartigimod PH20 SC.
To assess the PD effect of efgartigimod PH20 SC.
To assess the immunogenicity of efgartigimod and rHuPH20.
To assess the EQ-5D-5L.

### Exploratory Objectives

To evaluate additional PROs (including additional patient-reported quality of life and satisfaction with treatment).
To evaluate biomarkers of CIDP disease activity.
To evaluate the association between biomarkers and clinical outcomes.
To explore the composite endpoint validation analysis.

### 1.1.4 Maximum Duration of Patient Involvement in the Trial

After a screening period of up to 28 days and a run-in period up to 12 weeks, the total duration of IMP dosing was up to 61 weeks and included 2 treatment stages:
Stage A was up to 12 weeks (with optional 1 additional week) open-label treatment of efgartigimod PH20 SC; and
Stage B was up to 48 weeks of double-blind treatment with IMP (efgartigimod PH20 SC or placebo).

Follow-up period for patients not rolling over into the extension trial or for patients who prematurely discontinued IMP, was 28 days after the last dose of IMP.

The maximum total trial duration was 80 weeks with a maximum of 61 weeks on IMP:
Screening period: up to 28 days
Run-in period: up to 12 weeks (not required for treatment-naïve patients)
Stage A: 4-12 weeks (with optional 1 additional week)
Stage B: up to 48 weeks
Follow-up period: 28 days after the last dose of IMP for patients who did not enroll into the OLE trial ARGX-113-1902.

### 1.1.5 Endpoints

### 1.1.5.1 Populations for Analyses

Safety population: The Stage A safety population (SAF-A) included all patients who received at least 1 dose of IMP in Stage A. The Stage B safety population (SAF-B) included all patients who received at least 1 dose of IMP in Stage B.

Modified intent-to-treat (mITT) population: The mITT population included all randomized patients who received at least 1 dose of IMP in Stage B.

Per protocol (PP) population: The PP population comprised all Stage B patients in the mITT population for whom no major protocol deviation was reported.

### 1.1.5.2 Stage A

### Primary Endpoint:

Percentage of patients with confirmed ECI.

### Secondary Endpoints:

Evidence of clinical activity:
   Time to initial confirmed ECI.
   Change from Stage A baseline (DIA) over time in:
      aINCAT score
      MRC sum score
      24-item I-RODS disability scores
      TUG score
      Mean grip strength assessed by Martin vigorimeter.
Safety:
   Exposure adjusted occurrence of treatment-emergent adverse events (TEAEs) and serious adverse events (SAEs) by system organ class (SOC) and preferred term (PT);
   Incidence of clinically significant laboratory abnormalities.
PK profile:
   Pre-dosing efgartigimod serum concentrations over time during Stage A.
PD profile:
   Changes of serum IgG levels (total IgG) over time during Stage A.
Immunogenicity:
   Percentage of patients with and titers of binding antibodies (BAb) toward efgartigimod and/or rHuPH20 during Stage A.
   Presence of neutralizing antibodies (NAb) against efgartigimod and titers of NAb against rHuPH20 during Stage A.
PRO:
   Changes from DIA in EQ-5D-5L over time during Stage A.

### Exploratory Endpoints:

### Additional Patient-Reported Outcomes

Change from Stage A baseline (DIA) over time during Stage A in:
   Brief Pain Inventory Short Form (BPI-SF)
   (9-item) Treatment Satisfaction Questionnaire for Medication (TSQM-9)
   Rasch-transformed-Fatigue Severity Scale (RT-FSS)
   Hospital Anxiety and Depression Scale (HADS)
   Patient Global Impression of Change (PGIC)

### Biomarker Analysis

Change from Stage A baseline (DIA) over time during Stage A in autoantibody levels, which may include but are not limited to anti-GM1, anti-LM-1, anti-NF-155, anti-CNTN1, anti-Caspr-1 antibody levels, and anti-myelinated nerve antibodies.

### 1.1.5.3 Stage B

### Primary Endpoint:

Time to first aINCAT deterioration compared to Stage B baseline.

Note: Time to first aINCAT deterioration was defined by the time from first dose of double-blind IMP to the first aINCAT score increase of 1 point compared to Stage B baseline, if the deterioration was confirmed at a consecutive visit 3-7 days after the first aINCAT score increase of 1 point. For patients with an increase of 2 or more points on the aINCAT score compared to Stage B baseline, no confirmation was required.

### Secondary Endpoints:

Clinical Efficacy:
   Time to CIDP disease progression.
      Note: Time to CIDP disease progression is defined by the time from first dose of double-blind IMP to the first I-RODS score decrease ≥4 points compared to Stage B baseline using the centile metric.
   Percentage of patients with improved functional level compared to Stage B baseline as measured by an increase in the 24-item I-RODS score up to Week 48.
   Change from Stage B baseline over time in:
      aINCAT score
      MRC Sum score
      24-item I-RODS disability scores
      TUG score
      Mean grip strength assessed by Martin vigorimeter.
   Time to 10% decrease in the 24-item I-RODS during Stage B.
Safety:
   Incidence of TEAEs and SAEs by SOC and PT during Stage B.
   Incidence of clinically significant laboratory abnormalities during Stage B.
PK Profile:
   Pre-dosing efgartigimod serum concentrations over time during Stage B.
PD Profile:
   Changes of serum IgG levels (total IgG) over time during Stage B.
Immunogenicity:
   Percentage of patients with and titers of BAb toward efgartigimod and/or rHuPH20 during Stage B.
   Presence of NAb against efgartigimod and titers of NAb against rHuPH20 during Stage B.
PRO:
   Changes from Stage B baseline in EQ-5D-5L over time during Stage B.

### Exploratory Endpoints:

### Additional Patient-Reported Outcomes

Change from Stage B baseline over time during Stage B in:
   Brief Pain Inventory Short Form (BPI-SF)
   (9-item) Treatment Satisfaction Questionnaire for Medication (TSQM-9)
   Rasch-transformed-Fatigue Severity Scale (RT-FSS)
   Hospital Anxiety and Depression Scale (HADS)
   Patient Global Impression of Change (PGIC)

### Biomarker Analysis

Change from Stage B baseline over time during Stage B in autoantibody levels, which may include but are not limited to anti-GM1, anti-LM-1, anti-NF-155, anti-CNTN1, anti-Caspr-1 antibody levels, and anti-myelinated nerve antibodies.

### 1.2 Trial Design

### 1.2.1 Overall Design

The ADHERE trial was a Phase 2, prospective, multicenter trial to investigate the efficacy, safety, tolerability, PK, and PD of efgartigimod PH20 SC in patients aged 18 years and older with CIDP. This trial was conducted in 2 stages: An open-label Stage A and a randomized-withdrawal Stage B.

After a screening period for all patients and a run-in period for applicable patients (not for treatment-naïve patients), all patients entered the open-label Stage A at baseline (D1A) and received weekly trial treatment (efgartigimod PH20 SC) for up to 12 weeks (with optional 1 additional week). Within this period, upon ECI which was confirmed at 2 consecutive visits, patients were moved into the randomized-withdrawal, placebo-controlled Stage B and were randomized at Stage B baseline (D1B) in a 1:1 ratio to efgartigimod PH20 SC or placebo. The randomized-withdrawal Stage B lasted up to 48 weeks.

The trial included the following periods:
Screening period: up to 28 days;
Run-in period: up to 12 weeks (not for treatment-naïve patients);
Stage A: up to 12 weeks (with optional 1 additional week) of open-label treatment of efgartigimod PH20 SC (weekly trial visits);
Stage B: up to 48 weeks of double-blind, randomized-withdrawal treatment of efgartigimod PH20 SC or of placebo (trial visits once every 4 weeks); and
Follow-up: 28 days after the last dose of IMP if the patient did not intend to roll over to the open-label extension (OLE) trial ARGX-113-1902.

Total trial duration for each patient was up to 80 weeks with a maximum of 61 weeks on IMP. The trial schema is provided in FIG. 1.

### 1.2.1.1 Screening Period

During the screening period of a maximum of 28 days, the investigator's diagnosis of CIDP was reviewed by a CIDP confirmation committee (CCC), and overall eligibility confirmed by the medical monitor. To enter the study, participants had to be diagnosed with CIDP as confirmed by a CCC and by the medical monitor.

The CCC was composed of neurologists with at least 10 years' experience in the diagnosis of CIDP and a documented track record of at least 100 diagnoses of CIDP. The CCC ensured that the CIDP trial population included in this trial fulfilled the official EFNS/PNS 2010 diagnostic criteria (Van den Bergh et al., 2010). These were used to define probable or definitive progressing or relapsing forms of CIDP in which both sensory symptoms and limited motor functions, or limited motor functions only had to be present for patients to qualify for the trial.

### 1.2.1.2 Run-In Period

After the screening period and depending on the type of permitted previous treatments for CIDP, eligible patients started a run-in period. The run-in period lasted up to 12 weeks. The run-in period included the following, depending on the prior or current treatment of the patient at screening:
1. Patients, who were treatment-naïve for treatments for CIDP were allowed to skip the run-in period, if there was documented evidence for worsening on the aINCAT score within 3 months prior to screening compared to previous aINCAT score within 6 months prior to screening;
(Patients that were not treated with pulsed monthly or daily corticosteroids, IVIg or SCIg for at least 6 months prior to screening were considered as equal to treatment-nave patients).
2. Patients, who were being treated with IVIg or SCIg during screening stopped treatment with IVIg or SCIg at the start of the run-in period (i.e., RI-V1);
3. Patients, who were being treated with pulsed corticosteroids (IV or oral) or daily oral corticosteroid treatment ≤10 mg during screening stopped treatment with corticosteroids at the start of the run-in period (i.e., RI-V1).

Patients were monitored by collecting the grip strength, I-RODS score, MRC sum scoring, aINCAT score, and the TUG test data. Regular scheduled visits at the trial site were planned every 4 weeks.

Patients received appropriate training and instructions to assess every week the disability status by means of I-RODS and grip strength. The patient's I-RODS score and the mean grip strength were captured, calculated, and transferred to the electronic case report form (eCRF). If the patient observed any of the following signals of deterioration, a trial visit was arranged within 5 working days for an assessment by a physician:
an I-RODS deterioration by ≥4 points (using the centile metric), and/or
a mean grip strength deterioration by ≥8 kPa in 1 hand using the handheld vigorimeter.

At the trial site, the evaluating physician assessed if deterioration could be confirmed by fulfilling the criteria for ECMD.

Any patient showing ECMD at the trial site entered Stage A immediately. If the patient was not providing evidence of ECMD by the end of the run-in period, then he/she was recorded as a run-in failure.

### 1.2.1.3 Stage A

All patients with ECMD started Stage A with the first administration of open-label IMP at Stage A baseline (D1A). After completing all pretreatment baseline assessments, the patients received weekly SC administrations of IMP, consisting of efgartigimod PH20 SC, for up to 12 weeks (with optional 1 additional week), with a minimum of 4 SC injections of IMP.

The procedures that were completed at D1A and at the other trial visits are provided in the Schedule of Activities (SoA) in Table 9. Each patient remained in Stage A and returned weekly to the trial site as described in the SoA until ECI relative to D1A was identified by the evaluating investigator at 2 consecutive visits.

An interim analysis of results was performed after the first 30 patients in Stage A reached the end of Stage A (EOSA). During the interim analysis, the trial continued without any interruption, neither for trial enrollment nor for visits or treatments of patients participating in any trial period.

Patients who did not show ECI at 2 consecutive visits during Stage A were classified as non-responders. Non-responders ended the trial and attended a follow-up visit 28 days after the last administration of IMP, and were treated by the investigator as considered appropriate.

All patients with confirmed ECI were randomized to 1 of the 2 treatment arms (SC administration of efgartigimod PH20 SC or placebo) and started Stage B. Randomization was performed on the same day as the confirmation of ECI. Patients who showed ECI only at the last visit of Stage A (i.e., 1 week after A-V12) were allowed to extend Stage A for a further week with 1 additional consecutive visit. If at this visit ECI was observed for the first time, it was followed by a consecutive visit 1 week later. The reason for this possible extension of Stage A with 1 week was to get ECI confirmed at a consecutive visit 1 week later (and, if ECI was confirmed, to randomize the patient in Stage B). In the case that ECI was not confirmed after an additional week of Stage A, an EOSA visit was performed and the patient ended the trial (after a follow-up visit 28 days following the last IMP administration).

During Stage A, IMP was administered at the site after blood samples were taken for laboratory safety, PK, PD, immunogenicity, and/or biomarker analyses and after all assessments needed for determination of ECI. During Stage A, all patients received training for self-administration of IMP (foreseen in the OLE trial).

TABLE 9
1.3.1. SoA for Screening, Run-in, and Stage A

| | | Run-in | | | | | | | | | | | | | | | | | Safety |
|---|---|---|---|---|---|---|---|---|---|---|---|---|---|---|---|---|---|---|---|
| | Screening^{a} | Period | | | | | | | | | | | | | | | | Unsche- | Follow- |
| | ≤28 days | ≤12 weeks^{b} | Stage A: Open-Label Period ≤12 Weeks (With Optional 1 Additional Week) | | | | | | | | | | | | | | | duled^{e} | up^{f} |
| | | | RI-V2 | | | | | | | | | | | | | A-V13c | | | 28 (±3) |
| | | | to | | | | | | | | | | | | | (Addi- | | | Days |
| | | RI-V1 | RI-V4 | A-V1 | A-V2 | A-V3 | A-V4 | A-V5 | AV6^{c} | A-V7^{c} | A-V8^{c} | A-V9^{c} | A-V10^{c} | A-VH^{c} | A-V12^{c} | tional) | | | After |
| | Up to | | | | | | | | | | | | | | | | EOSA/ | | Last IMP |
| | 28 days | | | D1A | D8 | D15 | D22 | D29 | D36^{c} | D43^{c} | D50^{c} | D57^{c} | D64^{c} | D71^{c} | D78^{c} | D85 | ED^{d} | UnchV | Dose |
| | | ±0 | ±2 | ±0 | | | | | | | | | | | | | | | |
| Assessment/Procedure | | days | days | days | ±2 days | | | | | | | | | | | | | | |
| Informed consent | • | | | | | | | | | | | | | | | | | | |
| Demographics | • | | | | | | | | | | | | | | | | | | |
| In-/exclusion criteria | • | • | | • | | | | | | | | | | | | | | | |
| Medical history^{g} | • | | | | | | | | | | | | | | | | | | |
| 12-lead ECGs | • | | | • | | | | | | | | | | | | | • | • | • |
| CDAS | • | | | | | | | | | | | | | | | | | | |
| Physical exam and vital | •^{h} | •ⁱ | •ⁱ | •ⁱ | •ⁱ | •ⁱ | •ⁱ | •ⁱ | •ⁱ | •ⁱ | •ⁱ | •ⁱ | •ⁱ | •ⁱ | •ⁱ | •ⁱ | •ⁱ | •ⁱ | •ⁱ |
| signs | | | | | | | | | | | | | | | | | | | |
| Pregnancy test | •^{j} | •^{k} | •^{k} | •^{k} | | | | •^{k} | | | | •^{k} | | | | | •^{k} | •^{k} | •^{k} |
| Adjusted INCAT score | | • | • | • | • | • | • | • | (•)^{l} | • | (•)^{l} | • | (•)^{l} | • | (•)^{l} | • | • | • | |
| MRC Sum score | | • | • | • | • | • | • | • | (•)^{l} | • | (•)^{l} | • | (•)^{l} | • | (•)^{l} | • | • | • | |
| I-RODS score | | • | • | • | • | • | • | • | • | • | • | • | • | • | • | • | • | • | |
| Mean grip strength | | • | • | • | • | • | • | • | • | • | • | • | • | • | • | • | • | • | |
| TUG test | | • | • | • | • | • | • | • | • | • | • | • | • | • | • | • | • | • | |
| EQ-5D-5L | | | | • | | | | | | | | | | | | | • | • | |
| BPI-SF | | | | • | | | | | | | | | | | | | • | • | |
| TSQM-9 | | •^{m} | | | | | | | | | | | | | | | • | • | |
| RT-FSS | | | | • | | | | | | | | | | | | | • | • | |
| HADS | | | | • | | | | | | | | | | | | | • | • | |
| PGIC | | | | | • | | | | | | | | | | | | • | • | |
| Suicidality | • | • | • | • | • | • | • | • | • | • | • | • | • | • | • | • | • | • | • |
| assessment [symbol] | | | | | | | | | | | | | | | | | | | |
| Blood & urine clinical | •^{o} | | | • | • | • | • | • | | | | | | | | | • | • | • |
| laboratory safety tests | | | | | | | | | | | | | | | | | | | |
| HIV, HBV, HCV, and | • | | | | | | | | | | | | | | | | | •^{p} | |
| tuberculosis testing | | | | | | | | | | | | | | | | | | | |
| Blood sampling for PK | | | | •^{w} | • | • | • | • | • | • | • | • | • | • | • | • | • | • | • |
| and PD analysis^{q} | | | | | | | | | | | | | | | | | | | |
| Blood sampling for | | •^{w} | | •^{w} | | | | | | | | | | | | | • | • | |
| biomarker analysis [symbol] | | | | | | | | | | | | | | | | | | | |
| IMP self-administration | | | | • | • | • | • | •^{t} | •^{t} | •^{t} | •^{t} | •^{t} | •^{t} | •^{t} | •^{t} | •^{t} | | | |
| training [symbol] | | | | | | | | | | | | | | | | | | | |
| IMP admistration | | | | • | • | • | • | •^{u} | •^{u} | •^{u} | •^{u} | •^{u} | •^{u} | •^{u} | •^{u} | •^{u} | | | |
| (SC) [symbol] | | | | | | | | | | | | | | | | | | | |
| IMP accountability [symbol] | | | | • | • | • | • | •^{u} | •^{u} | •^{u} | •^{u} | •^{u} | •^{u} | •^{u} | •^{u} | •^{u} | • | | |

| | |
|---|---|
| Adverse events^{v} | 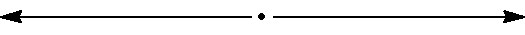 |
| Concomitant therapies^{g,v} | 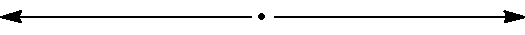 |

Footnotes are on next page
ADA = anti-drug antibodies;
AE = adverse event;
BPI-SF = Brief Pain Inventory Short Form;
CDAS = CIDP disease activity status;
CIDP = chronic inflammatory demyelinating polyneuropathy;
DIA = baseline of Stage A;
DIB = baseline of Stage B;
ECG = electrocardiogram;
ECI = evidence of clinical improvement;
ECMD = evidence of clinically meaningful deterioration;
ED = early discontinuation;
EOSA = end of Stage A;
EQ-5D-5L = EuroQol 5 dimensions and 5 levels health-related quality-of-life questionnaire;
HADS = Hospital Anxiety and Depression Scale;
HBV = hapatitis B virus;
HCV = hepatitis C virus;
HIV = human immunodeficiency virus;
ICF = informed consent form;
IgG = immunoglobulin G;
IMP = investigational medical product;
INCAT = inflammatory neuropathy cause and treatment;
I-RODS = Inflammatory-Rasch-built Overall Disability Scale;
OLE = open-label extension;
PD = pharmacodynamic(s);
PGIC = Patient Global Impression of Change;
PK = pharmacokinetic(s);
rHuPH20 = recombinant human hyaluronidase PH20;
RT-FF = Rasch-transformed-Fatigue Severity Scale;
MRC = Medical Research Council;
SAE = serious adverse event;
SC = subcutaneous(ly);
TSQM = Treatment Satisfaction Questionnaire for Medication;
TUG = timed up and go;
UNS = unschedules;
V = visit;
W = week.
^{a}Within 28 days before the first day of run-in (visit RI-1). If needed a medical monitor can authorize the extension of the screening period. For treatment-na [symbol] ve patients screening can occur within 28 days before DIA, if during screening documented evidence for worsening on the adjusted INCAT score within 3 months prior to screening is available compared to previous adjusted INCAT score within 6 months prior to screening.
^{b}site visit every 4 weeks. RI-V1: day 1 of run-in; RI-V2; run-in week 4 (±2 days); RI-V3; run-in week (±2 days), and RI-V4; run-in week 12 (±2 days). A patient showing ECMD will enter Stage A immediately. Patients will receive appropriate training and instructions to access every week the disability status by means of I-RODS and grip strength. If the patient does not have evidence of ECMD by the end of the run-in period, then he/she should be recorded as a run-in failure.
^{c}Weekly administration of efgartigimod PH20 SC (±2 days). Patients, who show ECI only after the 12^{th} IMP administration (ie, 1 week after the A-V12 visit), may be allowed to extend Stage A for a further week with an additional visit (the visit at which ECI is observed for the first time will then be A-V13) and patients will need to come back 1 week later (ie, 1 week after A-V13) to confirm ECI.
^{d}The assessments in this visit are for patients who end Stage A (ie, EOSA visit; for patients with or without confirmed (ECI) as well as for patients who discontinue the trial prematurely (ie, ED visit). Patients with confirmed ECI can continue the trial in Stage B (in this case, the patients will have a combination of the EOSA visit and the Baseline Stage B [D1B] visit [see Section 1.3.2]).
Note: Patients who end Stage A with confirmed ECI and will go to Stage B, should have had at least 4 IMP administrations during Stage A.
Note: Patients with ECI only after 12 IMP administrations in stage A, may be allowed to extend Stage A for 1 more week (in order to determined if ECI is confirmed or note) with an additional consecutive visit (see footnote above).
^{e}The investigator can decide which of the assessments need to be performed at each unschedules visit (refer to Section 5.1.5).
^{f}Only applicable for patients who do not continue in Stage B and for patients who prematurely discontinued IMP in case IMP was stopped less than 28 days before the last trial visit.
^{g}At screening, all available vaccination history will be captured as part of prior medication. For vaccines where multiple doses or boosters are recived, only the most recent one must be recorded. Any vaccination received during the trial (from screening onwards) will be enterned as concomitant medication.
Note: For patients who provide separate consent, the titers of produced vaccination antibiotics will be measured using retained blood samples (left over blood samples taken for other tests) during the trial.
^{h}Includes physical examination, height, weight, semi-supine blood pressure and heart rate, and body temperature.
ⁱIncludes physical examination, weight, semi-supine blood pressure and heart rate, and body temperature.
^{j}A serum pregnancy test will be conducted at screening.
^{k}Urine pregnancy tests will be conducted every 4 weeks during the trial and at the follow-up visit.
^{l}These tests are only required for confirmation of improvement (for patients with improvement in the previous visit).
^{m}At the first visit of the run-in period (RI-V1), the TSQM-9 should be completed based on the treatment taken before the start of the trial.
ⁿAt screening, suicidality will be assessed with the Columbia-suicide severity rating scale (C-SSRS) (See Section 9.2). During the trial, suicidality will be assessed by specifically answering 1 question from the PHQ-9 depression questionnaire (see Section 9.15).
^{o}At screening, total IgG will be measured (by central laboratory as part of the blood chemistry tests) to determine eligibility.
^{p}Serology ca be retested only during the screening period.
^{q}For PK, pre-dose (within 2 hours prior to start of IMP administration at the trial visits) samples will be taken. At these time points, blood samples will also be taken for PD analysis. PD analysis includes the measurement of total IgG, IgG testing must not be perfomed locally.
^{r}Blood samples for immunogenicity testing will be taken pre-dose at the trial visits to measure ADA against eligartigimod (measured in serum) and antibodies against rHuPH20 (measured in plasma).
^{s}Optional blood samples will be taken for autoantibodies testing (serum) and serum-sample storage for future testing.
^{t}Patients will be trained to self-administer IMP (foreseen in the OLE trial; not in the ARGX-113-1802 trial) during the first 4 visits when IMP is administered; thereafter, training for self-administration is optional (only if needed).
^{u}IMP will be administered weekly at the site in Stage A (IMP administration will be after blood samples have been taken for laboratory safety, PK, PD, immunogenicity, and/or biomarker analyses and after all assessments needed for determination of ECI). Patients will receive a minimum of 4 IMP administrations in Tage A. Note that IMP in Stage A will not be administered if ECI is confirmed at the visit (and the patient has received at least 4 IMP administrations). When ECI is confirmed (ie, ECI observed at the second consecutive
visit), the patient will be randomized and receive the first double-blind IMP administration in Stage B (ie, the visit at which ECI is confirmed will be a combination of the EOSA visit and Stage B baseline [D1B] visit [see Section 1.3.2]).
^{v}AEs and intake of concomitant medication(s) will be monitored coninously from signing the ICP until the last trial-related activity. In case of early discontinuation, any AEs/SAEs should be assessed for 30 days following the ED visit or until satisfactory resolution or stabilization.
^{w}Blood samples may also be used to cross-validate the PK, PD, biomarker, and ADA assays in CIDP matrix (serum and plasma). [symbol]
indicates data missing or illegible when filed

### 1.2.1.4 Stage B

Evaluations at screening, during the run-in period (visit RI-V1 to visit RI-V4), and confirmation at Stage A baseline (D1A; visit A-V1) before the first treatment in Stage A and the treatment response during Stage A were used to determine each patient's eligibility for randomization in Stage B at Stage B baseline (D1B).

Patients with ECI during Stage A were randomized at Stage B baseline (D1B) in a 1:1 ratio to receive weekly SC administrations of efgartigimod PH20 SC or placebo.

All patients randomized to the double-blind, randomized-withdrawal Stage B were dosed with IMP weekly but returned to the clinic at 4-week intervals. At each of these visits, specific procedures, as specified in the Schedule of Activities (SoA) in Table 10, were performed.

Patients were discharged from the trial center after all planned procedures for that visit according to the SoA were performed and if no safety concerns existed in the opinion of the investigator.

The trial was stopped when 88 events were observed for the primary endpoint analysis of Stage B. At this point, patients in Stage A and Stage B performed an early discontinuation visit and patients receiving IMP were given the opportunity to continue efgartigimod PH20 SC treatment in the OLE trial (in this case the early discontinuation visit of the ARGX-113-1802 trial could coincide with the roll over visit). Patients who did not roll over to the OLE trial, attended a follow-up visit 28 (±3) days after the last IMP dose.

Patients were given the opportunity to continue efgartigimod PH20 SC treatment in an OLE trial—ARGX-113-1902—if any of the following 3 conditions occurred:
1. The patient experienced clinical deterioration (i.e., worsening in aINCAT score) during Stage B.
2. The patient completed week 48 visit of Stage B without any clinical deterioration.
3. Recording of the 88th event during Stage B and the patient was receiving IMP in either Stage A or Stage B.

An event is a worsening of the aINCAT score in any of patients treated during Stage B.

Additionally, at the time of the 88th event, patients in run-in were also given the option to roll over to the OLE trial after performing an early discontinuation visit.

During Stage B, patients received further training for self-administration of IMP (foreseen in the OLE trial, not in the ARGX-113-1802 trial) as specified in the SoA in Table 10. Patients who had clinical deterioration or who completed the week 48 visit also had the possibility to enter the safety follow-up period and complete the trial without rolling over to the OLE trial.

### 1.2.1.5 Follow-Up

A follow-up visit was carried out 28 days after the last dose of IMP (in case of early discontinuation from IMP or the trial), or in case a patient did not meet the criteria for ECI in Stage A [non-responder]) or 28 days after the last dose in Stage B if the patient did not roll over to the OLE trial ARGX-113-1902.

TABLE 10
1.3.2. SoA for Stage B and Follow-up

| | B-V1 | B-V2 | B-V3 | B-V4 | B-V5 | B-V6 | B-V7 | B-V8 | B-V9 | B-V10 | B-V11 | B-V12 | B-V13 | | Safety |
|---|---|---|---|---|---|---|---|---|---|---|---|---|---|---|---|
| | D1B^{a} | W4 | W8 | W12 | W16 | W20 | W24 | W28 | W32 | W36 | W40 | W44 | W48/ED^{b} | | Follow- |
| Assessment/Procedure | ±0 days | ±2 days | | | | | | | | | | | | UNS | up^{d} |
| Randomization | • | | | | | | | | | | | | | | |
| 12-lead ECGs | • | | | • | | | • | | | • | | | • | • | • |
| Physical exam and vital | • | • | • | • | • | • | • | • | • | • | • | • | • | • | • |
| signs [symbol] | | | | | | | | | | | | | | | |
| Pregnancy test^{f} | • | • | • | • | • | • | • | • | • | • | • | • | • | • | • |
| Adjusted INCAT score | • | • | • | • | • | • | • | • | • | • | • | • | • | • | |
| MRC sum score | • | • | • | • | • | • | • | • | • | • | • | • | • | • | |
| I-RODS score | • | • | • | • | • | • | • | • | • | • | • | • | • | • | |
| Mean grip strength | • | • | • | • | • | • | • | • | • | • | • | • | • | • | |
| TUG test | • | • | • | • | • | • | • | • | • | • | • | • | • | • | |
| EQ-5D-5L | • | | | • | | | • | | | • | | | • | • | |
| BPI-SF | • | | | • | | | • | | | • | | | • | • | |
| TSQM-9 | • | | | • | | | • | | | • | | | • | • | |
| RT-FSS | • | | | • | | | • | | | • | | | • | • | |
| HADS | • | | | • | | | • | | | • | | | • | • | |
| PGIC | | | | | | | | | | | | | • | • | |
| Suicidality assessment | • | • | • | • | • | • | • | • | • | • | • | • | • | • | • |
| Blood & urine safety | • | | | • | | | • | | | • | | | • | • | • |
| tests | | | | | | | | | | | | | | | |
| Blood sampling for PK | • | • | • | • | • | • | • | • | • | • | • | • | • | • | • |
| and PD analysis^{g} | | | | | | | | | | | | | | | |
| Blood sampling for | • | • | • | • | • | • | • | • | • | • | • | • | • | • | • |
| immunogenicity | | | | | | | | | | | | | | | |
| analysis^{h} | | | | | | | | | | | | | | | |
| Blood sampling for | • | | | • | | | • | | | • | | | • | • | |
| biomarker analysisⁱ | | | | | | | | | | | | | | | |
| IMP self- | • | • | • | • | • | • | • | • | • | • | • | • | | | |
| administration | | | | | | | | | | | | | | | |
| training^{j} | | | | | | | | | | | | | | | |
| IMP administration | • | • | • | • | • | • | • | • | • | • | • | •^{k} | | | |
| (SC)^{k} | | | | | | | | | | | | | | | |
| IMP dispensation | • | • | • | • | • | • | • | • | • | • | • | •^{k} | | | |
| IMP accountability | • | • | • | • | • | • | • | • | • | • | • | • | • | | |

| | |
|---|---|
| Adverse events^{l} | 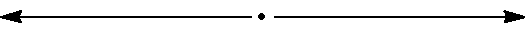 |
| Concomitant therapies^{l,m} | 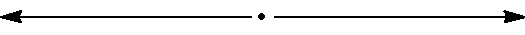 |

ADA = anti-drug antibody;
AE = adverse event;
BPI-SF = Brief Pain Inventory Short Form;
D18 = baseline of Stage B;
ECG = electrocardiogram;
ECI = evidence of clinical improvement;
ED = early discontinuation;
EQ-5D-5L = EuroQol 5 dimensions and 5 levels health-related quality-of-life questionnaire;
HADS = Hospital Anxiety and Depression Scale;
HBV = hepatitis B virus;
HCV = hapatitis C virus;
HIV = human immunodeficiency virus;
ICF = informed consent form;
IgG = immunoglobulin G;
IMP = investigational medical product;
INCAT = inflammatory neuropathy cause and treatment;
I-RODS = Inflammatory-Rasch-built Overall Disability Scale;
OLE = open-label extension;
PD = pharmacodynamic(s);
PGIC = Patient Global Impression of Change;
PK = pharmacokinetic(s);
rHuPH20 = recombinant human hyaluronidase PH20;
RT-FSS = Rasch-transformed-Fatigue Severity Scale;
MRC = Medical Research Council;
SAE = serious adverse event;
SC = subcataneous(ly);
TSQM = Treatment Satisfaction Questionnaire for Medication;
TUG = timed up and go;
UNS = unscheduled;
V = visit;
W = week.
^{a}DIB will occur at the same visit, at which ECI has been confirmed in Stage A. There is no need to schedule an additional visit for DIB. All assessments are performed prior to IMP administration.
^{b}The assessments in this visit are for patients who end Stage B as well as for patients who discontinue the trial prematurely (ie, ED visit). Patients who will roll over to the open-label extension trial, will have a ombination this last visit in the ARGX-113-1802 trial and the first visit of the open-label extension trial ARGX-113-1902.
^{c}The investigator can decide which of the indicated assessments need to be performed at each unscheduled visit (refer to Section 5.1.5).
^{d}This visit is not applicable for patients who roll over to the open-label extension trial and for patients who prematurely discontinued IMP in case IMP was stopped less than 28 days before the last trial visit.
^{e}Includes physical examination, weight, semi-supine blood pressure and heart rate, and body temperature.
^{f}Urine pregnancy test every 4 weeks and at the follow-up viti.
^{g}For PK, pre-dose (within 2 hours prior to start of IMP administration at the trial visits) samples will be taken. At these time points, blood samples will also be taken for PD analysis. PD analysis includes the measurement of total IgG. IgG testing must not be performed locally.
^{h}Blood samples for immunogenicity testing will be taken pre-dose at the trial visits to measure ADA against efgartigimod (measured in serum) and antibodies against rHuPH20 (measured in plasma).
ⁱOptional blood samples will be taken for autoantibodies testing (serum) and serum-sample storagte for future testing.
^{j}Patients will be trained (refreshment training), if needed, to self-administer IMP (foreseen in the OLE tria; not in the ARGX-113-1802 trial).
^{k}IMP will be administered at the site at the scheduled visits. This will be after blood samples have been taken for laboratory safety, PK, PD, immunogenicity, and/or biomarker analyses. For IMP administrations between the scheduled visits, the patient can choose between nurse home visits or return to the trial site for the SC injections
only. The last planned IMP administration is at week 47.
^{l}AEs and intake of concomitant medication(s), will be monitored continuously from signing the ICF until the last trial-related activity. In case of early discontinuation, any AEs/SAEs should be assessed for 30 days following the ED visit or until satisfactory resolution or stabilization.
^{m}Any vaccination received during the trial (from screening onwards) will be entered as concomitant medication.
Note: For patients who provide separate consent, the titers of produced vaccination antibodies will be measured using retained blood samples (left over blood samples taken for other tests) during the trial. [symbol]
indicates data missing or illegible when filed

### 1.2.2 Scientific Rationale for Trial Design

The ADHERE trial design used some key elements from the PATH trial (van Schaik et al., 2018). A treatment period (Stage A) was available for all patients who showed evidence of disease activity during the run-in period.

The run-in period had 2 purposes:
Selecting for patients with evidence of clinical disease activity (enrichment design);
Ensuring that both, the half-life of the previous drug (if applicable) had elapsed and the clinical effect would not jeopardize the efficacy analysis of the trial.

The pre-randomization observation period on treatment (Stage A) could be of any length between 4 and 13 weeks. The post-withdrawal observation period (i.e., for patients in the placebo group during Stage B) in this trial had a maximum duration of 48 weeks. Careful attention was paid to procedures for monitoring patients and assessing trial endpoints to ensure that patients failing on an assigned treatment were rapidly identified.

The advantages of this trial design were that individuals receiving the experimental intervention continued to do so only if they responded, whereas individuals receiving placebo did so only until their symptoms returned. An additional advantage of this design was to study the long-term efficacy or safety (withdrawal effect).

The trial design allowed for the collection of information on the onset of therapeutic effect as well as information if the effect continues after withdrawal from medication or if there is a rebound phenomenon (placebo group). Furthermore, the trial also generated data of the placebo response in such a population that is of interest for further clinical development.

Any difference that emerges between the group receiving continued treatment and the group randomized to placebo in Stage B demonstrates the effect of the active treatment.

All eligible patients started the participation in the trial through an open-label period (Stage A), in which they all received active treatment. Such a choice was perceived necessary for the purpose to offer to all patients with active disease a minimal treatment with efgartigimod PH20 SC, even for those patients who would be randomized to the placebo arm after this period (in Stage B). Stage A had a variable duration at minimum 4 weeks up to 12 weeks (with optional 1 additional week), during which the patient was treated with weekly SC administrations until clinical improvement had been achieved and maintained in 2 concurrent visits. This variable duration was chosen to achieve the maximal IgG reduction, which would maximize the duration of effect. A maximum number of injections was set at 12 (or 13 in case of an optional additional week), in line with clinical practice for other therapies (3 months).

As introduction into the placebo arm, an initial open-label treatment with efgartigimod PH20 SC (Stage A) was foreseen for all patients. This allowed patients to receive active treatment before potential randomization to placebo (during Stage B), which made the design more acceptable within the current context of highly effective treatment standards in this debilitating disease. In addition, Stage A followed by randomization to the placebo arm allowed to establish how long the treatment effect would last before clinical evidence of disease activity appeared again. In this way, the trial allowed to evaluate through biomarker analysis, if a personalized treatment schedule might be an option in clinical practice.

Patients with clinical deterioration, as measured by an increase of the aINCAT score at any time during Stage B, were offered the opportunity to roll over to a separate OLE trial with weekly dosing of efgartigimod PH20 SC. Double blinding of the original treatment assignment was maintained throughout the trial, even at the time of withdrawal.

The possibility to roll over to the OLE trial made the trial attractive for patients who took an advantage from open-label treatment during Stage A and knew that the effective open-label treatment would be available for them again (in the OLE trial) despite the option to be randomized to placebo and to potentially deteriorate in the randomized Stage B of the trial.

All efficacy, safety, and quality-of-life assessments used in this trial were standard, i.e., widely used (including in CIDP trials) and recognized as reliable, accurate, and relevant.

The biomarker analysis was exploratory, and the biomarkers were selected for evaluating treatment effects of efgartigimod PH20 SC on the physiopathology in CIDP and for evaluating correlation between biomarkers and clinical outcomes. The evaluation of these biomarkers might allow to drive a development toward a personalized treatment approach with efgartigimod PH20 SC, with a prognostic value for the likelihood of the occurrence of clinical deterioration, or a predicting value to establish the likelihood of treatment response. These are nowadays however, not described as accepted surrogate biomarkers. For this reason, this evaluation had an exploratory nature. The total human IgG levels themselves are only a measure on how long the decrease of IgG levels is ongoing after suspension of treatment with efgartigimod PH20 SC. This was a relevant reference marker for those patients, who after the induction period were randomized to the placebo arm.

### 1.2.3 Rationale for Dose

A dose of 1000 mg efgartigimod PH20 SC was selected for both Stage A and Stage B. Based on population PK/PD modeling, this dose of efgartigimod PH20 SC (weekly administration) was predicted to be comparable to 10 mg/kg efgartigimod weekly administered as IV infusion with respect to effect on IgG levels. The efgartigimod 10 mg/kg IV dose:
1. Resulted in transient clinical efficacy in a Phase 2 trial in ITP patients and a prolonged clinical effect in a Phase 2 trial in MG patients following 4 weekly infusions.
2. Was shown to result in close to saturated PD effect:
   Dosing higher or more frequently than weekly is not expected to result in an improved PD effect (i.e., further lowering of autoantibodies) and/or clinical effect and may be associated with a less optimal risk/benefit ratio. Dosing lower is expected to result in a lower PD effect and thus is likely to result in a less consistent and/or incomplete clinical response, which is undesirable given the serious and chronic manifestations of CIDP. Therefore, weekly dosing is favorable until patients are in a stable clinical condition.
3. Demonstrated a favorable safety profile in Phase 2 trials in MG and ITP patients.

In addition, based on favorable phase 3 results in patients with MG, efgartigimod IV at a dose of 10 mg/kg has been approved in the United States (US) and in the EU (in both regions for AChR-Ab seropositive patients) and in Japan (for patients who do not have sufficient response to steroids or nonsteroidal immunosuppressive therapies).

A study in healthy participants demonstrated that after 4 weekly injections of efgartigimod PH20 SC 1000 mg, the pattern of total IgG level reduction over time was comparable to that observed after 4 weekly infusions of efgartigimod IV 10 mg/kg.

rHuPH20, as Hylenex, has been approved in the US. Coformulations of rHuPH20 with other active ingredients have been approved in the US and the EU (e.g., HERCEPTIN HYLECTA/Herceptin SC, RITUXAN HYCELA/MabThera SC, HYQVIA/HyQvia, respectively), with a rHuPH20 concentration of 2000 U/mL for a SC injection volume in the range of 5 to 13.4 mL.

### 1.2.4 Rationale for Randomization and Blinding

Stage A was open-label with administration of efgartigimod PH20 SC. In Stage B, patients were randomized to double-blind SC efgartigimod PH20 SC or placebo in a 1:1 ratio. Patients were stratified according to their prior CIDP medication and the decrease of aINCAT score during Stage A by the following:
1: Prior CIDP medication:
   Treatment-naïve;
   Pulsed corticosteroid treatment or oral corticosteroids equivalent to prednisolone/prednisone 10 mg/day;
   IVIg or SCIg treatment.
2: aINCAT score:
   No change in aINCAT score during Stage A;
   aINCAT score decrease of ≥1 point during Stage A.

Except for unblinding for safety reasons, Stage B was double-blind to treatment assignment during the entire randomized-withdrawal period, even if the patient withdrew from the trial or entered the OLE trial ARGX-113-1902. The treatment that each patient received was not disclosed to the investigator, investigational site staff, patient, sponsor, or the sponsor's designated contract research organization (CRO).

### 1.2.5 Roll Over to Open-Label Extension (OLE) Trial

Patients who were receiving IMP, and in the opinion of the investigator were benefitting from trial treatment at week 48 or who, while on treatment, experienced clinical deterioration during Stage B (i.e., worsening in aINCAT score) were offered the option to roll over to a long-term, single-arm, OLE trial, during which they would be treated weekly with SC efgartigimod PH20 SC. The dose and frequency used in the OLE is the same as was given to patients enrolled to Stage A of this trial.

Patients who were receiving IMP in Stage A or Stage B, or were in run-in at the moment of recording of the 88th event in the trial, performed an early discontinuation visit and were offered the option to roll over to the OLE trial, as the ARGX-113-1802 trial stopped at this moment. An event is a worsening of the aINCAT score in any of the patients treated during Stage B.

### 1.3 Trial Population

An informed consent form (ICF) was signed by each patient prior to any trial-related assessment. Prior to giving informed consent, patients were instructed not to participate in any other clinical trial that involved a medical intervention or collection of data until the completion of the current trial.

Patients were classified as eligible if all of the inclusion criteria and none of the exclusion criteria were fulfilled, and eligibility CIDP diagnosis was confirmed by a CCC and by the medical monitor.

### 1.3.1 Inclusion Criteria

Patients were eligible to be included in the trial only if all of the following criteria applied:
1. Ability to understand the requirements of the trial, provide written informed consent (including consent for the use and disclosure of research-related health information), willingness and ability to comply with the trial protocol procedures (including required trial visits).
2. Male or female patient aged 18 years or older, at the time of signing the informed consent.
3. Diagnosed with probable or definite CIDP according to criteria of the European Federation of Neurological Societies/Peripheral Nerve Society (EFNS/PNS, 2010), progressing or relapsing forms.
4. CIDP Disease Activity Status (CDAS) score ≥2 at screening (Gorson et al., 2019).
5. An INCAT score ≥2 at the first run-in visit (RI-V1; for patients entering run-in) or Stage A baseline (A-V1; for treatment-naïve patients with documented evidence for worsening on the total aINCAT disability score within 3 months prior to screening). Patients with an INCAT score of 2 at trial entry were required to have this score exclusively from the leg disability score; for patients with an INCAT score of ≥3 at trial entry, there was no specific requirements for arm or leg scores.
6. Fulfilling any of the following treatment conditions:
   Currently (i.e., within the last 6 months) treated with pulsed corticosteroids, oral corticosteroids equivalent to prednisolone/prednisone ≤10 mg/day, and/or IVIg or SCIg, and the patient was willing to discontinue this treatment at the first run-in visit (RI-V1); OR
   Without previous treatment (treatment-naïve); OR
   Treatment with corticosteroids and/or IVIg or SCIg discontinued at least 6 months prior to screening.
Note: Patients not treated with monthly or daily corticosteroids, IVIg or SCIg for at least 6 months prior to screening were considered as equal to treatment-naïve patients.
7. Women of childbearing potential who have a negative pregnancy test at screening and a negative urine pregnancy test up to Stage A baseline (D1A).
Women of childbearing potential were required to use an acceptable method of contraception from signing the ICF until the date of the last dose of IMP.

### 1.3.2 Exclusion Criteria

Patients were excluded from the trial if any of the following criteria applied:
1. Pure sensory atypical CIDP (EFNS/PNS definition).
2. Polyneuropathy of other causes, including the following:
   Multifocal motor neuropathy
   Monoclonal gammopathy of uncertain significance with anti-myelin-associated glycoprotein immunoglobulin M (IgM) antibodies
   Hereditary demyelinating neuropathy
   Polyneuropathy, organomegaly, endocrinopathy, monoclonal protein, and skin change syndromes
   Lumbosacral radiculoplexus neuropathy
   Polyneuropathy most likely due to diabetes mellitus
   Polyneuropathy most likely due to systemic illnesses
   Drug- or toxin-induced polyneuropathy.
3. Any other disease that could better explain the patient's signs and symptoms.
4. Any history of myelopathy or evidence of central demyelination.
5. Current or past history (within 12 months of screening) of alcohol, drug or medication abuse.
6. Severe psychiatric disorder (such as severe depression, psychosis, bipolar disorder), history of suicide attempt, or current suicidal ideation that in the opinion of the investigator could create undue risk to the patient or could affect adherence with the trial protocol.
7. Patients with clinically significant active or chronic uncontrolled bacterial, viral, or fungal infection at screening, including patients who test positive for an active viral infection at screening with:
   Active Hepatitis B Virus (HBV): serologic panel test results indicative of an active (acute or chronic) infection;
   Active Hepatitis C Virus (HCV): serology positive for HCV-Ab;
   Human Immunodeficiency Virus (HIV) positive serology associated with an Acquired Immune Deficiency Syndrome (AIDS)-defining condition or with a cluster of differentiation 4 (CD4) count ≤200 cells/mm3.
8. Total IgG level <6 g/L at screening.
9. a. Treatment with the following:
   Within 3 months (or 5 half-lives of the drug, whichever is longer) before screening: plasma exchange or immunoadsorption, any concomitant Fc-containing therapeutic agents or other biological, or any other investigational product;
   Within 6 months before screening: rituximab, alemtuzumab, any other monoclonal antibody, cyclophosphamide, interferon, tumor necrosis factor-alpha inhibitors, fingolimod, methotrexate, azathioprine, mycophenolate, any other immunomodulating or immunosuppressive medications, and oral daily corticosteroids ≥10 mg/day.
   Patients using IVIg, SCIg, pulsed corticosteroids, and oral daily corticosteroids <10 mg/day can be included.
   Patients who (intend to) use prohibited medications and therapies during the trial.
10. a. Pregnant and lactating women and those intending to become pregnant during the trial.
11. Patients with any other known autoimmune disease that, in the opinion of the investigator, would interfere with an accurate assessment of clinical symptoms of CIDP.
12. a. Patients who received a live-attenuated vaccine fewer than 28 days before screening. Receiving an inactivated, sub-unit, polysaccharide, or conjugate vaccine any time before screening is not exclusionary.
13. Patients who have a history of malignancy unless deemed cured by adequate treatment with no evidence of recurrence for ≥3 years before the first IMP administration. Patients with the following cancer can be included anytime:
   Adequately treated basal cell or squamous cell skin cancer,
   Carcinoma in situ of the cervix,
   Carcinoma in situ of the breast, or
   Incidental histological finding of Prostate cancer (TNM [tumor, nodes, and metastases classification] stage T1a or T1b).
14. Patients who previously participated in a trial with efgartigimod and have received at least 1 administration of IMP.
15. Patients with known medical history of hypersensitivity to any of the ingredients of IMP.
16. Patients with clinical evidence of other significant serious disease or patients who underwent a recent or have a planned major surgery, or any other reason which could confound the results of the trial or put the patient at undue risk.

### 1.3.3 Prohibited Medications

Medication for treatment of CIDP was continued during the screening period but discontinued from the start of the run-in period (RI-V1) onwards:
Intravenous or subcutaneous immunoglobulin therapy;
Pulsed corticosteroids and oral daily corticosteroids ≤10 mg/day.

The following medications or treatments were not permitted from signing the ICF at screening during the entire trial:
Plasmapheresis;
Total lymphoid irradiation;
Any other IgG therapy;
Any stem cell-based therapy, including bone marrow transplantation like autologous stem cell transplant (ASCT) and allogenic cells transplants;
Any cytokine or anti-cytokine therapy;
Systemic corticosteroids, including oral, for any other indication than CIDP;
Cyclophosphamide, interferon, tumor necrosis factor-alpha inhibitors, fingolimod, methotrexate, azathioprine, mycophenolate, or any other immunomodulating or immunosuppressive medications or procedures;
Rituximab, alemtuzumab, or any other monoclonal antibody for immunomodulation;
Any investigational drug or experimental procedure;
Live-attenuated vaccines
Note: Receiving an inactivated, sub-unit, polysaccharide, or conjugate vaccine any time before screening was not prohibited.
Use of complementary therapies, including traditional Chinese medicines and herbal remedies, containing any of the following: naturally derived glucocorticoids; medication with clinical evidence-based immunosuppressive, immunomodulatory, peripheral or central nervous system effects; or procedures (e.g., acupuncture) for any neurological condition within 4 weeks or 5 half-lives (whichever is longer) prior to IMP dosing and agreed not to use during the trial.

### 1.4 Study Interventions

The investigational medicinal products (IMPs) used in this study were:
Open-label Stage A—efgartigimod PH20 SC
Double-blind Stage B—efgartigimod PH20 SC and placebo.

Efgartigimod is a modified human IgG1 Fc fragment with increased affinity to human FcRn.

rHuPH20 is an enzyme used to increase the dispersion and absorption of co-administered therapeutics when administered SC.

Efgartigimod and rHuPH20 are co-formulated and were administered as a single SC injection.

The efgartigimod PH20 SC formulation was provided in a vial at a concentration of 165 mg/mL or 180 mg/mL for efgartigimod and 2000 U/mL for rHuPH20 (also referred to as ARGX-113/rHuPH20). Each dose of efgartigimod PH20 SC included 1000 mg efgartigimod. Note that there was a transition period during which both formulations of efgartigimod PH20 SC (with efgartigimod at a concentration of 165 mg/mL or 180 mg/mL) were used. After this transition period, all patients received the efgartigimod PH20 SC formulation with efgartigimod at the higher concentration of 180 mg/mL. The formulation with the higher concentration of efgartigimod (180 mg/mL) reduced the dosing volume for each SC injection.

Placebo was vehicle (with 2000 U/mL of rHuPH20) provided in a vial as ready SC formulation. For both efgartigimod PH20 SC formulations (with efgartigimod at a concentration of 165 mg/mL or 180 mg/mL), a corresponding placebo was available containing placebo drug product at the same volume and in the same vial as the active drug product.

Fixed doses of efgartigimod PH20 SC or PBO PH20 SC were administered in the abdominal skin area.

### 1.5 Trial Assessments and Procedures

Each participant was instructed to attend each study visit on the designated days in accordance with the Schedules of Activities as shown in Tables 9 and 10.

1.5.1 Definition of Evidence of Clinically Meaningful Deterioration During the Run-In Period, Evidence of Clinical Improvement During Stage a, and aINCAT Deterioration During Stage B ECMD

ECMD is defined as the fulfilling of any of the following criteria during the run-in period only:
Total aINCAT score increase by ≥1 point from the first visit of the run-in period (RI-V1); and/or
I-RODS decrease by ≥4 points (using the centile metric) from RI-V1; and/or
Mean grip strength decrease by ≥8 kPa in 1 hand using the handheld vigorimeter from RI-V1.
Any patient showing ECMD at the trial site during the run-in period entered Stage A immediately.

### ECI During Stage a for Non-Naïve Patients

Patients who deteriorated in aINCAT score during the run-in period (increase ≥1 point versus the first visit of the run-in period [RI-V1]) could only be entered in Stage B if they showed improvement in aINCAT score during Stage A (decrease ≥1 point versus Stage A baseline [D1A]). These patients entered Stage B at the time the improvement on the aINCAT score was confirmed.

Patients who had no change in aINCAT score during the run-in period and deteriorated on I-RODS and/or grip strength during run-in could be entered in Stage B in the following cases:
When improvement on the aINCAT score (decrease ≥1 point) versus D1A was confirmed.
When no change in aINCAT score was observed during Stage A and:
   When improvement during Stage A on I-RODS (increase ≥4 points versus D1A) was confirmed in case deterioration on only I-RODS (decrease ≥4 points) was observed during run-in.
   When improvement during Stage A on grip strength (increase ≥8 kPa versus D1A) was confirmed in case deterioration on only grip strength (decrease ≥8 kPa) was observed during run-in.
   When improvement during Stage A on either I-RODS (increase 4 points versus D1A) or grip strength (increase ≥8 kPa versus D1A) was confirmed in case deterioration was observed on both I-RODS (decrease ≥4 points) and grip strength (decrease ≥8 kPa) during run-in.

### ECI During Stage a for Naïve Patients

Naïve patients, who did not enter the run-in period and who showed an improvement during Stage A of at least 1 grade on the aINCAT score (i.e., decrease by ≥1 point) compared to D1A, could enter Stage B at the time the improvement on the aINCAT score (decrease ≥1 point) was confirmed.

aINCAT Deterioration

A deterioration of the aINCAT score was defined as an increase (i.e., worsening) of 1 point on the aINCAT score compared to Stage B baseline which was confirmed at a consecutive visit 3-7 days after the first aINCAT score increase of 1 point. For patients with an increase of 2 or more points on the aINCAT score compared to Stage B baseline, no confirmation was required.

### 1.5.2 Efficacy Assessments

Time points for all efficacy assessments are shown in the Schedule of Activities (see Tables 9 and 10). During all visits, the efficacy assessments were performed first, before any other trial-specific procedures, except for the ICF process during screening.

1.5.2.1 aINCAT Disability Score

The INCAT score is a 10-point scale that covers the functionality of legs and arms and has been successfully used to measure treatment effects in various CIDP trials. Scores for arm disability range from 0 (“No upper limb problems”) to 5 (“Inability to use either arm for any purposeful movement”), and scores for leg disability range from 0 (“Walking not affected”) to 5 (“Restricted to wheelchair, unable to stand and walk a few steps with help”), see Table 11 below.

TABLE 11

| | |
|---|---|
| Arm | 0 = No upper limb problems |
| disability | 1 = Symptoms, in 1 or both arms, not affecting the ability |
| (05) | to perform any of the following functions: Doing all zips and |
| | buttons; washing or brushing hair, using a |
| | knife and fork together, handling small coins |
| | 2 = Symptoms, in 1 arm or both arms, affecting but not |
| | preventing any of the above-mentioned functions |
| | 3 = Symptoms, in 1 arm or both arms, preventing 1 or 2 of |
| | the above-mentioned functions |
| | 4 = Symptoms, in 1 arm or both arms, preventing 3 or all |
| | of the functions listed, but some purposeful movements |
| | still possible |
| | 5 = Inability to use either arm for any purposeful movement |
| Leg | 0 = Walking not affected |
| disability | 1 = Walking affected, but walks independently outdoors |
| (05) | 2 = Usually uses bilateral support (stick, single |
| | crutch, 1 arm) to walk outdoors |
| | 3 = Usually uses bilateral support (sticks, crutches, |
| | 2 arms) to walk outdoors |
| | 4 = Usually uses wheelchair to travel outdoors, but |
| | able to stand and walk a few steps with help |
| | 5 = Restricted to wheelchair, unable to stand and walk |
| | a few steps with help |

Overall disability = Arm disability + Leg disability

The INCAT (total) score is the sum of these 2 scores and ranges from 0 to 10. For the aINCAT score, changes in the function of the upper limbs from 0 (normal) to 1 (minor symptoms) or from 1 to 0 will not be recorded as deterioration or improvement because these changes are not considered clinically significant.

There were 3 different baseline INCAT scores during the trial, at the beginning of each stage. Each baseline INCAT score is always the total INCAT score (not the aINCAT score, which was used for post-baseline assessments):
Baseline run-in period=Total INCAT score at the first visit of the run-in period (RI-V1)
Baseline Stage A=Total INCAT score at D1A (i.e., the first visit of Stage A [A-V1])
Baseline Stage B=Total INCAT score at D1B (i.e., the first visit of Stage B [B-V1])
The baseline total INCAT score is calculated as the sum of the arm and leg sub score, without adjustment of the arm sub score.

For the post-baseline visits, the aINCAT score calculation was based on the baseline of the corresponding stage.

### 1.5.2.2 MRC Sum Score

The 6-point MRC sum score evaluates motor strength/weakness:
0=complete paralysis
1=minimal contraction
2=Active movement with gravity eliminated
3=Weak contraction against gravity
4=Active movement against gravity and resistance
5=Normal strength.

The MRC Sum Score is evaluated bilaterally on 6 muscle groups of upper and lower limbs in order to obtain a summed score between 0 and 60:
Arm abductors
Elbow flexors
Wrist extensors
Hip flexors
Knee extensors
Foot dorsiflexors.

### 1.5.2.3 I-RODS

The I-RODS is a PRO (patient reported outcome) measure on disability and assesses the limitations of activities and social participation in patients with inflammatory neuropathies like Guillain-Barré syndrome (GBS), CIDP, and gammopathy-related polyneuropathy (MGUSP).

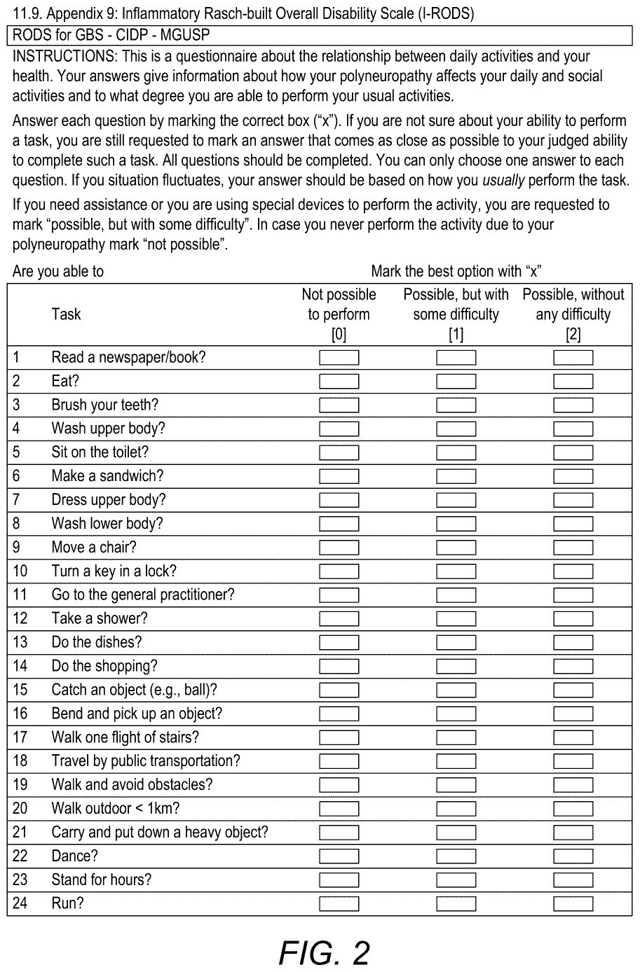

The I-RODS is a 24-item scale, with each item representing a common daily activity that range from very difficult to do, like running or dancing, to very easy to do, like eating or reading a book. Higher scores indicate less disability. The 24-item scale is shown in FIG. 2. The raw sum scores of the I-RODS (range: 0 to 48) were converted to a centile metric score, ranging from 0 (most severe activity and social participation restrictions) to 100 (no activity and social participation limitations).

Although the I-RODS as a PRO can be considered subjective, the scale has shown to correlate well with grip strength, which is a direct measure of muscle strength. When assessed at home, the patient recorded the measurements.

### 1.5.2.4 Mean Grip Strength

A handheld Martin vigorimeter was used for this assessment. Patients performed 3 assessments for each hand in an arbitrary order (with a rest period of approximately 30 seconds between each assessment) always at approximately the same time during the day. The mean grip strength for each hand was used to determine disease activity. When assessed at home, the patient recorded the measurements.

### 1.5.2.5 TUG Test

The TUG test is a simple test used to assess a person's mobility and requires both static and dynamic balance. In the TUG test, the time expended to rise from a chair, walk 3 meters, turn around, walk back to the chair, and sit down is measured. During the test, the person is expected to wear their regular footwear and use any mobility aids that they would normally require.

### 1.5.3 Pharmacokinetics

Blood samples for determination of serum concentrations of efgartigimod were collected from D1A pre-dose onwards and at certain time points up to the follow-up visit as specified in the SoAs. Blood samples were also used to cross-validate the PK assay in CIDP matrix (serum). Concentrations of efgartigimod in serum were determined using a validated assay.

### 1.5.4 Pharmacodynamics

Blood samples for determination of total IgG in serum were taken at several time points during the trial from D1A pre-dose onwards as indicated in the SoAs. Blood samples were also used to cross-validate the PD assays in CIDP matrix (serum).

### 1.5.5 Biomarkers

Blood samples were collected from all patients who provided separate consent for biomarker analysis at the time points provided in the SoAs to assess potential biomarkers in CIDP.

Blood samples were taken in this study for autoantibodies analysis at RI-V1, A-V1, EOSA/ED/D1B, B-V4, B-V7, B-V10, B-V13, UnschV visits. Samples from the RI-V1 (or A-V1 for those who skipped the run-in period) visit were batch analyzed at the end of study and subsequent samples from patients with positive autoantibodies at this visit were tittered and analyzed.

Autoantibody biomarkers analysed in this study included but were not limited to: anti-GM1, anti-LM-1, Anti-NF-155, anti-CNTN1, anti-Caspr-1 antibodies, and anti-myelinated nerve antibodies.

### 1.5.6 Immunogenicity Assessments: Anti-Drug Antibodies (ADA)

Samples for determination of anti-drug antibodies (ADA) against efgartigimod (measured in serum) and antibodies against rHuPH20 (measured in plasma) were taken prior to dosing on Stage A baseline (D1A) and before the SC injection of IMP at several time points as indicated in the SoAs.

Titers of binding antibodies (BAb) and the presence of neutralizing antibodies (NAb) against efgartigimod were measured in serum. Titers of BAb and NAb against rHuPH20 were measured in plasma. NAb were tested for all confirmed positive ADA samples.

### 1.5.7 Additional Patient-Reported Outcome (PRO) Tools

Additional PRO data, associated with medical encounters, were measured at Stage A baseline (D1A) and at several time points during the trial as indicated in the SoAs Participants completed the following PRO assessments at the timepoints listed in the SoA:
EuroQol 5 Dimensions and 5 Levels Health-related Quality-of-Life Questionnaire (EQ-5D-5L) (euroqol.org/eq-5d-instruments/eq-5d-51-about/)
Brief Pain Inventory—Short Form (BPI-SF) (Cleeland and Ryan, 1994).
Treatment Satisfaction Questionnaire for Medication (TSQM): In this trial, TSQM-9 was used (Atkinson et al., 2004; Atkinson et al., 2005; Bharmal et al., 2009)
Rasch-transformed-Fatigue Severity Scale (RT-FSS): In this trial, the abbreviated RT-FSS was used (Van Nes et al., 2009).
Hospital Anxiety and Depression Scale (HADS): (Zigmond et al., 1983; Bjelland et al., 2002)
Patient Global Impression of Change (PGIC): (eprovide.mapi-trust.org/instruments/patient-global-impression-of-change)

### 1.5.8 Additional Clinical Laboratory Tests

TABLE 12
Hematology
Hematology

| | | |
|---|---|---|
| Red blood cell count | Hemoglobin | Hematocrit |
| Mean cell volume | Mean cell | Mean cell |
| | hemoglobin | hemoglobin |
| Red cell distribution width | Platelets | concentration |
| White blood cell count | Lymphocytes count | Monocytes |
| Eosinophils | Basophils | Neutrophils count |

TABLE 13
Coagulation
Coagulation

| | | |
|---|---|---|
| International normalized | Activated partial thromboplastin | Prothrombin |
| ratio | time | time |

TABLE 14
Blood Chemistry Tests
Blood Chemistry Tests

| | | |
|---|---|---|
| Glucose | Creatinine | Total protein^{§} |
| Albumin^{§} | Bilirubin | Alkaline phosphatase |
| Aspartate transferase | Alanine | Gamma glutamyl |
| | transferase | transferase |
| Cholesterol* | High-density | Low-density |
| | lipoprotein | lipoprotein |
| | cholesterol* | cholesterol (LDL)* |
| Total cholesterol/HDL | Triglycerides* | Estimated glomerular |
| ratio* | | filtration rate* |
| high-sensitive C | Blood urea nitrogen | Hemoglobin A1c |
| reactive protein | | |
| Lactate dehydrogenase | Uric acid | Potassium |
| Sodium | Calcium | Serum pregnancy |
| Total immunoglobulin | | test* |
| G (IgG)* | | |

*At screening visit only
^{§}In the exceptional circumstances that using a central laboratory is not possible: Do not measure total protein and albumin after the Stage B baseline visit.
# Chronic Kidney Disease - Improved Prediction Equations (CKD-EPI) will be used for estimating glomerular filtration rate: it will be reported as creatinine clearance.

TABLE 15
Blood Screening for Infections
Blood Screening for Infections

| | | |
|---|---|---|
| HIV^{§} | Viral Hepatitis (HBsAg, anti-HBc | Tuberculosis |
| (Ag/Ab test) | Ab, Anti-HBs, Anti-HCV Ab [HCV | (Quantiferon) |
| | RNA if reactive]) | |

Abbreviations: Ab = antibody; Ag = antigen; HBc = hepatitis B core; HBs = hepatitis B surface; HBsAg = hepatitis B surface antigen; HCV = hepatitis C virus; HIV = human immunodeficiency virus.
^{§}CD4 count to be analyzed in case of positive HIV (see exclusion criterion #7)

TABLE 16
Urine Analysis
Urine Analysis

| | |
|---|---|
| Dipstick: pregnancy, pH, protein, | In case of abnormal dipstick results*, |
| glucose, ketone, bilirubin, | light microscopy: erythrocytes, |
| urobilinogen, blood, nitrite, | leucocytes, casts, and epithelial cells |
| leucocytes, and specific gravity | |

*If urinary tract infection is suspected, investigations (such as microscopy, culture and sensitivity) should be performed locally by the investigator.

### 1.6 Results

ADHERE, the largest pivotal study of CIDP (n=322), recruited a broad population representative of the CIDP population.

The baseline characteristics of the CIDP study population were as follows:
CIDP treatment prior to study:
   Treatment-naive patients (29%, receiving no treatment)
   Patients receiving Ig (51.2%) or corticosteroids (19.6%).
Disease characteristics:
   CIDP that was unstable and refractory to treatment (43.5% CIDP Disease Activity Score (CDAS) 5B and 5C)
   Patients considered stable on existing treatment (37.0% CDAS 3 and 4).
Baseline disability that was mild to severe:
   INCAT score (mean 5 (range 2-9))
   IRODS (mean 37 (11-61))
   Mean Grip Strength (39 kPa (1-120)).

The results were as follows:

### 1.6.1 Stage A

During Stage A, open label weekly efgartigimod PH20 SC was clinically effective with a rapid onset of effect.
67% (214/322) of patients responded (met ECI criteria).
   A sensitivity analysis excluding patients who were ongoing in stage A at the time of study completion showed an overall response rate of 70%.
   Median time to response was approximately 6 weeks; approximately 40% had ECI at week 3 (the earliest timepoint that the criteria could be met).
The majority of patients across all prior CIDP medication subgroups responded to treatment with efgartigimod.
   59% in the prior IVIg group, 78% in prior corticosteroids and 72% in the treatment naïve group.
   A higher proportion of patients who had stopped IVIg and experienced deterioration during run-in, dropped out of stage A early (approximately 20% within 2 weeks).
   An analysis of patients who received at least 3 efgartigimod injections showed a response rate of 74% to 79% across subgroups.
All secondary endpoints were supportive to the primary endpoint of Stage A, with clinical improvement demonstrated across all parameters (INCAT, I-RODs and GS)

### 1.6.2 Stage B

The primary endpoint was met.
   Efgartigimod significantly reduced the risk of clinical deterioration (relapse) with a hazard ratio of 0.39 (p=0.000039) compared to placebo.
   A hazard ratio of 0.39 equates to a 61% reduction in risk of relapse.
Majority of secondary endpoints and subgroup analyses were supportive.
Additional stage B data:
   At study completion, at the time of 88 events, the proportion of patients with clinical deterioration was 28% in the efgartigimod arm compared to 54% in the placebo arm.
   Fewer relapses compared to placebo during 48-weeks; Kaplan-Meier estimate of relapse in 34% of efgartigimod treated patients compared to 60% treated with placebo.
   Clinical improvement from stage A in INCAT, IRODS and MGS maintained with efgartigimod in stage B, but lost in placebo.

Safety: efgartigimod PH20 SC was well tolerated and had a favourable safety profile in participants with CIDP. No new safety signals were identified.

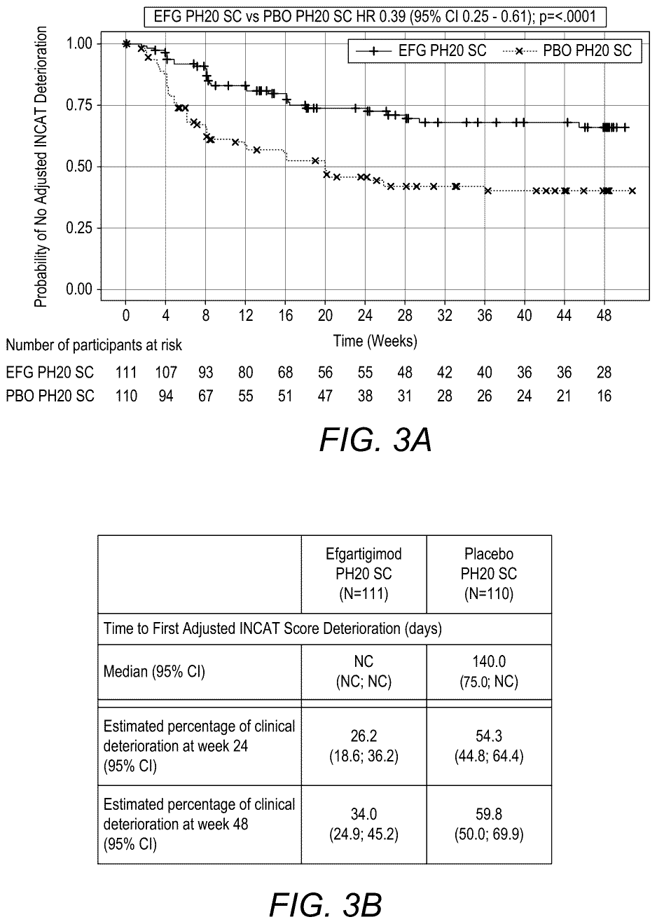

Results from stage B of the trial are presented in FIGS. 3, 4, 5 and 6 and Table 17 below.

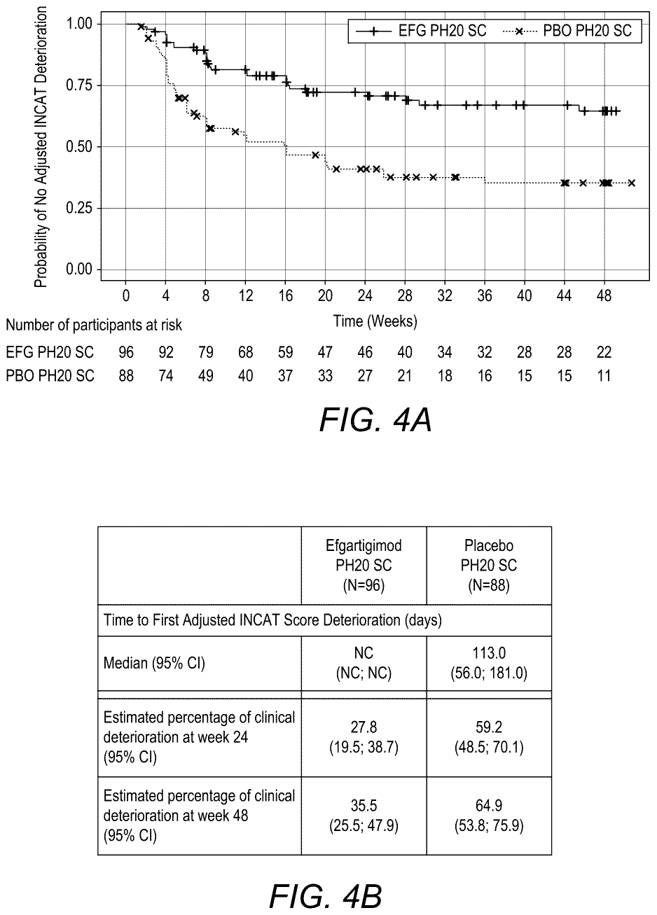

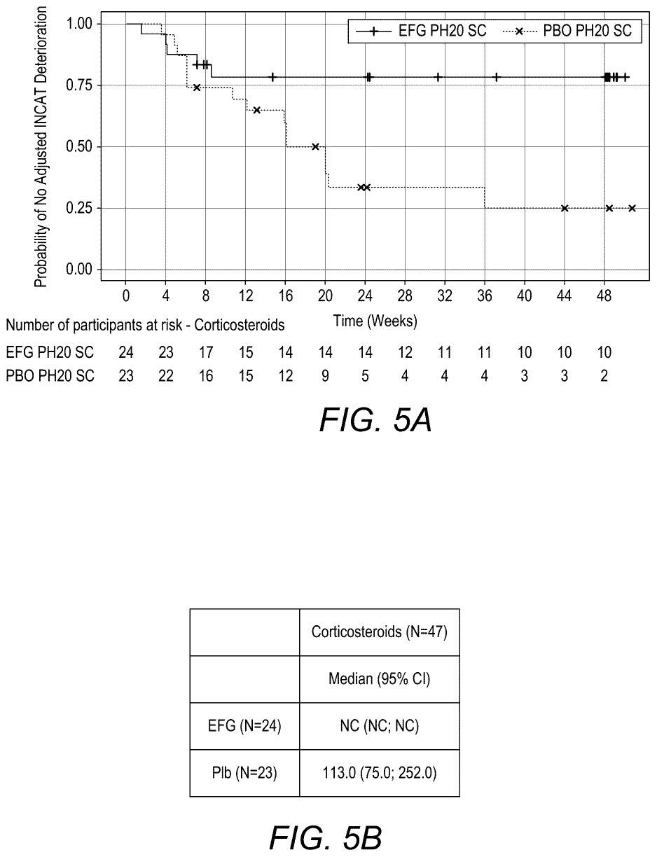

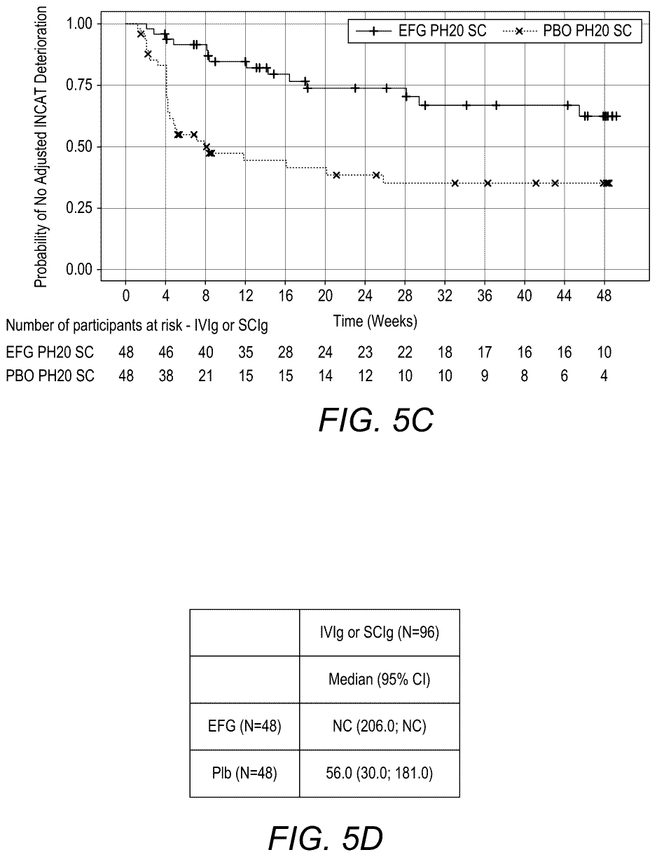

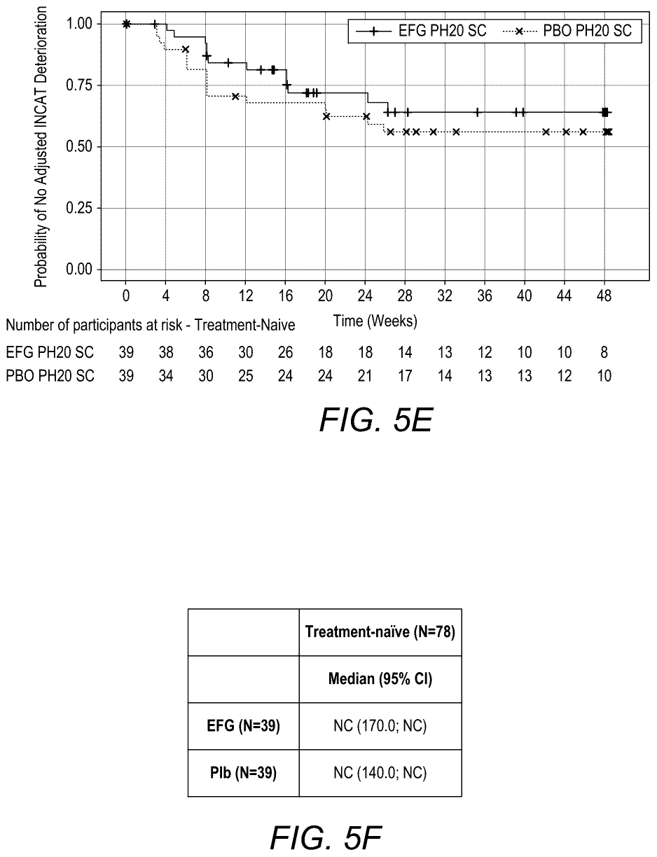

FIGS. 3A-3B, 4A-4B, and 5A-5F show the primary endpoint results for stage B of the trial. FIGS. 3A-3B and 4A-4B show the time to First aINCAT deterioration for patients receiving placebo as compared with patients receiving efgartigimod. FIGS. 5A-5F presents similar results with patients stratified according to prior CIDP medication.

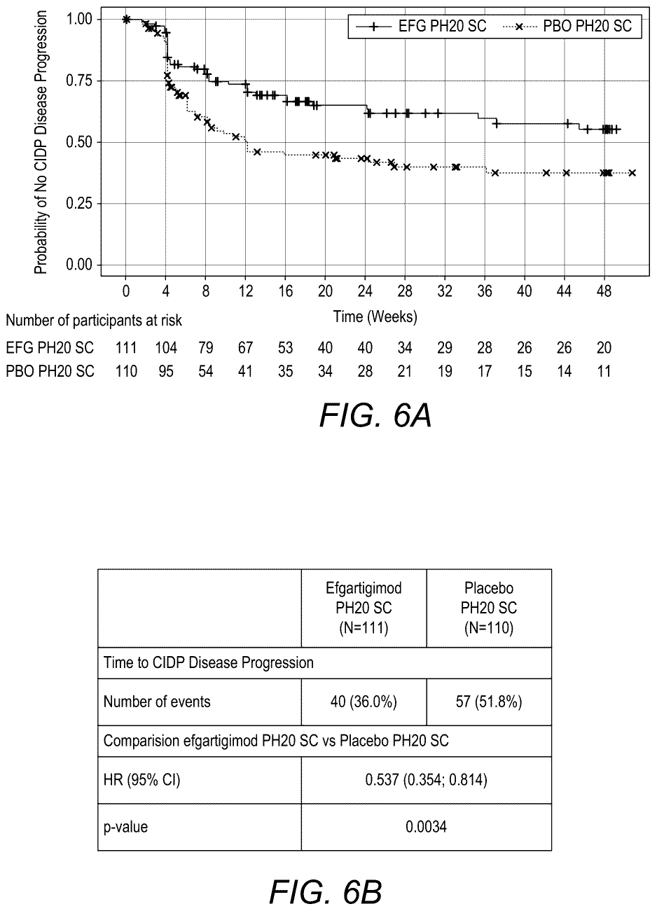

FIGS. 6A-6B shows the results for a stage B secondary endpoint, specifically the time to CIDP disease progression as defined by an I-RODS score decrease of ≥4 points compared to stage B baseline using the centile metric.

Table 17 shows the aINCAT score, I-RODS score and mean grip strength (as measured using both the dominant and non-dominant hands) for patients treated in stage B with either placebo or efgartigimod.

TABLE 17

| | |
|---|---|
| Efgartigimod | Placebo |
| PH20 SC | PH20 SC |
| (N = 111) | (N = 110) |

Mean (SD) change from stage B baseline
to Stage B Last Assessment

| | | |
|---|---|---|
| aINCAT score | 0.1 (1.08) | 0.9 (1.98) |
| | Median = 0.0 | Median = 1.0 |
| I-RODS score | 0.8 (12.33) | −7.0 (19.10) |
| Mean grip | 2.1 (13.29) | −8.2 (20.69) |
| strength - | | |
| dominant hand | | |
| (kPa) | | |
| Mean grip | 2.0 (17.33) | −6.9 (21.30) |
| strength - non- | | |
| dominant hand | | |
| (kPa) | | |

Efgartigimod prevented the level of clinical deterioration seen in the placebo group during stage B of the trial.

The results of this trial are surprising and unexpected for use of efgartigimod in the treatment of CIDP. As described elsewhere herein, CIDP is a heterogenous condition with autoantibodies typically found to be present in only 30-40% of patients. A response rate of 67% is evidence of an efficacy beyond that which could have been expected prior to conduct of this trial. Moreover, the use of IVIg as standard of care in this disease indication was considered by many in the field to be required given the clinical heterogeneity of this disease indication and the multiple modes of action of IVIg (Dalakas et al., 2022). These trial results establish, for the first time, the key involvement of autoantibodies in CIDP pathology and the ability to treat this disease with the use of FcRn antagonists including efgartigimod.

### Example 2: Efficacy and Safety of Efgartigimod PH20 SC in CIDP: ADHERE/ADHERE+ Trials

### Introduction

Efgartigimod, a human immunoglobulin G (IgG)1 antibody Fc fragment, blocks the neonatal Fc receptor, decreasing IgG recycling and reducing pathogenic IgG autoantibody levels. Double-blinded, placebo-controlled ADHERE (NCT04281472), and open-label extension ADHERE+(NCT04280718), assessed the efficacy and safety of efgartigimod PH20 SC (co-formulated with recombinant human hyaluronidase PH20; VYVGART® HYTRULO) in chronic inflammatory demyelinating polyneuropathy (CIDP).

### Methods:

Enrolled participants had CIDP (diagnosis confirmed by external committee) with active disease (treatment-naïve or on standard treatments [withdrawn during a run-in period]). Deteriorating participants received efgartigimod PH20 SC 1000 mg once-weekly (Stage A). Responders were randomized (1:1) to efgartigimod PH20 SC 1000 mg or placebo once-weekly (Stage B). Participants with clinical deterioration in Stage B or who completed ADHERE could enter ongoing ADHERE+(efgartigimod PH20 SC 1000 mg QW). Primary outcomes were ECI (assessed with aINCAT, I-RODS, or mean grip strength; Stage A), efficacy (time to first aINCAT score deterioration [relapse]; Stage B), and safety (treatment-emergent adverse events [TEAEs]; ADHERE+).

### Results:

The efficacy of VYVGART HYTRULO for the treatment of adults with CIDP was demonstrated in a prospective, multicenter study conducted in 2 treatment stages: an open-label stage A and a randomized-withdrawal, double-blinded, placebo-controlled stage B (NCT04281472).

Data from Stage A and Stage B of ADHERE are described in Example 1. Briefly, a total of 322 patients enrolled in stage A, including both non-treatment-naïve and treatment-naïve patients, received up to 12 once weekly injections of VYVGART HYTRULO 1008 mg/11,200 units until ECI occurred at 2 consecutive study visits. A total of 221 patients were randomized in stage B with 111 receiving VYVGART HYTRULO 1008 mg/11,200 units and 110 receiving placebo once weekly.

Baseline characteristics of stage B were similar between treatment groups. Patients had a median age of 55 years (range: 20 to 82 years), a median time since CIDP diagnosis of 2.2 years and median INCAT score of 3.0. Sixty-four percent were male and 65% were White.

Patients were stratified based on the most recent CIDP treatment within 6 months before study entry: 35.3% of patients were treatment-naïve, 21.3% of patients had received corticosteroids and 43.4% of patients had received Immunoglobulins.

In stage A, the primary endpoint was the percentage of responders with the time to the first ECI as secondary endpoint. The results are presented in Table 18. In Stage A, 214/322 (66.5%) participants demonstrated ECI.

TABLE 18
ECI in Patients with CIDP in ADHERE Stage A - ECI
Responders and Time to Initial Confirmed ECI

| | Stage A |
|---|---|
| | VYVGART |
| | HYTRULO |
| | (N = 322) |
| ECI Responders (patients with confirmed clinical | 214/322 (66.5%) |
| improvement) | (61.0; 71.6) |
| n/N (%) (95% CI) | |
| Time to initial confirmed ECI in days | 43.0 |
| median (95% CI) | (31.0; 51.0) |

ECI: evidence of clinical improvement defined as clinical improvement on the parameters that the patient worsened in the period between screening and stage A (I-RODS, Grip Strength) or clinical improvement in INCAT;
n = number of patients for whom the observation was reported; N = number of patients in the analysis set; %: percentage; aINCAT: adjusted Inflammatory Neuropathy Cause and Treatment.

In stage B, the primary endpoint was defined as the time to the occurrence of the first evidence of clinical deterioration (≥1-point increase in aINCAT compared to stage B baseline, which was confirmed at a consecutive visit after the first 1-point increase in aINCAT or not confirmed for patients with ≥2-point increase in aINCAT compared to stage B baseline): Patients who received VYVGART HYTRULO remained relapse-free (i.e., no clinical deterioration) significantly longer compared to patients who received placebo, as demonstrated by a hazard ratio of 0.394 [95% CI (0.253; 0.614) p=0.000039] which represents a 61% risk reduction for deterioration in patients treated with VYVGART HYTRULO. The results are presented in Table 19 and FIG. 3A.

At week 4, the earliest time point for which ECI criteria could have been met, up to 40% of patients had ECI. Based on additional pre-specified analysis, 25% of patients showed clinically relevant improvement after 9 days in at least one of the 3 parameters (aINCAT, I-RODS or Grip Strength).

Across all prior CIDP medication subgroups, the majority of patients had a response to treatment with VYVGART HYTRULO.

TABLE 19
First Evidence of Clinical Deterioration in Patients with
CIDP in ADHERE Stage B -Time to 1^{st} aINCAT Increase

| Stage B | |
|---|---|
| VYVGART | |
| HYTRULO | Placebo |
| (N = 111) | (N = 110) |

Time to 1^{st} aINCAT increase
(clinical deterioration) in days

| Hazard ratio (95% CI) | 0.394 (0.253; 0.614) | |
|---|---|---|
| | p-value 0.000039 | |
| Median time (95% CI) | NC (NC; NC) | 140.0 (75.0; NC) |

NC: not calculated; n = number of patients for whom the observation was reported; N = number of patients in the analysis set; %: percentage; aINCAT: adjusted Inflammatory Neuropathy Cause and Treatment.

99% of eligible participants entered ADHERE+. No new safety signals emerged in ADHERE+; 57.5% of participants had ≥1 TEAE (most mild/moderate) and 9.2% had ≥1 serious adverse event (one treatment-related death occurred). Participants who relapsed in Stage B (efgartigimod: 27.9%; PBO: 53.6%) demonstrated clinical improvement in aINCAT score in ADHERE+; those who did not relapse maintained their aINCAT scores in ADHERE+. Clinical improvements were also demonstrated with I-RODS and mean grip strength.

### Conclusions

ADHERE+ demonstrated long-term effectiveness of efgartigimod PH20 for prevention of relapse. Efgartigimod PH20's safety profile was similar between ADHERE and ADHERE+ and longer exposure did not lead to increased TEAE frequency or severity.

### Example 3: Label with Prescribing Information for VYVGART HYTRULO

The text below is from the label with the prescribing information for VYVGART® HYTRULO (efgartigimod alfa and hyaluronidase-qvfc) injection, for subcutaneous use, as approved by Food and Drug Administration (Initial U.S. Approval: 2023, revised xx/xxxx).

### Highlights of Prescribing Information

These highlights do not include all the information needed to use VYVGART HYTRULO safely and effectively. See full prescribing information for VYVGART HYTRULO. VYVGART® HYTRULO (efgartigimod alfa and hyaluronidase-qvfc) injection, for subcutaneous use Initial U.S. Approval: 2023

### Recent Major Changes

Indications and Usage (1) x/202x
Dosage and Administration (2.4) x/202x
Contraindications (4) 12/2023
Warnings and Precautions (5.2, 5.3) 12/2023

### Indications and Usage

VYVGART HYTRULO is a combination of efgartigimod alfa, a neonatal Fc receptor blocker, and hyaluronidase, an endoglycosidase, indicated for the treatment of adult patients with:
generalized myasthenia gravis (gMG) who are anti-acetylcholine receptor (AChR) antibody positive. (1)
chronic inflammatory demyelinating polyneuropathy (CIDP) (1) DOSAGE AND ADMINISTRATION

See Full Prescribing Information for instructions on dosage, preparation, and administration. (2.1, 2.2, 2.3, 2.4, 2.5)

Evaluate the need to administer age-appropriate vaccines according to immunization guidelines before initiation of a new treatment cycle with VYVGART HYTRULO. (2.1)

Administer by a healthcare professional only. (2.2)

Administer with a winged infusion set subcutaneously over 30 to 90 seconds. (2.2, 2.3, 2.4)

gMG: The recommended dosage is 1,008 mg/11,200 units (1,008 mg efgartigimod alfa and 11,200 units hyaluronidase) in cycles of once weekly injections for 4 weeks. Administer subsequent treatment cycles based on clinical evaluation; safety of initiating subsequent cycles sooner than 50 days from the start of the previous treatment cycle has not been established. (2.3)

CIDP: The recommended dosage is 1,008 mg/11,200 units (1,008 mg efgartigimod alfa and 11,200 units hyaluronidase) as once weekly. (2.4)

### Dosage Forms and Strengths

Injection: 1,008 mg efgartigimod alfa and 11,200 units hyaluronidase per 5.6 mL (180 mg/2,000 units per mL) in a single-dose vial. (3)

### Contraindications

VYVGART HYTRULO is contraindicated in patients with serious hypersensitivity to efgartigimod alfa products, to hyaluronidase, or to any of the excipients of VYVGART HYTRULO. (4)

### Warnings and Precautions

Infections: Delay administration of VYVGART HYTRULO to patients with an active infection. Monitor for signs and symptoms of infection in patients treated with VYVGART HYTRULO. If serious infection occurs, administer appropriate treatment and consider withholding VYVGART HYTRULO until the infection has resolved. (5.1)

Hypersensitivity Reactions: Anaphylaxis, hypotension leading to syncope, angioedema, dyspnea, rash, and urticaria have occurred in patients treated with VYVGART HYTRULO or intravenous efgartigimod alfa-fcab product. If a hypersensitivity reaction occurs, the healthcare professional should institute appropriate measures if needed or the patient should seek medical attention. (4, 5.2)

Infusion-Related Reactions: If a severe infusion-related reaction occurs, initiate appropriate therapy; consider the risks and benefits of readministering. If a mild to moderate infusion-related reaction occurs, may rechallenge with close clinical observation, slower infusion rates, and pre-medications. (5.3)

### Adverse Reactions

The most common adverse reactions (≥10%) in patients with gMG treated with efgartigimod alfa-fcab were respiratory tract infections, headache, and urinary tract infection.

Injection site reactions were common (≥15%) in patients with gMG and CIDP who were treated with VYVGART HYTRULO. (6.1)

To report SUSPECTED ADVERSE REACTIONS, contact Argenx at 1-833-argx411 or FDA at 1-800-FDA-1088 or fda.gov/medwatch.

### Drug Interactions

Closely monitor for reduced effectiveness of medications that bind to the human neonatal Fc receptor. When concomitant long-term use of such medications is essential for patient care, consider discontinuing VYVGART HYTRULO and using alternative therapies. (7)

See 17 for PATIENT COUNSELING INFORMATION

### Full Prescribing Information: Contents*

1 INDICATIONS AND USAGE
2 DOSAGE AND ADMINISTRATION
2.1 Recommended Vaccination
2.2 Important Dosage and Administration Instructions
2.3 Recommended Dosage for gMG
2.4 Recommended Dosage for CIDP
2.5 Preparation and Administration Instructions
3 DOSAGE FORMS AND STRENGTHS
4 CONTRAINDICATIONS
5 WARNINGS AND PRECAUTIONS
5.1 Infections
5.2 Hypersensitivity Reactions
5.3 Infusion-Related Reactions
6 ADVERSE REACTIONS
6.1 Clinical Trials Experience
6.2 Postmarketing Experience
7 DRUG INTERACTIONS
7.1 Effect of VYVGART HYTRULO on Other Drugs
8 USE IN SPECIFIC POPULATIONS
8.1 Pregnancy
8.4 LactationPediatric Use
8.5 Geriatric Use
8.6 Renal Impairment
11 DESCRIPTION
12 CLINICAL PHARMACOLOGY
12.1 Mechanism of Action
12.2 Pharmacodynamics
12.3 Pharmacokinetics
12.6 Immunogenicity
13 NONCLINICAL TOXICOLOGY
13.1 Carcinogenesis, Mutagenesis, Impairment of Fertility
14 CLINICAL STUDIES
14.1 Generalized Myasthenia Gravis
14.2 Chronic Inflammatory Demyelinating Polyneuropathy
16 HOW SUPPLIED/STORAGE AND HANDLING
17 PATIENT COUNSELING INFORMATION
   Sections or subsections omitted from the full prescribing information are not listed

### 1 Indications and Usage

VYVGART HYTRULO is indicated for the treatment of adult patients with:
generalized myasthenia gravis (gMG) who are anti-acetylcholine receptor (AChR) antibody positive.
chronic inflammatory demyelinating polyneuropathy (CIDP)

### 2 Dosage and Administration

### 2.1 Recommended Vaccination

Because VYVGART HYTRULO causes transient reduction in IgG levels, immunization with live-attenuated or live vaccines is not recommended during treatment with VYVGART HYTRULO. Evaluate the need to administer age-appropriate immunizations according to immunization guidelines before initiation of a new treatment cycle with VYVGART HYTRULO [see Dosage and Administration (2.3) and Warnings and Precautions (5.1)].

### 2.2 Important Dosage and Administration Instructions

VYVGART HYTRULO is to be administered by a healthcare professional only.

VYVGART HYTRULO is for subcutaneous use only with a winged infusion set [see Dosage and Administration (2.4)]. Do not administer intravenously.

Do not dilute VYVGART HYTRULO.

### 2.3 Recommended Dosage for gMG

The recommended dosage of VYVGART HYTRULO is 1,008 mg/11,200 units (1,008 mg efgartigimod alfa and 11,200 units hyaluronidase) administered subcutaneously over approximately 30 to 90 seconds in cycles of once weekly injections for 4 weeks.

Administer subsequent treatment cycles according to clinical evaluation. The safety of initiating subsequent cycles sooner than 50 days from the start of the previous treatment cycle has not been established.

If a scheduled dose is missed, VYVGART HYTRULO may be administered up to 3 days after the scheduled time point. Thereafter, resume the original dosing schedule until the treatment cycle is completed.

### 2.4 Recommended Dosage for CIDP

The recommended dosage of VYVGART HYTRULO is 1,008 mg/11,200 units (1,008 mg efgartigimod alfa and 11,200 units hyaluronidase) administered subcutaneously over approximately 30 to 90 seconds as once weekly injections.

If a scheduled injection is missed, VYVGART HYTRULO may be administered up to 3 days after the scheduled time point. Thereafter, resume the original dosing schedule.

### 2.5 Preparation and Administration Instructions

Use aseptic technique when preparing and administering VYVGART HYTRULO. Do not shake the vial. Each vial is for one time use only. Avoid exposure to direct sunlight.

### Preparation

Take the VYVGART HYTRULO vial out of the refrigerator at least 15 minutes before injecting to allow it to reach room temperature [see How Supplied/Storage and Handling (16)]. Do not use external heat sources.
Check that the VYVGART HYTRULO solution is yellowish, clear to opalescent.
Parenteral medicine products should be inspected visually for particulate matter prior to administration, whenever solution and container permit. Do not use if opaque particles or other foreign particles are present.
Withdraw the entire content of VYVGART HYTRULO from the vial using a polypropylene syringe and an 18 G stainless steel transfer needle.
Remove large air bubbles, if present.
Each vial contains overfill to compensate for liquid loss during preparation and to compensate for the priming volume of the winged infusion set.
VYVGART HYTRULO vial does not contain preservatives. Administer immediately after preparation.

### Administration

To administer VYVGART HYTRULO vial, use a winged infusion set made of polyvinyl chloride (PVC), 25 G, 12 inches tubing, maximum priming volume of 0.4 mL.
Remove the transfer needle from the syringe and connect the syringe to the winged infusion set.
Prior to administration, fill the tubing of the winged infusion set by gently pressing the syringe plunger until the plunger is at 5.6 mL. There should be solution at the end of the winged infusion set needle.
Choose an injection site on the abdomen (at least 2 to 3 inches away from the navel).
Do not inject on areas where the skin is red, bruised, tender, hard, or into areas where there are moles or scars.
Rotate injection sites for subsequent administrations.
Inject VYVGART HYTRULO vial subcutaneously into a pinched skin area at an angle of about 45 degrees over 30 to 90 seconds.
Localized injection site reactions may occur after VYVGART HYTRULO is administered. [see Adverse Reactions (6.1)].
Discard any unused portions of medicine remaining in the vial, the syringe and the winged infusion set.
Healthcare professionals should monitor for clinical signs and symptoms of hypersensitivity reactions for at least 30 minutes after administration. If a hypersensitivity reaction occurs, the healthcare professional should institute appropriate measures if needed or the patient should seek medical attention [see Warnings and Precautions (5.2)].

### 3 Dosage Forms and Strengths

Injection: 1,008 mg efgartigimod alfa and 11,200 units hyaluronidase per 5.6 mL (180 mg/2,000 units per mL) as yellowish, clear to opalescent solution, in a single-dose vial.

### 4 Contraindications

VYVGART HYTRULO is contraindicated in patients with serious hypersensitivity to efgartigimod alfa products, to hyaluronidase, or to any of the excipients of VYVGART HYTRULO. Reactions have included anaphylaxis and hypotension leading to syncope [see Warnings and Precautions (5.2)].

### 5 Warnings and Precautions

### 5.1 Infections

VYVGART HYTRULO may increase the risk of infection. The most common infections observed in Study 1 were urinary tract infection (10% of efgartigimod alfa-fcab-treated patients compared to 5% of placebo-treated patients) and respiratory tract infections (33% of efgartigimod alfa-fcab-treated patients compared to 29% of placebo-treated patients) [see Adverse Reactions (6.1) and Clinical Studies (14)]. A higher frequency of patients who received efgartigimod alfa-fcab compared to placebo were observed to have below normal levels for white blood cell counts (12% versus 5%, respectively), lymphocyte counts (28% versus 19%, respectively), and neutrophil counts (13% versus 6%, respectively). The majority of infections and hematologic abnormalities were mild to moderate in severity. Delay VYVGART HYTRULO administration in patients with an active infection until the infection is resolved. During treatment with VYVGART HYTRULO, monitor for clinical signs and symptoms of infections. If serious infection occurs, administer appropriate treatment and consider withholding VYVGART HYTRULO until the infection has resolved.

### Immunization

Immunization with vaccines during VYVGART HYTRULO treatment has not been studied. The safety of immunization with live or live-attenuated vaccines and the response to immunization with any vaccine are unknown. Because VYVGART HYTRULO causes a reduction in IgG levels, vaccination with live-attenuated or live vaccines is not recommended during treatment with VYVGART HYTRULO. Evaluate the need to administer age-appropriate vaccines according to immunization guidelines before initiation of a new treatment cycle with VYVGART HYTRULO.

### 5.2 Hypersensitivity Reactions

In clinical trials, hypersensitivity reactions, including rash, angioedema, and dyspnea were observed in patients treated with VYVGART HYTRULO or intravenous efgartigimod alfa-fcab. Urticaria was also observed in patients treated with VYVGART HYTRULO. Hypersensitivity reactions were mild or moderate, occurred within one hour to three weeks of administration.

Anaphylaxis and hypotension leading to syncope have been reported in postmarketing experience with intravenous efgartigimod alfa-fcab. Anaphylaxis and hypotension occurred during or within an hour of administration and led to infusion discontinuation and in some cases to permanent treatment discontinuation.

Healthcare professionals should monitor for clinical signs and symptoms of hypersensitivity reactions for at least 30 minutes after administration [see Dosage and Administration (2.4)]. If a hypersensitivity reaction occurs, the healthcare professional should institute appropriate measures if needed or the patient should seek medical attention. VYVGART HYTRULO is contraindicated in patients with a history of serious hypersensitivity to efgartigimod alfa products, to hyaluronidase, or to any of the excipients of VYVGART HYTRULO [see Contraindications (4)].

### 5.3 Infusion-Related Reactions

Infusion-related reactions have been reported with intravenous efgartigimod alfa-fcab in postmarketing experience. The most frequent symptoms and signs were hypertension, chills, shivering, and thoracic, abdominal, and back pain. Infusion-related reactions occurred during or within an hour of administration and led to infusion discontinuation. If a severe infusion-related reaction occurs, initiate appropriate therapy. Consider the risks and benefits of readministering VYVGART HYTRULO following a severe infusion-related reaction. If a mild to moderate infusion-related reaction occurs, patients may be rechallenged with close clinical observation, slower infusion/injection rates, and pre-medications.

### 6 Adverse Reactions

The following clinically significant adverse reactions are described elsewhere in the labeling:
Infections [see Warnings and Precautions (5.1)]
Hypersensitivity Reactions [see Warnings and Precautions (5.2)]
Infusion-Related Reactions [see Warnings and Precautions (5.3)]

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

Clinical Experience in Patients with gMG

The safety of efgartigimod alfa in patients with gMG was established in a double blinded placebo-controlled study with efgartigimod alfa-fcab administered intravenously (Study 1) and its open-label extension, and in an active-controlled study of VYVGART HYTRULO administered subcutaneously (Study 2) and its open-label extension [see Clinical Studies (14.1)].

Adverse Reactions with Efgartigimod Alfa-fcab Intravenous in Patients with gMG

In clinical studies, the safety of efgartigimod alfa-fcab administered intravenously has been evaluated in 246 patients with gMG who received at least one dose of efgartigimod alfa-fcab, including 57 patients exposed to at least 7 treatment cycles and 8 patients exposed to at least 10 treatment cycles.

In a placebo-controlled study (Study 1) in patients with gMG, 84 patients received efgartigimod alfa-fcab 10 mg/kg [see Clinical Studies (14)]. Of these 84 patients, approximately 75% were female, 82% were White, 11% were Asian, and 8% were of Hispanic or Latino ethnicity. The mean age at study entry was 46 years (range 19 to 78).

The minimum time to initiate a subsequent cycle, specified by study protocol, was 50 days from the start of the previous treatment cycle. On average, efgartigimod alfa-fcab-treated patients received 2 cycles in Study 1. The mean and median times to the second treatment cycle were 94 days and 72 days from the initial infusion of the first treatment cycle, respectively, for efgartigimod alfa-fcab-treated patients.

Adverse reactions reported in at least 5% of patients treated with efgartigimod alfa-fcab and more frequently than placebo are summarized in Table 20. The most common adverse reactions (reported in at least 10% of efgartigimod alfa-fcab-treated patients) were respiratory tract infection, headache, and urinary tract infection.

TABLE 20
Adverse Reactions in at least 5% of Patients with gMG Treated with
Efgartigimod Alfa-fcab Intravenously (EFG IV) and More Frequently
than in Placebo-Treated Patients in Study 1 (Safety Population)

| | EFG IV | Placebo |
|---|---|---|
| | (N = 84) | (N = 83) |
| Adverse reaction | % | % |
| Respiratory tract infection | 33 | 29 |
| Headache* | 32 | 29 |
| Urinary tract infection | 10 | 5 |
| Paraesthesia^{†} | 7 | 5 |
| Myalgia | 6 | 1 |

*Headache includes migraine and procedural headache.
^{†}Paresthesia includes oral hypoesthesia, hypoesthesia, and hyperesthesia.

Adverse Reactions with VYVGART HYTRULO in Patients with gMG

In an active-controlled study in patients with gMG (Study 2), 110 patients were randomized and received one cycle of once weekly administrations for 4 weeks (4 administrations total), of either VYVGART HYTRULO subcutaneously (n=55) or efgartigimod alfa-fcab intravenously (n=55) at recommended doses [see Dosage and Administration (2.2)]. The open-label extension of Study 2 included some patients who switched from efgartigimod alfa-fcab IV to VYVGART HYTRULO.

The most common adverse reactions (reported in at least 10% of VYVGART HYTRULO-treated patients) were injection site reactions and headache.

In Study 2, injection site reactions occurred in 38% of patients receiving VYVGART HYTRULO. These were injection site rash, erythema, pruritus, bruising, pain, and urticaria.

In Study 2 and its open-label extension (n=168), all injection site reactions were mild to moderate in severity and did not lead to treatment discontinuation. The majority occurred within 24 hours after administration and resolved spontaneously. Most injection site reactions occurred during the first treatment cycle, and the incidence decreased with each subsequent cycle.

Clinical Experience in Patients with CIDP
Adverse Reactions with VYVGART HYTRULO in Patients with CIDP

In a placebo-controlled study in patients with CIDP (Study 3, stage B), 221 patients were randomized to receive once-weekly administration of either VYVGART HYTRULO 1,008 mg/11, 200 units subcutaneously (n=111) or placebo (n=110) [see Clinical Studies (14.2)]. The mean duration of treatment with VYVGART HYTRULO in stage B was 25 weeks. The overall safety profile observed in patients with CIDP treated with VYVGART HYTRULO was consistent with the known safety profile of VYVGART HYTRULO and of efgartigimod alfa-fcab administered intravenously.

In Study 3, injection site reactions occurred in 15% of patients treated with VYVGART HYTRULO compared to 6% of patients who received placebo. The most common of these injection site reactions were injection site bruising and injection site erythema. All injection site reactions were mild to moderate in severity. Most injection site reactions occurred during the first 3 months of treatment.

### 6.2 Postmarketing Experience

The following adverse reactions have been identified during postapproval use of efgartigimod alfa products. Because these reactions are reported voluntarily from a population of uncertain size, it is not always possible to reliably estimate their frequency or establish a causal relationship to drug exposure.

Immune System Disorders: Hypersensitivity reactions including anaphylaxis and hypotension, and infusion-related reactions [see Warnings and Precautions (5.2, 5.3)].

### 7 Drug Interactions

### 7.1 Effect of VYVGART HYTRULO on Other Drugs

Concomitant use of VYVGART HYTRULO with medications that bind to the human neonatal Fc receptor (FcRn) (e.g., immunoglobulin products, monoclonal antibodies, or antibody derivates containing the human Fc domain of the IgG subclass) may lower systemic exposures and reduce effectiveness of such medications. Closely monitor for reduced effectiveness of medications that bind to the human neonatal Fc receptor. When concomitant long-term use of such medications is essential for patient care, consider discontinuing VYVGART HYTRULO and using alternative therapies.

### 8 Use in Specific Populations

### 8.1 Pregnancy

### Pregnancy Exposure Registry

There is a pregnancy exposure registry that monitors pregnancy outcomes in women exposed to VYVGART HYTRULO during pregnancy. Healthcare providers and patients may call 1-855-272-6524 or go to https://www.Vyvgartpregnancy.com to enroll in or to obtain information about the registry.

### Risk Summary

There are no available data on the use of VYVGART HYTRULO or efgartigimod alfa containing products during pregnancy. There was no evidence of adverse developmental outcomes following the intravenous administration of efgartigimod alfa at up to 100 mg/kg/day in rats and rabbits (see Data).

The background rate of major birth defects and miscarriage in the indicated population is unknown. In the U.S. general population, the estimated background rate of major birth defects and miscarriage in clinically recognized pregnancies is 2% to 4% and 15% to 20%, respectively.

### Clinical Considerations

### Fetal/Neonatal Adverse Reactions

Monoclonal antibodies are increasingly transported across the placenta as pregnancy progresses, with the largest amount transferred during the third trimester. Therefore, efgartigimod alfa may be transmitted from the mother to the developing fetus.

As VYVGART HYTRULO is expected to reduce maternal IgG antibody levels, reduction in passive protection to the newborn is anticipated. Risk and benefits should be considered prior to administering live or live-attenuated vaccines to infants exposed to VYVGART HYTRULO in utero [see Warnings and Precautions (5.1)].

### Data

### Animal Data

VYVGART HYTRULO for subcutaneous injection contains efgartigimod alfa and hyaluronidase [see Description (11)].

### Efgartigimod Alfa:

Intravenous administration of efgartigimod alfa (0, 30, or 100 mg/kg/day) to pregnant rats and rabbits throughout organogenesis resulted in no adverse effects on embryofetal development in either species. Maternal efgartigimod alfa exposures at the highest no-effect doses were approximately 8 and 62 times, respectively, that in humans at the recommended human dose (RHD) of 1008 mg.
Intravenous administration of efgartigimod alfa (0, 30, or 100 mg/kg/day) to rats throughout gestation and lactation resulted in no adverse effects on pre- or postnatal development. Maternal exposures at the highest no-effect dose were approximately 13 times that in humans at the RHD.

### Hyaluronidase:

In a study in which hyaluronidase (human recombinant) was administered by subcutaneous injection to pregnant mice throughout organogenesis, increased embryofetal mortality and decreased fetal body weights were observed at the highest doses tested. The no-effect dose for adverse effects on embryofetal development in the mouse was approximately 1800 times the dose of hyaluronidase at the recommended human dose (RHD) of VYVGART HYTRULO (1,008 mg efgartigimod alfa and 11,200 U hyaluronidase), on a U/kg basis.
There were no adverse effects on pre- and postnatal development following subcutaneous administration of hyaluronidase (human recombinant) to mice throughout gestation and lactation at doses up to 5,000 times the dose of hyaluronidase at the RHD of VYVGART HYTRULO, on a U/kg basis.

### 8.2 Lactation

### Risk Summary

There is no information regarding the presence of efgartigimod alfa or hyaluronidase, from administration of VYVGART HYTRULO, in human milk, the effects on the breastfed infant, or the effects on milk production. Maternal IgG is known to be present in human milk.

The developmental and health benefits of breastfeeding should be considered along with the mother's clinical need for VYVGART HYTRULO and any potential adverse effects on the breastfed infant from VYVGART HYTRULO or from the underlying maternal condition.

### 8.4 Pediatric Use

Safety and effectiveness in pediatric patients have not been established.

### 8.5 Geriatric Use

Clinical studies of VYVGART HYTRULO did not include sufficient numbers of patients aged 65 and older to determine whether they respond differently from younger adult patients.

### 8.6 Renal Impairment

No dose adjustment of VYVGART HYTRULO is needed for patients with mild renal impairment. There are insufficient data to evaluate the impact of moderate renal impairment (eGFR 30-59 mL/min/1.73 m²) and severe renal impairment (eGFR <30 mL/min/1.73 m²) on pharmacokinetic parameters of VYVGART HYTRULO [see Clinical Pharmacology (12.3)].

### 11 Description

VYVGART HYTRULO is a coformulation of efgartigimod alfa and hyaluronidase (human recombinant).

Efgartigimod alfa, a neonatal Fc receptor blocker, is a human immunoglobulin G1 (IgG1)-derived Fc fragment (fragment, crystallized) of the za allotype, produced in Chinese hamster ovary (CHO) cells. The efgartigimod alfa Fc fragment is a homodimer consisting of two identical peptide chains each consisting of 227 amino acids linked together by two interchain disulfide bonds with affinity for FcRn. The molecular weight of efgartigimod alfa is approximately 54 kDa.

Hyaluronidase (human recombinant) is an endoglycosidase used to increase the dispersion and absorption of co-administered drugs when administered subcutaneously. Hyaluronidase (human recombinant) is a glycosylated single-chain protein produced by Chinese hamster ovary cells containing a DNA plasmid encoding for a soluble fragment of human hyaluronidase (PH20). Hyaluronidase (human recombinant) has a molecular weight of approximately 61 kDa.

VYVGART HYTRULO (efgartigimod alfa and hyaluronidase-qvfc) injection is a sterile, preservative free, yellowish, clear to opalescent solution supplied in a single-dose vial for subcutaneous injection.

Each 5.6 mL single-dose vial of VYVGART HYTRULO contains 1,008 mg efgartigimod alfa and 11,200 units hyaluronidase (human recombinant). Each mL of solution contains 180 mg of efgartigimod alfa, 2,000 units of hyaluronidase (human recombinant) and histidine (1.4 mg), L-histidine hydrochloride monohydrate (2.2 mg), methionine (1.5 mg), polysorbate 20 (0.4 mg), sodium chloride (5.8 mg), sucrose (20.5 mg), and water for injection, USP, at a pH of 6.0.

### 12 Clinical Pharmacology

### 12.1 Mechanism of Action

VYVGART HYTRULO is a coformulation of efgartigimod alfa and hyaluronidase.

Efgartigimod alfa is a human IgG1 antibody fragment that binds to the neonatal Fc receptor (FcRn), resulting in the reduction of circulating IgG.

Hyaluronidase increases permeability of the subcutaneous tissue by depolymerizing hyaluronan. This effect is transient and permeability of the subcutaneous tissue is restored within 24 to 48 hours.

### 12.2 Pharmacodynamics

In Study 1 [see Clinical Studies (14)], the pharmacological effect of efgartigimod alfa-fcab was assessed by measuring the decrease in serum IgG levels and AChR autoantibody levels. In patients testing positive for AChR antibodies and who were treated with efgartigimod alfa-fcab intravenous, there was a reduction in total IgG levels relative to baseline. Decrease in AChR autoantibody levels followed a similar pattern. A decrease in AChR-Ab was associated with a clinical response in AChR-Ab positive patients, as measured by the change from baseline in MG-ADL total score.

In Study 2, the pharmacological effect of VYVGART HYTRULO administered subcutaneously (SC) at 1,008 mg/11,200 Units was compared to efgartigimod alfa-fcab administered intravenously at 10 mg/kg (EFG IV) in gMG patients. The maximum mean reduction in AChR-Ab level was observed at week 4, with a mean reduction of 62.2% and 59.7% in the VYVGART HYTRULO SC and efgartigimod alfa-fcab IV arm, respectively. The decrease in total IgG levels followed a similar pattern. The 90% confidence intervals for the geometric mean ratios of AChR-Ab reduction at day 29 and AUEC_{0-4 w} (area under the effect-time curve from time 0 to 4 weeks post dose) were within the range of 80% to 125%, indicating no clinically significant difference between the two formulations.

### 12.3 Pharmacokinetics

Efgartigimod alfa exposures were approximately dose-proportional up to the highest subcutaneously tested dose of VYVGART HYTRULO (1750 mg, 1.75 times the recommended dosage).

### Distribution

The volume of distribution is 15 to 20L.

### Metabolism and Elimination

Efgartigimod alfa and hyaluronidase are expected to be degraded by proteolytic enzymes into small peptides and amino acids.

The terminal half-life is 80 to 120 hours (3 to 5 days).

After a single intravenous dose of 10 mg/kg efgartigimod alfa-fcab in healthy subjects, less than 0.1% of the administered dose was recovered in urine.

### Specific Populations

### Age, Sex and Race

A population pharmacokinetics analysis assessing the effects of age, body weight, sex, and race did not suggest any clinically significant impact of these covariates on efgartigimod alfa exposures.

### Body Weight

A population pharmacokinetics analysis suggests that the influence of body weight on efgartigimod alfa exposure after administration of VYVGART HYTRULO SC 1008 mg was limited and not clinically relevant.

Patients with Renal Impairment

No dedicated pharmacokinetic study has been performed in patients with renal impairment.

Population PK analyses of data from the VYVGART HYTRULO clinical studies indicated that patients with mild renal impairment (eGFR 60-89 mL/min/1.73 m²) had 11 to 20% increase in exposure relative to the exposure in patients with normal renal function [see Use in Specific Populations (8.6)].

Patients with Hepatic Impairment

No dedicated pharmacokinetic study has been performed in patients with hepatic impairment. Hepatic impairment is not expected to affect the pharmacokinetics of efgartigimod alfa.

### Drug Interaction Studies

Clinical drug interactions studies have not been performed with efgartigimod alfa.

### P450 Enzymes

Efgartigimod alfa is not metabolized by cytochrome P450 enzymes; therefore, interactions with concomitant medications that are substrates, inducers, or inhibitors of cytochrome P450 enzymes are unlikely.

Drug Interactions with Other Drugs or Biological Products

Efgartigimod alfa may decrease concentrations of compounds that bind to the human FcRn [see Drug Interactions (7.1)].

### 12.6 Immunogenicity

The observed incidence of anti-drug antibodies is highly dependent on the sensitivity and specificity of the assay. Differences in assay methods preclude meaningful comparisons of the incidence of anti-drug antibodies in the studies described below with the incidence of anti-drug antibodies in other studies, including those of VYVGART HYTRULO or of other efgartigimod products.

In Study 2, in up to 10 weeks following the initiation of a treatment period with 4 weekly administrations, the incidence of anti-efgartigimod alfa antibodies was 35% (19/55) following treatment with VYVGART HYTRULO and 20% (11/55) in patients receiving intravenous efgartigimod alfa-fcab. For both IV and SC arms, neutralizing anti-efgartigimod alfa antibodies were detected in 4% (2/55) of patients.

In Study 3, in up to 12 weeks of treatment in stage A and 48 weeks in stage B, the incidence of anti-efgartigimod alfa antibodies was 6% (20/317) in stage A and 2% (2/111) in stage B, following treatment with VYVGART HYTRULO. Neutralizing anti-efgartigimod alfa antibodies were detected in 0.3% (1/317) of patients in stage A and in no patient in stage B.

Some neutralizing antibodies may not be detected by the assay. The available data are too limited to make definitive conclusions regarding immunogenicity and the effect on pharmacokinetics, safety, or efficacy of VYVGART HYTRULO.

### 13 Nonclinical Toxicology

### 13.1 Carcinogenesis, Mutagenesis, Impairment of Fertility

VYVGART HYTRULO for subcutaneous injection contains efgartigimod alfa and hyaluronidase [see Description (11)].

### Carcinogenesis and Mutagenesis

No studies have been conducted to assess the carcinogenic potential of efgartigimod alfa.

No studies have been conducted to assess the genotoxic potential of efgartigimod alfa.

No carcinogenicity or genotoxicity studies were conducted for human recombinant hyaluronidase.

### Impairment of Fertility

Intravenous administration of efgartigimod alfa (0, 30, or 100 mg/kg/day) to male and female rats prior to and during mating and continuing in females through gestation day 7 resulted in no adverse effects on fertility. Efgartigimod alfa exposures at the highest no-effect dose were approximately 12 times that in humans at the recommended human dose of 1008 mg.

There were no effects on reproductive tissues in monkeys following subcutaneous administration of hyaluronidase (human recombinant) doses up to approximately 1,200 times the dose of hyaluronidase at the recommended human dose (RHD) of VYVGART HYTRULO (1,008 mg efgartigimod alfa and 11,200 U hyaluronidase) on a U/kg basis for 39 weeks. No systemic exposure to hyaluronidase was observed at doses up to approximately 120 times the dose of hyaluronidase at the RHD of VYVGART HYTRULO, on a U/kg basis.

### 14 Clinical Studies

### 14.1 Generalized Myasthenia Gravis

Study 1 (described below) which established the effectiveness of efgartigimod alfa-fcab for the treatment of generalized myasthenia gravis (gMG) in adults who are AChR antibody positive was conducted with efgartigimod alfa-fcab intravenous formulation. In Study 2, VYVGART HYTRULO demonstrated a comparable pharmacodynamic effect on AChR antibody reduction as compared to the efgartigimod alfa-fcab intravenous formulation, which established the efficacy of VYVGART HYTRULO [see Clinical Pharmacology (12.2)].

### Study 1 (Efgartigimod Alfa-fcab Intravenous)

The efficacy of efgartigimod alfa-fcab intravenous (EFG IV) for the treatment of generalized myasthenia gravis (gMG) in adults who are AChR antibody positive was established in a 26-week, multicenter, randomized, double-blind, placebo-controlled trial (Study 1; NCT03669588).

Study 1 enrolled patients who met the following criteria at screening:
Myasthenia Gravis Foundation of America (MGFA) clinical classification class II to IV
MG-Activities of Daily Living (MG-ADL) total score of ≥5
On stable dose of MG therapy prior to screening, that included acetylcholinesterase (AChE) inhibitors, steroids, or non-steroidal immunosuppressive therapies (NSISTs), either in combination or alone
IgG levels of at least 6 g/L

A total of 167 patients were enrolled in Study 1 and were randomized to receive either EFG IV 10 mg/kg (1200 mg for those weighing 120 kg or more) (n=84) or placebo (n=83). Baseline characteristics were similar between treatment groups. Patients had a median age of 46 years at screening (range: 19 to 81 years) and a median time since diagnosis of 7 years. Seventy-one percent were female, and 84% were White. Median MG-ADL total score was 9, and median Quantitative Myasthenia Gravis (QMG) total score was 16. The majority of patients (n=65 for EFG IV; n=64 for placebo) were positive for AChR antibodies.

At baseline, over 80% of patients in each group received AChE inhibitors, over 70% in each treatment group received steroids, and approximately 60% in each treatment group received NSISTs, at stable doses.

Patients were treated with 10 mg/kg EFG IV administered as an intravenous infusion over one hour once weekly for 4 weeks. In patients weighing 120 kg or more, EFG IV was administered as 1200 mg per infusion. Subsequent treatment cycles were administered based on clinical evaluation, but no sooner than 50 days from the start of the previous treatment cycle.

The efficacy of EFG IV was measured using the Myasthenia Gravis-Specific Activities of Daily Living scale (MG-ADL) which assesses the impact of gMG on daily functions of 8 signs or symptoms that are typically affected in gMG. Each item is assessed on a 4-point scale where a score of 0 represents normal function and a score of 3 represents loss of ability to perform that function. A total score ranges from 0 to 24, with the higher scores indicating more impairment. In this study, an MG-ADL responder was defined as a patient with a 2-point or greater reduction in the total MG-ADL score compared to the treatment cycle baseline for at least 4 consecutive weeks, with the first reduction occurring no later than 1 week after the last infusion of the cycle.

The primary efficacy endpoint was the comparison of the percentage of MG-ADL responders during the first treatment cycle between treatment groups in the AChR-Ab positive population. A statistically significant difference favoring EFG IV was observed in the MG-ADL responder rate during the first treatment cycle [67.7% in the EFG IV-treated group vs 29.7% in the placebo-treated group (p<0.0001)].

The efficacy of EFG IV was also measured using the Quantitative Myasthenia Gravis (QMG) total score which is a 13-item categorical grading system that assesses muscle weakness. Each item is assessed on a 4-point scale where a score of 0 represents no weakness and a score of 3 represents severe weakness. A total possible score ranges from 0 to 39, where higher scores indicate more severe impairment. In this study, a QMG responder was defined as a patient who had a 3-point or greater reduction in the total QMG score compared to the treatment cycle baseline for at least 4 consecutive weeks, with the first reduction occurring no later than 1 week after last infusion of the cycle.

The secondary endpoint was the comparison of the percentage of QMG responders during the first treatment cycle between both treatment groups in the AChR-Ab positive patients. A statistically significant difference favoring EFG IV was observed in the QMG responder rate during the first treatment cycle [63.1% in the EFG IV-treated group vs 14.1% in the placebo-treated group (p<0.0001)].

The results are presented in Table 21.

TABLE 21
MG-ADL and QMG Responders During Cycle 1 in
AChR-Ab Positive Patients (mITT Analysis Set)

| | EFG IV | Placebo | P- | Odds Ratio |
|---|---|---|---|---|
| | n = 65 | n = 64 | value | (95% CI) |
| MG-ADL | 67.7 | 29.7 | <0.0001 | 4.951 (2.213, 11.528) |
| Responders | | | | |
| QMG Responders | 63.1 | 14.1 | <0.0001 | 10.842 (4.179, 31.200) |

EFG IV = Efgartigimod alfa-fcab intravenous; MG-ADL = Myasthenia Gravis Activities of Daily Living; QMG = Quantitative Myasthenia Gravis; mITT = modified intent-to-treat; n = number of patients for whom the observation was reported; CI = confidence interval.
Logistic regression stratified for AChR-Ab status (if applicable), Japanese/Non-Japanese and standard of care, with baseline MG-ADL as covariate/QMG as covariates.
Two-sided exact p-value.

### 14.2 Chronic Inflammatory Demyelinating Polyneuropathy

The efficacy of VYVGART HYTRULO for the treatment of adults with chronic inflammatory demyelinating polyneuropathy (CIDP) was established in a two stage, multicenter study (Study 3; NCT04281472).

Study 3 enrolled male and female patients age 18 years and older, who at the time of screening, had a documented diagnosis of definite or probable CIDP using the European Federation of Neurological Societies/Peripheral Nerve Society (EFNS/PNS; 2010) criteria for progressing or relapsing forms.

The Inflammatory Neuropathy Cause and Treatment disability score (INCAT) is a scale used to assess the impact of CIDP on daily upper and lower limb function, and is composed of the arm score and leg score (0 to 5 points for each). A total score on the INCAT ranges from 0 to 10 points with a higher number representing more disability. The adjusted INCAT (aINCAT) disability score, identical to the INCAT disability score but with changes in the upper limb function from 0 (normal) to 1 (minor symptoms) excluded, was used to assess efficacy for VYVGART HYTRULO for the treatment of CIDP.

### Stage A

In Stage A, a total of 322 patients received up to 12 once weekly subcutaneous injections of VYVGART HYTRULO 1008 mg/11,200 units until evidence of improvement occurred at two consecutive study visits. Improvement was defined as aINCAT improvement ≥1 point, I-RODS improvement ≥4 points, or mean grip strength improvement ≥8 kPa. Stage A included 228 patients currently receiving standard-of-care therapy and 94 patients who had either not received prior treatment for CIDP or were not treated with standard-of-care therapy for at least 6 months before study entry. Sixty-nine percent of patients (n=221) who had documented improvement at two consecutive visits during Stage A then entered Stage B.

### Stage B

In stage B, a total of 221 patients were randomized to receive once weekly subcutaneous injections of VYVGART HYTRULO 1008 mg/11,200 units (n=111) or placebo (n=110).

Baseline characteristics of patients in stage B were similar between treatment groups. Patients had a median age of 55 years (range: 20 to 82 years), a median time since CIDP diagnosis of 2.2 years and median INCAT score of 3.0. Sixty-four percent were male and 65% were White, 30% Asian, and 1% African American.

Stage B included 146 patients currently receiving standard-of-care therapy and 75 patients who had either not received prior treatment for CIDP or were not treated with standard-of-care therapy for at least 6 months before study entry.

The primary endpoint was the time to clinical deterioration defined as a 1-point increase in aINCAT at two consecutive visits or a ≥1-point increase in aINCAT at one visit. Patients who had clinical deterioration or completed week 48 in Stage B without clinical deterioration were withdrawn from the placebo-controlled portion of the study. The study stopped when 88 events of clinical deterioration occurred for the primary endpoint analysis.

Patients who received VYVGART HYTRULO experienced a longer time to clinical deterioration (i.e., increase of ≥1 point in aINCAT score) compared to patients who received placebo, which was statistically significant, as demonstrated by a hazard ratio of 0.394 [95% CI (0.253; 0.614) p<0.0001]. The results are presented in Table 19 and FIG. 3A.

Note: The time to clinical deterioration is defined as the time in days from the first VYVGART HYTRULO or placebo administration in Stage B to the first occurrence of either: an increase in aINCAT score of ≥1 point compared with Stage B baseline if confirmed at the next visit or an increase in aINCAT score of ≥1 point compared with Stage B baseline.

### 16 how Supplied/Storage and Handling

VYVGART HYTRULO (efgartigimod alfa and hyaluronidase-qvfc) injection is a preservative free, sterile, yellowish, clear to opalescent solution supplied as one single-dose vial per carton containing 1,008 mg efgartigimod alfa and 11,200 units hyaluronidase per 5.6 mL (180 mg/2,000 units per mL): (NDC 73475-3102-3).

Store VYVGART HYTRULO vials refrigerated at 2° C. to 8° C. (36° F. to 46° F.) in the original carton to protect from light until time of use. Do not freeze. Do not shake.

If needed, unopened vials may be stored in the original carton for up to 3 days at room temperature at 20° C. to 25° C. (68° F. to 77° F.) for a single period before administration or returned to refrigeration. Do not store the vial at room temperature more than one time. Record the date removed from and the date returned to the refrigerator on the carton.

### 17 Patient Counseling Information

### Infections

Instruct patients to communicate any history of infections to the healthcare provider and to contact their healthcare provider if they develop any symptoms of an infection. Advise patients to complete age-appropriate vaccines according to immunization guidelines prior to initiation of a new treatment cycle with VYVGART HYTRULO. Administration of live or live-attenuated vaccines is not recommended during treatment with VYVGART HYTRULO [see Warnings and Precautions (5.1)].

### Hypersensitivity Reactions

Inform patients that hypersensitivity reactions, including angioedema and anaphylaxis, have occurred in patients who were treated with efgartigimod alfa products. Inform patients about the signs and symptoms of these reactions, and advise patients to contact their healthcare provider immediately if these occur [see Warnings and Precautions (5.2)].

### Infusion-Related Reactions

Advise patients of the potential risk of infusion-related reactions, which can include hypertension, chills, shivering, and chest, abdominal, and back pain. [see Warnings and Precautions (5.3)].

### Pregnancy Registry

There is a pregnancy exposure registry that monitors pregnancy outcomes in women exposed to VYVGART HYTRULO during pregnancy. Encourage participation and advise patients about how they may enroll in the registry [see Use in Specific Populations (8.1)].

### Example 4: Label with Prescribing Information for VYVGART

The text below is from the proposed updated label under review by the European Medicines Agency with the prescribing information for VYVGART (efgartigimod alfa) solution for injection.

This medicinal product is subject to additional monitoring. This will allow quick identification of new safety information. Healthcare professionals are asked to report any suspected adverse reactions. See section 4.8 for how to report adverse reactions.

### 1. Name of the Medicinal Product

Vyvgart 1 000 mg solution for injection

### 2. Qualitative and Quantitative Composition

Each vial contains 1 000 mg of efgartigimod alfa in 5.6 mL (180 mg/mL).

Efgartigimod alfa is a human recombinant immunoglobulin G1 (IgG1)-derived Fc fragment produced in Chinese hamster ovary (CHO) cells by recombinant DNA technology.

For the full list of excipients, see section 6.1.

### 3. Pharmaceutical Form

Solution for injection
Yellowish, clear to opalescent, pH 6.0.

### 4. Clinical Particulars

### 4.1 Therapeutic Indications

Vyvgart is indicated
as an addon to standard therapy for the treatment of adult patients with generalised Myasthenia Gravis (gMG) who are antiacetylcholine receptor (AChR) antibody positive.
for the treatment of adult patients with chronic inflammatory demyelinating polyneuropathy (CIDP) with active disease despite treatment with corticosteroids or immunoglobulins.

### 4.2 Posology and Method of Administration

Treatment must be initiated and supervised by a physician experienced in the management of patients with neuromuscular disorders.

### Posology

### Generalised Myasthenia Gravis

The first treatment cycle and first administration of the second treatment cycle must be administered either by or under the supervision of a healthcare professional. Subsequent treatment should be administered by a healthcare professional or may be administered at home by a patient or caregiver after adequate training in the subcutaneous injection technique.

The recommended dose is 1 000 mg to be administered subcutaneously in cycles of once weekly injections for 4 weeks. Subsequent treatment cycles should be administered according to clinical evaluation. The frequency of treatment cycles may vary by patient (see section 5.1).

In the clinical development program, the earliest time to initiate a subsequent treatment cycle was 7 weeks from the initial infusion of the previous cycle. The safety of initiating subsequent cycles sooner than 7 weeks from the start of the previous treatment cycle has not been established.

For patients currently receiving efgartigimod alfa intravenously, the solution for subcutaneous injection may be used as an alternative. It is recommended to switch between formulations at the start of a new treatment cycle. No safety and efficacy data in patients switching formulations during the same cycle is available.

### Chronic Inflammatory Demyelinating Polyneuropathy

The initial injection must be administered either by or under the supervision of a healthcare professional. Subsequent injections should be administered by a healthcare professional or may be administered at home by a patient or caregiver after adequate training in the subcutaneous injection technique and if the healthcare professional determines that this is appropriate and safe (see Section 4.4).

### OR

The first 4 injections must be administered either by or under the supervision of a healthcare professional. Subsequent injections should be administered by a healthcare professional or may be administered at home by a patient or caregiver after adequate training in the subcutaneous injection technique.

The recommended dose is 1 000 mg efgartigimod alfa administered subcutaneously as once-weekly injections.

Treatment is initiated with a weekly dose regimen and may be adjusted to every other week based on clinical evaluation. In case of worsening of symptoms, administration of once-weekly injections should be resumed.

For those patients currently receiving other CIDP therapies, Vyvgart treatment should preferably be initiated before the clinical effect of these prior therapies starts to decrease.

Clinical response is usually achieved within 3 months of initiation of treatment with efgartigimod alfa subcutaneous.

### Missed Dose

An interval of at least 3 days should be observed between two consecutive administrations. When administrations cannot be done at the scheduled time point, they should be performed as soon as possible and at least 3 days ahead of the following administration. If there are less than 3 days to the next administration, the missed dose should be skipped and the next dose should be administered at the scheduled time point.

### Special Populations

### Elderly

No dose adjustment is required in patients aged 65 years and older (see section 5.2).

### Renal Impairment

Limited safety and efficacy data in patients with mild renal impairment is available, no dose adjustment is required for patients with mild renal impairment. There is very limited safety and efficacy data in patients with moderate or severe renal impairment (see section 5.2).

### Hepatic Impairment

No data in patients with hepatic impairment are available. No dose adjustment is required in patients with hepatic impairment (see section 5.2).

### Paediatric Population

The safety and efficacy of efgartigimod alfa in paediatric population have not yet been established. No data are available.

### Method of Administration

This medicinal product should only be administered via subcutaneous injection. Do not administer intravenously.

After removing the vial from the refrigerator, wait for at least 15 minutes before injecting to allow the solution to reach room temperature. Use aseptic technique when preparing and administering the medicinal product solution. Do not shake the vial.

The solution for injection can be administered using a polypropylene syringe, stainless steel transfer needles and polyvinyl chloride winged infusion set, with a maximum priming volume of 0.4 mL.

Withdraw the entire content of the efgartigimod alfa solution from the vial using a transfer needle.

Change the needle on the syringe to the winged infusion set.

Prior to administration, the volume in the syringe should be adjusted to 5.6 mL.

During administration of the initial doses 5 injections of efgartigimod alfa (see section 4.2), appropriate treatment for injection and hypersensitivity-related reactions should be readily available (see section 4.4). The recommended injection sites (abdomen) should be rotated and injections should never be given into moles, scars, or areas where the skin is tender, bruised, red or hard. The volume of 5.6 mL should be injected over 30 to 90 seconds. The injection may be slowed if the patient experiences discomfort.

The first self-administration must always be conducted under the supervision of a healthcare professional. After adequate training in subcutaneous injection technique, patients or caregivers may inject the medicinal product at home if a healthcare professional determines that it is appropriate. Patients or caregivers should be instructed to inject Vyvgart according to the directions provided in the package leaflet.

For comprehensive instructions for the administration of the medicinal product, please refer to the Instructions for Use in the package leaflet.

### 4.3 Contraindications

Hypersensitivity to the active substance or to any of the excipients listed in section 6.1.

### 4.4 Special Warnings and Precautions for Use

### Traceability

In order to improve the traceability of biological medicinal products, the name and the batch number of the administered product should be clearly recorded.

Myasthenia Gravis Foundation of America (MGFA) Class V patients

Treatment with efgartigimod alfa in patients with MGFA Class V (i.e. myasthenic crisis), defined as intubation with or without mechanical ventilation except in the setting of routine postoperative care, has not been studied. The sequence of therapy initiation between established therapies for MG crisis and efgartigimod alfa, and their potential interactions, should be considered (see section 4.5).

### Infections

As efgartigimod alfa causes transient reduction in IgG levels the risk of infections may increase (see sections 4.8 and 5.1). The most common infections observed in clinical trials were upper respiratory tract infections and urinary tract infections (see section 4.8). Patients should be monitored for clinical signs and symptoms of infections during treatment with Vyvgart. In patients with an active infection, the benefit-risk of maintaining or withholding treatment with efgartigimod alfa should be considered until the infection has resolved. If serious infections occur, delaying treatment with efgartigimod alfa should be considered until the infection has resolved.

### Injection Reactions and Hypersensitivity Reactions

Injection reactions such as rash or pruritus were reported in the clinical trials (see section 4.8). These were mild to moderate. Cases of anaphylactic reaction have been reported with efgartigimod alfa intravenous in the post-marketing setting. The first injections must be administered under the supervision of a healthcare professional (see section 4.2). Patients should be monitored for 30 minutes after administration for clinical signs and symptoms of injection reactions. Should a reaction occur and based on the severity of the reaction, appropriate supportive measures should be initiated. Subsequent injections may be cautiously administered, based on clinical evaluation.

If an anaphylactic reaction is suspected, administration of Vyvgart should be immediately discontinued and appropriate medical treatment initiated. Patients should be informed of the signs and symptoms of hypersensitivity and anaphylactic reactions and advised to contact their healthcare professional immediately should they occur.

### Immunisations

All vaccines should be administered according to immunisation guidelines.

The safety of immunisation with live or live-attenuated vaccines and the response to immunisation with these vaccines during treatment with efgartigimod alfa are unknown. For patients that are being treated with efgartigimod alfa, vaccination with live or live-attenuated vaccines is generally not recommended. If vaccination with live or live-attenuated vaccines is required, these vaccines should be administered at least 4 weeks before treatment and at least 2 weeks after the last dose of efgartigimod alfa.

Other vaccines may be administered as needed at any time during treatment with efgartigimod alfa.

### Immunogenicity

In the active-controlled study ARGX-113-2001, pre-existing antibodies that bind to efgartigimod alfa were detected in 12/110 (11%) patients with gMG. Anti-efgartigimod alfa antibodies were detected in 19/55 (35%) patients treated with efgartigimod alfa subcutaneous compared to 11/55 (20%) patients treated with the intravenous formulation. Neutralising antibodies were detected in 2 (4%) patients treated with efgartigimod alfa subcutaneous and 2 (4%) patients treated with efgartigimod alfa intravenous.

In study ARGX-113-1802, pre-existing antibodies that bind to efgartigimod alfa were detected in 13/317 (4.1%) patients with CIDP. Anti-efgartigimod alfa antibodies were detected in 20/317 (6.3%) of patients treated with in Stage A, and in 2/111 (1.8%) of patients treated with in Stage B. Neutralising antibodies were detected in 1 (0.3%) patient in Stage A only.

The impact of antibodies to efgartigimod alfa on clinical efficacy or safety, pharmacokinetics and pharmacodynamic cannot be assessed given the low incidence of neutralizing antibodies.

### Immunosuppressant and Anticholinesterase Therapies

When non-steroidal immunosuppressants, corticosteroids and anticholinesterase therapies are decreased or discontinued, patients should be monitored closely for signs of disease exacerbation.

### Sodium Content

This medicinal product contains less than 1 mmol sodium (23 mg) per vial, that is to say essentially ‘sodium-free’.

4.5 Interaction with Other Medicinal Products and Other Forms of Interaction

No interaction studies have been performed.

Efgartigimod alfa may decrease concentrations of compounds that bind to the human neonatal Fc Receptor (FcRn), i.e., immunoglobulin products, monoclonal antibodies, or antibody derivatives containing the human Fc domain of the IgG subclass. If possible, it is recommended to postpone the initiation of treatment with these products to 2 weeks after the last dose of Vyvgart. As a precaution, patients receiving Vyvgart while on treatment with these products should be closely monitored for the intended efficacy response of those products.

Plasma exchange, immunoadsorption, and plasmapheresis may reduce circulating levels of efgartigimod alfa.

All vaccines should be administered according to immunisation guidelines.

The potential interaction with vaccines was studied in a nonclinical model using Keyhole limpet hemocyanin (KLH) as the antigen. The weekly administration of 100 mg/kg to monkeys did not impact the immune response to KLH immunisation.

For patients that are being treated with efgartigimod alfa, vaccination with live or live attenuated vaccines is generally not recommended. If vaccination with live or live attenuated vaccines is required, these vaccines should be administered at least 4 weeks before treatment and at least 2 weeks after the last dose of efgartigimod alfa (see section 4.4).

### 4.6 Fertility, Pregnancy and Lactation

### Pregnancy

There is no available data on the use of efgartigimod alfa during pregnancy. Antibodies including therapeutic monoclonal antibodies are known to be actively transported across the placenta (after 30 weeks of gestation) by binding to FcRn.

Efgartigimod alfa may be transmitted from the mother to the developing foetus. As efgartigimod alfa is expected to reduce maternal antibody levels, and is also expected to inhibit the transfer of maternal antibodies to the foetus, reduction in passive protection to the newborn is anticipated. Therefore, risks and benefits of administering live/live attenuated vaccines to infants exposed to efgartigimod alfa in utero should be considered (see section 4.4).

Treatment of pregnant women with Vyvgart should only be considered if the clinical benefit outweighs the risks.

### Breast-Feeding

There is no information regarding the presence of efgartigimod alfa in human milk, the effects on the breastfed child or the effects on milk production. Animal studies on the transfer of efgartigimod alfa into milk have not been conducted, and therefore, excretion into maternal milk cannot be excluded. Maternal IgG is known to be present in human milk. Treatment of lactating women with efgartigimod alfa should only be considered if the clinical benefit outweighs the risks.

### Fertility

There is no available data on the effect of efgartigimod alfa on fertility in humans. Animal studies showed no impact of efgartigimod alfa on male and female fertility parameters (see section 5.3).

### 4.7 Effects on Ability to Drive and Use Machines

Vyvgart has no or negligible influence on the ability to drive and use machines.

### 4.8 Undesirable Effects

### Summary of the Safety Profile

The most frequently observed adverse reactions were injection site reactions (33%), upper respiratory tract infections (10.7%) and urinary tract infections (9.5%).

The overall safety profile of Vyvgart subcutaneous for both cyclic and continuous dose regimens was consistent with the known safety profile of the intravenous formulation.

### Tabulated List of Adverse Reactions

Adverse reactions described in this section were identified in clinical trials and from post-marketing reports. These reactions are presented by system organ class and preferred term. Frequency categories are defined as: very common (≥1/10), common (≥1/100 to <1/10), uncommon (≥1/1 000 to <1/100), rare (≥1/10 000 to <1/1 000) or not known (cannot be estimated from the available data). Within each frequency grouping, adverse reactions are presented in order of decreasing seriousness.

TABLE 22
Adverse Reactions

| | | Frequency |
|---|---|---|
| System organ class | Adverse reaction | category |
| Infections and | Upper respiratory tract | Very |
| infestations* | infections | common |
| | Urinary tract infections | Common |
| | Bronchitis | Common |
| Immune system disorders | Anaphylactic reaction ^{a} | Not known |
| Musculoskeletal and | Myalgia | Common |
| connective tissue | | |
| disorders | | |
| General disorders and | Injection site reactions ^{b, c} | Very |
| administration site | | common |
| conditions* | | |
| Injury, poisoning and | Procedural headache ^{d} | Common |
| procedural complications* | | |

*See paragraph “Description of selected adverse reactions”
^{a} From spontaneous post-marketing reporting with intravenous route of administration
^{b} Subcutaneous administration only.
^{c} (e.g. injection site rash, injection site erythema, injection site pruritus, injection site pain)
^{d} Intravenous administration only.

### Description of Selected Adverse Reactions

### Injection Site Reactions

In the pooled dataset from two clinical studies in gMG with efgartigimod alfa subcutaneous (n=168), all injection site reactions were mild to moderate in severity and did not lead to treatment discontinuation. 44.0% (n=74)) of patients experienced an injection site reaction. Injection site reactions occurred within 24 hours after administration in 78.4% (58/74) of patients and resolved without treatment in 85.1% (63/74) of the patients. The incidence of injection site reactions was the highest in the first treatment cycle, reported in 36.3% (61/168) of patients during the first treatment cycle and decreased to 20.1% (30/149), 15.4% (18/117) and 12.5% (10/80) of patients with the second, third and fourth treatment cycle. In a pooled dataset from 2 clinical studies in patients with CIDP who received continuous weekly administration of efgartigimod alfa subcutaneous the incidence of injection site reactions was 26% (61/235). Analysis by 3-month intervals showed that the percentage of participants with injection-site reactions was highest in the first 3 months of treatment (73 [22.2%] participants) and decreased in subsequent 3-month intervals (range: 0 to 17 [6.8%] participants).

### Infections

In the gMG ARGX 113 1704 placebo-controlled study with efgartigimod alfa intravenous, the most frequently reported adverse reactions were infections, and the most reported infections were upper respiratory tract infections, (10.7% [n=9] of patients treated with efgartigimod alfa intravenous and 4.8% [n=4] of patients treated with placebo) and urinary tract infections (9.5% [n=8] of patients treated with efgartigimod alfa intravenous and 4.8% [n=4] of patients treated with placebo). These infections were mild to moderate in severity in patients who received efgartigimod alfa intravenous (≤Grade 2 according to the Common Terminology Criteria for Adverse Events). Overall, treatment emergent infections were reported in 46.4% (n=39) of patients treated with efgartigimod alfa intravenous and 37.3% (n=31) of patients treated with placebo. The median time from treatment initiation to emergence of infections was 6 weeks. Incidence of infections did not increase with subsequent treatment cycles. Treatment discontinuation or temporary interruption of treatment due to an infection occurred in less than 2% of patients. In the ARGX-113-1802 (Stage B) placebo-controlled study in patients with CIDP, continuous weekly administration of efgartigimod alfa subcutaneous was not associated with any increase in the incidence of infections (31.5% [35/111] in the efgartigimod alfa subcutaneous group and 33.6% [37/110] in the placebo group).

### Procedural Headache

Procedural headache was reported in 4.8% of the patients treated with efgartigimod alfa intravenous and 1.2% of patients treated with placebo. Procedural headache was reported when a headache was judged to be temporally related to the intravenous infusion of efgartigimod alfa. All were mild or moderate except one event which was reported as severe (Grade 3).

All other adverse reactions were mild or moderate with the exception of one case of myalgia (Grade 3).

### Reporting of Suspected Adverse Reactions

Reporting suspected adverse reactions after authorisation of the medicinal product is important. It allows continued monitoring of the benefit/risk balance of the medicinal product. Healthcare professionals are asked to report any suspected adverse reactions via the national reporting system listed in Appendix V.

### 4.9 Overdose

There are no known specific signs and symptoms of overdose with efgartigimod alfa. In the event of an overdose the adverse events that may occur are not expected to be different from those that may be observed at the recommended dose. Patients should be monitored for adverse reactions, and appropriate symptomatic and supportive treatment initiated. There is no specific antidote for overdose with efgartigimod alfa.

### 5. Pharmacological Properties

### 5.1 Pharmacodynamic Properties

Pharmacotherapeutic group: Immunosuppressants, selective immunosuppressants, ATC code: L04AA58

### Mechanism of Action

Efgartigimod alfa is a human IgG1 antibody fragment engineered for increased affinity to the neonatal Fc Receptor (FcRn). Efgartigimod alfa binds to FcRn, resulting in a reduction in the levels of circulating IgG including pathogenic IgG autoantibodies. Efgartigimod alfa does not affect the levels of other immunoglobulins (IgA, IgD, IgE or IgM), and does not reduce those of albumin.

IgG autoantibodies are the underlying cause of the pathogenesis of IgG mediated autoimmune diseases.

In MG these impair neuromuscular transmission by binding to acetylcholine receptors (AChR), muscle specific tyrosine kinase (MuSK) or low density lipoprotein receptor-related protein 4 (LRP4).

In CIDP, several lines of evidence point to the key role of IgG autoantibodies in the pathogenesis of this disease. This includes the demonstration of autoreactive IgG antibodies against components of myelinated nerves, passive transfer of CIDP symptoms to animal models using sera or IgG's from patients with CIDP, and the effectiveness of plasma exchange and immunoadsorption for treating patients with CIDP.

### Pharmacodynamic Effects

### Intravenous Formulation

In the ARGX 113 1704 double blind placebo controlled study in gMG patients, efgartigimod alfa 10 mg/kg administered once weekly for 4 weeks decreased serum IgG levels and AChR autoantibody (AChR Ab) levels. Maximum mean percentage decrease in total IgG levels compared to baseline reached 61% one week after the last infusion of the initial treatment cycle and returned to baseline levels 9 weeks after the last infusion. Similar effects were also observed for all subtypes of IgG. Decrease in AChR-Ab levels followed a similar time course with maximum mean percentage decrease of 58% one week after the last infusion and return to baseline levels 7 weeks after the last infusion. Similar changes were observed during the second cycle of the study. Subcutaneous formulation

In the ARGX-113-2001 study, decreases in AChR Ab levels followed a comparable time course as total IgG levels and were similar between the efgartigimod alfa subcutaneous and intravenous groups. Maximum mean percentage decreases in AChR Ab levels of 62.2% and 59.6% were observed one week after the last administration in the efgartigimod alfa subcutaneous and intravenous groups, respectively. For both the efgartigimod alfa subcutaneous and intravenous groups, decrease in total IgG and AChR Ab levels were associated with a clinical response, as measured by the change from baseline in MG ADL total score.

In the ARGX-113-1802 study, in patients with CIDP receiving continuous once-weekly administration of efgartigimod alfa subcutaneous at 1 000 mg, the mean percent change from baseline in total IgG levels was sustained from Week 4 throughout the treatment period (mean percentage reduction from baseline ranging between 66.8 to 71.6%).

### Clinical Efficacy and Safety

### Generalised Myasthenia Gravis

### Intravenous Formulation

Efficacy of efgartigimod alfa for the treatment of adults with generalised Myasthenia Gravis (gMG) was studied in a 26 week, multicentre randomised double blind placebo controlled trial (ARGX 113 1704).

In this study, patients had to meet the following main criteria at screening:
Myasthenia Gravis Foundation of America (MGFA) clinical classification class II, III or IV;
Patients with either positive or negative serologic tests for antibodies to AChR;
MG Activities of Daily Living (MG ADL) total score of ≥5;
On stable doses of MG therapy prior to screening, that included acetylcholinesterase (AChE) inhibitors, steroids or non steroidal immunosuppressive therapy (NSIST), either in combination or alone [NSISTs included but were not limited to azathioprine, methotrexate, cyclosporine, tacrolimus, mycophenolate mofetil, and cyclophosphamide];
IgG levels of at least 6 g/L.

Patients with MGFA Class V gMG; patients with documented lack of clinical response to PLEX; patients treated with PLEX, IVIg one month and monoclonal antibodies six months prior to starting treatment; and patients with active (acute or chronic) hepatitis B infection, hepatitis C seropositivity, and diagnosis of AIDS, were excluded from the trials.

A total of 167 patients were enrolled in the study and were randomised to either efgartigimod alfa intravenous (n=84) or placebo (n=83). Baseline characteristics were similar between treatment groups, including median age at diagnosis [45 (19 81) years], gender [most were female; 75% (efgartigimod alfa) versus 66% (placebo)], race [most patients were white; 84.4%] and median time since diagnosis [8.2 years (efgartigimod alfa) and 6.9 years (placebo)].

The majority of patients (77% in each group) tested positive for antibodies to AChR (AChR Ab) and 23% of patients tested negative for AChR Ab.

During the study, over 80% of patients in each group received AChE inhibitors, over 70% in each treatment group received steroids, and approximately 60% in each treatment group received NSISTs, at stable doses. At study entry, approximately 30% of patients in each treatment group had no previous exposure to NSISTs.

Median MG ADL total score was 9.0 in both treatment groups, and median Quantitative Myasthenia Gravis (QMG) total score was 17 and 16 in the efgartigimod alfa and placebo groups, respectively.

Patients were treated with efgartigimod alfa intravenous 10 mg/kg administered once weekly for 4 weeks and received a maximum of 3 treatment cycles.

The efficacy of efgartigimod alfa was measured using the Myasthenia Gravis Specific Activities of Daily Living scale (MG ADL) which assesses the impact of gMG on daily functions. A total score ranges from 0 to 24 with the higher scores indicating more impairment. In this study, an MG ADL responder was a patient with ≥2 point reduction in the total MG ADL score compared to the treatment cycle baseline, for at least 4 consecutive weeks with the first reduction occurring no later than 1 week after the last infusion of the cycle.

The efficacy of efgartigimod alfa was also measured using the QMG total score which is a grading system that assesses muscle weakness with a total possible score of 0 to 39 where higher scores indicate more severe impairment. In this study, a QMG responder was a patient who had a ≥3 point reduction in the total QMG score compared to the treatment cycle baseline, for at least 4 consecutive weeks with the first reduction occurring no later than 1 week after last infusion of the cycle.

The primary efficacy endpoint was the comparison of the percentage of MG ADL responders during the first treatment cycle (C1) between treatment groups in the AChR Ab seropositive population.

A key secondary endpoint was the comparison of the percentage of QMG responders during C1 between both treatment groups in the AChR Ab seropositive patients.

Analyses show that during the second treatment cycle (see Table 23) MG-ADL responder rates were similar to those during the first treatment cycle (see Table 21).

TABLE 23
MG-ADL and QMG responders during cycle 2 in AChR-Ab
seropositive population (mITT analysis set)

| | | Efgartigimod alfa | Placebo |
|---|---|---|---|
| | Population | n/N (%) | n/N (%) |
| MG-ADL | AChR-Ab | 36/51 (70.6) | 11/43 (25.6) |
| | seropositive | | |
| QMG | AChR-Ab | 24/51 (47.1) | 5/43 (11.6) |
| | seropositive | | |

AChR-Ab = acetylcholine receptor-antibody; MG-ADL = Myasthenia Gravis Activities of Daily Living; QMG = Quantitative Myasthenia Gravis; mITT = modified intent-to-treat; n = number of patients for whom the observation was reported; N = number of patients in the analysis set.

Exploratory data shows that onset of response was observed within 2 weeks of initial infusion in 37/44 (84%) patients treated with efgartigimod alfa intravenous in the AChR-Ab seropositive MG-ADL responders.

In the double-blind placebo-controlled study (ARGX-113-1704), the earliest possible time to initiating the subsequent treatment cycle was 8 weeks after the initial infusion of the first treatment cycle. In the overall population the mean time to the second treatment cycle in the efgartigimod alfa intravenous group was 13 weeks (SD 5.5 weeks) and the median time was 10 weeks (8-26 weeks) from the initial infusion of the first treatment cycle. In the open-label extension study (ARGX-113-1705) the earliest possible time of initiation of the subsequent treatment cycles was 7 weeks.

In patients that responded to treatment, the duration of clinical improvement was 5 weeks in 5/44 (11%) patients, 6-7 weeks in 14/44 (32%) of patients, 8-11 weeks in 10/44 (23%) patients and 12 weeks or more in 15/44 (34%) patients.

### Subcutaneous Formulation

A 10-week, randomised, open-label, parallel-group, multicentre study (ARGX-113-2001) was conducted in adult patients with gMG to evaluate the non-inferiority of the pharmacodynamic effect of efgartigimod alfa subcutaneous compared to efgartigimod alfa intravenous. The main inclusion and exclusion criteria were the same as in study ARGX-113-1704.

A total of 110 patients were randomised and received one cycle of once weekly administrations for 4 weeks, of either efgartigimod alfa subcutaneous 1 000 mg (n=55) or efgartigimod alfa intravenous 10 mg/kg (n=55). The majority of patients were positive for antibodies to AChR (AChR-Ab): 45 patients (82%) in efgartigimod alfa subcutaneous group and 46 patients (84%) in efgartigimod alfa intravenous group. All patients were on stable doses of MG therapy prior to screening, that included AChE inhibitors, steroids or NSISTs, either in combination or alone.

Baseline characteristics were similar between treatment groups.

During the study, over 80% of patients in each group received AChE inhibitors, over 60% of patients in each group received steroids and about 40% in each treatment group received NSISTs, at stable doses. At study entry, approximately 56% of patients in each treatment group had no previous exposure to NSISTs.

The primary endpoint was the comparison of the percent reduction in total IgG levels from baseline at day 29 between treatment groups in the overall population. The results in the AChR-Ab seropositive population demonstrates non-inferiority of efgartigimod alfa subcutaneous compared to efgartigimod alfa intravenous (see Table 24).

TABLE 24
ANCOVA analysis of percent change from baseline in total IgG level
at day 29 in AChR-Ab seropositive population (mITT analysis set)

| | | | | | | Difference | | |
|---|---|---|---|---|---|---|---|---|
| | | | | | | Efgartigimod alfa SC − | | |
| | | | | | | Efgartigimod alfa IV | | |
| Efgartigimod alfa SC | | | Efgartigimod alfa IV | | | LS of Mean | | |
| N | LS Mean | 95% CI | N | LS Mean | 95% CI | difference | 95% CI | p-value |
| 41 | −66.9 | −69.78, −64.02 | 43 | −62.4 | −65.22, −59.59 | −4.5 | −8.53, −0.46 | <0.0001 |

AChR-Ab = acetylcholine receptor-antibody;
ANCOVA = analysis of covariance;
CI = confidence interval;
SC = subcutaneous;
IV = intravenous;
LS = least squares;
mITT = modified intent-to-treatment analysis set;
N = number of patients per group that were included in the ANCOVA analysis

Efficacy secondary endpoints were comparisons of the percentage of MG-ADL and QMG responders, as defined in study ARGX-113-1704, between both treatment groups. The results in AChR-Ab seropositive population are presented in Table 25.

TABLE 25
MG-ADL and QMG responders at day 29 in AChR-Ab
seropositive population (mITT analysis set)

| | Efgartigimod | Efgartigimod | Difference |
|---|---|---|---|
| | alfa SC | alfa IV | Efgartigimod alfa SC − |
| | n/N (%) | n/N (%) | Efgartigimod alfa IV (95% CI) |
| MG-ADL | 32/45 (71.1) | 33/46 (71.7) | −0.6 (−19.2 to 17.9) |
| QMG | 31/45 (68.9) | 24/45 (53.3) | 15.6 (−4.3 to 35.4) |

AChR-Ab = acetylcholine receptor-antibody;
MG-ADL = Myasthenia Gravis Activities of Daily Living;
QMG = Quantitative Myasthenia Gravis;
SC = subcutaneous;
IV = intravenous;
mITT = modified intent-to-treat;
n = number of patients for whom the observation was reported;
N = number of patients in the analysis set;
CI = confidence interval

Exploratory data shows that onset of response was observed within 2 weeks of initial administration in 28/32 (88%) patients treated with efgartigimod alfa subcutaneous and 27/33 (82%) patients treated with efgartigimod alfa intravenous in the AChR-Ab seropositive MG-ADL responders.

### Chronic Inflammatory Demyelinating Polyneuropathy

The efficacy of efgartigimod alfa subcutaneous for the treatment of adults with CIDP was demonstrated in a prospective, multicentre study ARGX-113-1802 conducted in 2 treatment stages: an open-label stage A and a randomized-withdrawal, double-blinded, placebo-controlled stage B.

Patients had been either on or off CIDP treatment during 6 months prior to study entry, and those on prior CIDP treatment entered a treatment-free run-in period. Patients who demonstrated evidence of clinically meaningful deterioration then entered Stage A of the study.

A total of 322 patients were enrolled in Stage A. Patients received up to 12 once weekly injections of efgartigimod alfa subcutaneous 1 000 mg until evidence of clinical improvement (ECI) occurred at 2 consecutive study visits. Subsequently, the patients with confirmed ECI entered Stage B of the study and were randomised to receive weekly administrations of either efgartigimod alfa subcutaneous (111 patients) or placebo (110 patients who were either on or off CIDP treatment within 6 months prior to the study entry

Baseline characteristics of stage B were similar between treatment groups. Patients had a median age of 55 years (range: 20 to 82 years), a median time since CIDP diagnosis of 2.2 years and median INCAT score of 3.0. Sixty-four percent were male and 65% were White.

Patients were stratified based on the most recent CIDP treatment received within 6 months before study entry: 35.3% of patients received no CIDP treatment, 21.3% of patients received corticosteroids and 43.4% of patients received immunoglobulins.

In stage A, the primary endpoint was the percentage of responders defined as patients achieving confirmed ECI. The primary endpoint was met in 66.5% of patients; further details are presented in Table 18.

A secondary endpoint in Stage A was the time to the first confirmed ECI. Week 4 was the earliest time point at which ECI criteria could be met. At that time point, up to 40% of patients achieved ECI. Based on an additional pre-specified analysis, 25% of patients showed clinically relevant improvement after 9 days in at least one of 3 parameters (aINCAT, I-RODS or Grip Strength).

The majority of patients achieved confirmed ECI across all prior CIDP medication groups.

In stage B, the primary endpoint was defined as the time to the occurrence of the first evidence of clinical deterioration (≥1-point increase in aINCAT compared to stage B baseline, which was confirmed at a consecutive visit after the first 1-point increase in aINCAT or, if not confirmed, a ≥2-point increase in aINCAT compared to stage B baseline). Patients who received efgartigimod alfa subcutaneous remained relapse-free (i.e., no clinical deterioration) significantly longer compared to patients who received placebo, as demonstrated by a hazard ratio of 0.394 [95% CI (0.253; 0.614) p=0.000039] which represents a 61% risk reduction for deterioration in patients treated with efgartigimod alfa. The results are presented in Table 19 and FIG. 3A.

Analyses were repeated in a pre-specified sub-group of patients who were on CIDP treatment within 6 months prior to study entry and had a CDAS score of ≥4 prior to screening (N=139). Evidence of clinical improvement was observed in 67.6% (94/139) of patients [95% CI (59.2; 75.3)] in stage A of the study, and the median time to initial confirmed ECI was 31 days [95% CI (23.0-43.0)]. Patients who received efgartigimod alfa subcutaneous during stage B of the study remained relapse-free (i.e., no clinical deterioration) significantly longer compared to patients who received placebo, as demonstrated by a hazard ratio of 0.269 [95% CI (0.138; 0.523) nominal p=0.0001] which represents a 73% risk reduction for deterioration in patients treated with efgartigimod alfa.

### Paediatric Population

The European Medicines Agency has deferred the obligation to submit the results of studies with Vyvgart in one or more subsets of the paediatric population in treatment of myasthenia gravis (see section 4.2 for information on paediatric use).

The European Medicines Agency has waived the obligation to submit the results of studies with Vyvgart in all subsets of the paediatric population in CIDP (see section 4.2 for information on paediatric use).

### 5.2 Pharmacokinetic Properties

### Absorption

Based upon population PK data analysis, the estimated bioavailability with efgartigimod alfa 1 000 mg subcutaneous is 77%.

The mean C_{trough} after 4 once weekly administrations with efgartigimod alfa 1 000 mg subcutaneous and efgartigimod alfa 10 mg/kg intravenous were 22.0 μg/mL (37% CV) and 14.9 μg/mL (43% CV), respectively. The AUC_{0-168 h} of efgartigimod alfa after administration of one treatment cycle with 1 000 mg subcutaneous and 10 mg/kg intravenous were comparable.

In patients receiving continuous subcutaneous administration of efgartigimod alfa 1 000 mg once weekly, mean C_{trough} ranged from 14.9 to 20.1 mcg/mL.

### Distribution

Based upon population PK data analysis in healthy subjects and patients the volume of distribution is 18 L.

### Biotransformation

Efgartigimod alfa is expected to be degraded by proteolytic enzymes into small peptides and amino acids.

### Elimination

The terminal half-life is 80 to 120 hours (3 to 5 days). Based upon population PK data analysis, the clearance is 0.128 L/h. The molecular weight of efgartigimod alfa is approximately 54 kDa, which is at the boundary of molecules that are renally filtered.

### Linearity/Non-Linearity

The pharmacokinetics profile of efgartigimod alfa is linear, independent of dose or time, with minimal accumulation.

### Special Populations

### Age, Gender, Race and Bodyweight

The pharmacokinetics of efgartigimod alfa were not affected by age (19-84 years), gender, race and bodyweight.

### Renal Impairment

No dedicated pharmacokinetic studies have been performed in patients with renal impairment.

The effect of renal function marker estimated glomerular filtration rate [eGFR] as a covariate in a population pharmacokinetic analysis showed an increase in exposure (11% to 21%) in patients with mild renal impairment (eGFR 60-89 mL/min/1.73 m²). No specific dose adjustment is recommended in patients with mild renal impairment.

There is insufficient data on the impact of moderate renal impairment (eGFR 30-59 mL/min/1.73 m²) and severe renal impairment (eGFR <30 mL/min/1.73 m²) on efgartigimod alfa pharmacokinetic parameters.

### Hepatic Impairment

No dedicated pharmacokinetic study has been performed in patients with hepatic impairment.

The effect of hepatic function markers as covariates in a population pharmacokinetic analysis did not show any impact on the pharmacokinetics of efgartigimod alfa.

### 5.3 Preclinical Safety Data

Non-clinical data reveal no special hazard for humans based on conventional studies of safety pharmacology and repeated dose toxicity.

In reproduction studies in rats and rabbits, intravenous administration of efgartigimod alfa did not result in adverse effects on fertility and pregnancy nor were teratogenic effects observed up to dose levels corresponding to 11-fold (rats) and 56-fold (rabbits) a human 10 mg/kg exposure based on AUC.

### Carcinogenicity and Genotoxicity

No studies have been conducted to assess the carcinogenic and genotoxic potential of efgartigimod alfa.

Hyaluronidase is found in most tissues of the human body. Non-clinical data for recombinant human hyaluronidase reveal no special hazard for humans based on conventional studies of repeated dose toxicity including safety pharmacology endpoints. Reproductive toxicology studies with rHuPH20 revealed embryofoetal toxicity in mice at high systemic exposure, but did not show teratogenic potential.

### 6. Pharmaceutical Particulars

### 6.1 List of Excipients

Recombinant human hyaluronidase (rHuPH20)
L-histidine
L-histidine hydrochloride monohydrate
L-methionine,
Polysorbate 20
Sodium chloride
Sucrose
Water for injections

### 6.2 Incompatibilities

In the absence of compatibility studies, this medicinal product must not be mixed with other medicinal products.

### 6.3 Shelf Life

18 months

If needed, unopened vials may be stored at room temperature (up to 30° C.) for up to 3 days. After storage at room temperature, unopened vials may be returned to the refrigerator. If stored out of and then returned to refrigeration, the total combined time out of refrigeration should not exceed 3 days.

From a microbial point of view, unless the method of preparation of the syringe precludes the risk of microbial contamination, the product should be used immediately. If not used immediately, in-use storage times and conditions are the responsibility of the user.

### 6.4 Special Precautions for Storage

Store in a refrigerator (2° C.-8° C.).

Do not freeze.

Store in the original package in order to protect from light.

### 6.5 Nature and Contents of Container

5.6 mL solution in a 6 mL Type I glass vial with rubber stopper, aluminium seal and polypropylene flip-off cap.

Pack size of 1 vial.

### 6.6 Special Precautions for Disposal and Other Handling

Vyvgart comes as a ready-to-use solution in single-use vial. The medicinal product does not need to be diluted.

Visually inspect that the vial content is a yellowish, clear to opalescent solution, and devoid of particulate matter. If visible particles are observed the vial must not be used.

After removing the vial from the refrigerator, wait for at least 15 minutes before injecting to allow the solution to reach room temperature (see section 6.3).

Any unused medicinal product or waste material should be disposed of in accordance with local requirements.

### REFERENCES

1. Lehmann H C, Burke D, Kuwabara S. Chronic inflammatory demyelinating polyneuropathy: update on diagnosis, immunopathogenesis and treatment. J Neurol Neurosurg Psychiatry. 2019; 90(9):981-987.
2. Vallat J, Sommer C, Magy L. Chronic inflammatory demyelinating polyradiculoneuropathy: diagnostic and therapeutic challenges for a treatable condition. Lancet Neurol. 2010; 9(4):402-412.
3. Mathey E K, Park S B, Hughes R A C et al., Chronic inflammatory demyelinating polyradiculoneuropathy: from pathology to phenotype. J Neurol Neurosurg Psychiatry. 2015; 86(9):973-985.
4. Dalakas M. C. et al. Intravenous immunoglobulin in chronic inflammatory demyelinating polyradiculoneuropathy (CIDP): mechanisms of action and clinical and genetic considerations. Expert Review of Neurotherapeutics. 2022; 22(11-12): 953-962.
5. Bunschoten C et al. Progress in diagnosis and treatment of chronic inflammatory demyelinating polyradiculoneuropathy. Lancet Neurol. 2019; 18(8):784-794.
6. Kuwabara S, Misawa S, Mori M et al. Long term prognosis of chronic inflammatory demyelinating polyneuropathy: a five year follow up of 38 cases. J Neurol Neurosurg Psychiatry 2006; 77(1): 66-70.
7. Jefferis R and Lefranc M. Human immunoglobulin allotypes: possible implications for immunogenicity. mAbs. 2009; 1(4):332-338.
8. de Taeye S W, Bentlage A E H, Mebius M M et al. FcTR Binding and ADCC Activity of Human IgG Allotypes. Front Immunol. 2020; 11:740.
9. Edelman G M, Cunningham B A, Gall W E et al. The covalent structure of an entire gammaG immunoglobulin molecule. Proc Natl Acad Sci USA. 1969; 63(1):78-85.
10. Kabat et al. Sequences of Proteins of Immunological Interest U.S. Dept. Health and Human Services, 5th edition, 1991.
11. Vanhoutte E K, Faber C G, van Nes S I et al. Modifying the Medical Research Council grading system through Rasch analyses. Brain. 2012; 135:1639-1649.
12. Vaccaro C, Zhou J, Ober R J, et al. Engineering the Fc region of immunoglobulin G to modulate in vivo antibody levels. Nat Biotechnol. 2005; 23(10):1283
13. Shpilberg O and Jackisch C. Subcutaneous administration of rituximab (MabThera) and trastuzumab (Herceptin) using hyaluronidase. Br J Cancer. 2013; 109(6); 1556-1561.
14. Van den Bergh P Y K, Hadden R D M, Bouche P, et al. European Federation of Neurological Societies/Peripheral Nerve Society (EFNS/PNS) guideline on management of chronic inflammatory demyelinating polyradiculoneuropathy: Report of a joint task force of the European Federation of Neurological Societies and the Peripheral Nerve Society First Revision. Eur J Neurol. 2010; 17:356-363.
15. Van den Bergh P Y K, van Doorn P A, Hadden R D M, et al. European Academy of Neurology/Peripheral Nerve Society (EFNS/PNS) guideline on diagnosis and treatment of chronic inflammatory demyelinating polyradiculoneuropathy: Report of a joint Task Force-Second revision. Eur J Neurol. 2021; 28:3556-3583.
16. Merkies I S, Schmitz P I, van der Meché F G, et al. Clinimetric evaluation of a new overall disability scale in immune mediated polyneuropathies. J Neurol Neurosurg Psychiatry. 2002; 72:596-601.
17. van Nes S I, Vanhoutte E K, van Doom P A, et al. Rasch-built Overall Disability Scale (R-ODS) for immune-mediated peripheral neuropathies. Neurology. 2011; 76(4):337-345.
18. Vanhoutte E K, Latov N, Deng C, et al. Vigorimeter grip strength in CIDP: a responsive tool that rapidly measures the effect of IVIG—the ICE study. Eur J Neurol. 2013; 20(5):748-755.
19. Querol L, Nogales-Gadea G, Rojas-Garcia R, et al. Antibodies to contactin-1 in chronic inflammatory demyelinating polyneuropathy. Ann Neurol. 2013; 73(3):370-380.
20. Cortese A, Lombardi R, Briani C, et al. Antibodies to neurofascin, contactin-1 and contactin-associated protein 1 in CIDP: clinical relevance of IgG isotype. Neurol Neuroimmunol Neuroinflamm. 2020; 7:e639.
21. Pascual-Goñi E, Fehmi J, Lleixà C, et al. Antibodies to the Caspr1/contactin-1 complex in chronic inflammatory demyelinating polyneuropathy. Brain. 2021; 144(4):1183-1196.
22. Delmont E, Manso C, Querol L, et al. Autoantibodies to nodal isoforms of neurofascin in chronic inflammatory demyelinating polyneuropathy. Brain. 2017; 140(7):1851-1858.
23. van Schaik I N, Bril V, van Geloven N, et al. Subcutaneous immunoglobulin for maintenance treatment in chronic inflammatory demyelinating polyneuropathy (PATH): a randomised, double-blind, placebo-controlled, phase 3 trial. Lancet Neurol. 2018; 17(1):35-46.
24. Gorson K C, van Schaik I N, Merkies I S J, et al. Chronic inflammatory demyelinating polyneuropathy disease activity status: recommendations for clinical research standards and use in clinical practice. J Peripher Nerv Syst. 2010; 15:326-333.
25. Cleeland C S, Ryan K M. Pain assessment: Global use of the Brief Pain Inventory. Ann Acad Med Singapore. 1994; 23(2):129-38.
26. Atkinson M J, Sinha A, Hass S L, et al. Validation of a general measure of treatment satisfaction, the Treatment Satisfaction Questionnaire for Medication (TSQM), using a national panel study of chronic disease. Health Qual Life Outcomes. 2004; 2:12.
27. Atkinson M J, Kumar R, Cappelleri J C, et al. Hierarchical construct validity of the treatment satisfaction questionnaire for medication (TSQM version II) among outpatient pharmacy consumers. Value Health. 2005; 8 Suppl 1: S9-S24.
28. Bharmal M, Payne K, Atkinson M J, et al. Validation of an abbreviated Treatment Satisfaction Questionnaire for Medication (TSQM-9) among patients on antihypertensive medications. Health Qual Life Outcomes. 2009; 7:36.
29. Van Nes S I, Vanhoutte E K, Faber C G, et al. Improving fatigue assessment in immune-mediated neuropathies: the modified Rasch-built fatigue severity scale. J Peripher Nerv Syst. 2009; 14:268-278.
30. Zigmond A S, Snaith R P. The hospital anxiety and depression scale. Acta Psychiatr Scand. 1983; 67(6):361-370.
31. Bjelland I, Dahl A A, Haug T T, et al. The validity of the Hospital Anxiety and Depression Scale: An updated literature review. J Psychosom Res. 2002; 52(2):69-77.

All publications, patents, and patent applications mentioned in this specification are herein incorporated by reference to the same extent as if each individual publication, patent, or patent application was specifically and individually indicated to be incorporated by reference.

1. A method of treating Chronic Inflammatory Demyelinating Polyneuropathy (CIDP) in a subject in need thereof, the method comprising administering to the subject an effective amount of a human neonatal Fc receptor (FcRn) antagonist, wherein the FcRn antagonist comprises or consists of a variant Fc region or FcRn binding fragment thereof.

2-5. (canceled)

6. The method of claim 1, wherein the variant Fc region comprises or consists of a first Fc domain and a second Fc domain which form a homodimer or heterodimer, wherein the first Fc domain and/or the second Fc domain comprise amino acids Y, T, E, K, and F at EU positions 252, 254, 256, 433, and 434, respectively.

7-8. (canceled)

9. The method of claim 6, wherein the first Fc domain and/or the second Fc domain comprise an amino acid sequence independently selected from the group consisting of SEQ ID NO: 1, SEQ ID NO: 2, SEQ ID NO: 3, and SEQ ID NO: 4.

10. (canceled)

11. The method of claim 1, wherein the FcRn antagonist is efgartigimod.

12-16. (canceled)

17. The method of claim 1, wherein the FcRn antagonist is administered subcutaneously.

18. The method of claim 1, wherein the FcRn antagonist is administered to the subject once weekly, once every two weeks, once every three weeks, once every four weeks, once monthly, or once every six weeks.

19-20. (canceled)

21. The method of claim 1, wherein the FcRn antagonist is administered subcutaneously at a fixed dose of about 1000 mg once weekly.

22-25. (canceled)

26. The method of claim 1, wherein the FcRn antagonist is administered intravenously once weekly or once every two weeks.

27. The method of claim 26, wherein the FcRn antagonist is administered at a dose of from about 3 mg/kg to about 60 mg/kg.

28-36. (canceled)

37. The method of claim 1, wherein the FcRn antagonist is administered subcutaneously once weekly until the subject demonstrates evidence of clinical improvement (ECI) or demonstrates evidence of clinically meaningful deterioration (ECMD).

38. The method of claim 37, wherein the FcRn antagonist is administered subcutaneously once weekly until the subject demonstrates ECI during two consecutive measurements.

39. The method of claim 37, wherein ECI is a clinical improvement in one or more of Inflammatory Rasch-built Overall Disability Scale (I-RODS), Mean Grip Strength, or Inflammatory Neuropathy Cause and Treatment (INCAT) score.

40. (canceled)

41. The method of claim 37, wherein the ECMD is one or more of an increase in adjusted INCAT (aINCAT) score of ≥1 points, a decrease in I-RODS of ≥4 points (using the centile metric), or a decrease in mean grip strength of ≥8 kPa in one hand using a handheld vigorimeter.

42. (canceled)

43. The method of claim 41, wherein the ECMD is:
a) an increase in aINCAT score of ≥1 points during two consecutive measurements,
b) an increase in aINCAT score of ≥1 points twice in two weeks; and/or
c) an increase in aINCAT score of ≥2 points.

44-45. (canceled)

46. The method of claim 1, wherein the subject has:
a) been diagnosed with CIDP according to the EFNS/PNS 2010 diagnostic criteria;
b) typical CIDP;
c) a CIDP variant;
d) distal CIDP;
e) multifocal CIDP;
f) focal CIDP;
g) motor CIDP;
h) sensory CIDP;
i) progressive CIDP; and/or
j) relapsing CIDP.

47-55. (canceled)

56. The method of claim 1, wherein the subject:
a) has an aINCAT score, prior to administration of the FcRn antagonist, of 2 or more;
b) has an aINCAT score, prior to administration of the FcRn antagonist, of 5 or more;
c) has an I-RODS centile metric score, prior to administration of the FcRn antagonist, of 10 or more, optionally 20 or more, optionally 30 or more, optionally 40 or more, optionally 50 or more;
d) is newly-diagnosed with CIDP; and/or
e) is treatment-naïve.

57-60. (canceled)

61. The method of claim 1, wherein the subject has:
a) previously received treatment for CIDP;
b) previously received steroid treatment;
c) previously received IVIg or SCIg treatment; and/or
d) active disease despite treatment with corticosteroids or immunoglobulins.

62-64. (canceled)

65. The method of claim 1, wherein the subject is a CIDP patient characterized by:
a) the presence of anti-NF155 antibodies;
b) the presence of anti-CNTN1 antibodies;
c) the presence of anti-Caspr1 antibodies;
d) the presence of anti-NF140/186 antibodies;
e) the presence of anti-GM-1 antibodies; and/or
f) the presence of anti-LM-1 antibodies.

66-70. (canceled)

71. The method of claim 1, wherein control of disease activity, partial remission, or complete remission is achieved following administration of the FcRn antagonist.

72-75. (canceled)

76. The method of claim 71, wherein, once the control of disease activity, partial remission or complete remission is achieved, continued administration of the FcRn antagonist prevents deterioration of symptoms.

77-83. (canceled)

84. The method of claim 1, wherein the treatment reduces the risk of relapse by at least 60%.

85. The method of claim 1, wherein the subject shows an improvement in symptoms following administration of the FcRn antagonist, as measured using;
a) the INCAT score;
b) the aINCAT score;
c) the MRC sum score;
d) the I-RODS;
e) the Mean Grip Strength test; and/or
f) the TUG test.

86. The method of claim 85, wherein the subject shows:
a) a decrease in aINCAT score of 1 or more points;
b) an increase in I-RODS centile metric score of 4 or more points; and/or
c) an increase in mean grip strength of ≥8 kPa.

87-93. (canceled)

94. The method of claim 85, wherein an improvement in symptoms is achieved within 12 weeks or less, optionally within 8 weeks or less, optionally within 6 weeks or less, optionally within 4 weeks or less, optionally within 3 weeks or less, of first receiving the FcRn antagonist.

95-99. (canceled)

100. The method of claim 1, wherein the subject demonstrates ECI following administration of the FcRn antagonist.

101. The method of claim 100, wherein the subject demonstrates ECI within 31-51 days of first receiving the FcRn antagonist or within 43 days of first receiving the FcRn antagonist.

102. (canceled)

103. The method of claim 1, further comprising administering to the subject an effective amount of one or more additional therapeutic agents.

104-107. (canceled)

108. A method for treating CIDP in subjects in a patient population comprising:
administering 1008 mg/11,200 units of efgartigimod PH20, once weekly, wherein the patient population demonstrates ECI in 66.5% of the subjects in the patient population following administration of the efgartigimod PH20.

109. The method of claim 108, wherein the patient population:
a) achieves the ECI within 31-51 days of first receiving the efgartigimod PH20;
b) achieves the ECI within 43 days of first receiving the efgartigimod PH20; and/or
c) comprises 322 subjects.

110-111. (canceled)

112. The method of claim 108, wherein anti-efgartigimod alfa antibodies are detected in 6% of the patient population following administration of the efgartigimod PH20 for up to 12 weeks.

113. The method of claim 112, wherein neutralizing anti-efgartigimod alfa antibodies are detected in 0.3% of the patient population following administration of the efgartigimod PH20 for up to 12 weeks.

114. The method of claim 108, wherein the subjects in the patient population remain relapse-free significantly longer than subjects who did not receive efgartigimod PH20.

115. The method of claim 108, wherein the subjects in the patient population experience a longer time to clinical deterioration, which is statistically significant compared to subjects who did not receive efgartigimod PH20, wherein the clinical deterioration is:
a) an increase of ≥1 point in aINCAT score;
b) an increase of ≥1 point in aINCAT score during two consecutive measurements; and/or
c) an increase of 2 points in aINCAT score.

116-117. (canceled)

118. The method of claim 115, wherein the longer time to clinical deterioration is demonstrated by a hazard ratio of 0.394.

119. (canceled)

120. A method for treating CIDP in a subject comprising:
administering 1008 mg/11,200 units of efgartigimod PH20, once weekly, wherein the subject demonstrates ECI following administration of the efgartigimod PH20.

121. The method of claim 120, wherein the subject achieves the ECI within 31-51 days or within 43 days of first receiving the efgartigimod PH20.

122-123. (canceled)

124. The method of claim 120, wherein the subject demonstrates a reduced risk of showing ECMD.

125. The method of claim 124, wherein the ECMD is:
a) an increase in aINCAT score of ≥1 points;
b) an increase in aINCAT score of ≥1 points during two consecutive measurements;
c) an increase in aINCAT score of ≥1 points twice in two weeks; and/or
d) an increase in aINCAT score of ≥2 points.

126-128. (canceled)

129. A method for treating CIDP in subjects in a patient population comprising:
administering 1008 mg/11,200 units of efgartigimod PH20, once weekly, wherein mean percentage reduction from baseline in total IgG levels ranged between 66.8% and 71.6% in the patient population following administration of the efgartigimod PH20.

130-132. (canceled)

133. A method for treating CIDP in a subject comprising:
administering 1008 mg/11,200 units of efgartigimod PH20, once weekly, wherein the subject shows a reduction in serum level of total IgG of between 66.8% and 71.6% following administration of the efgartigimod PH20, compared to a baseline value prior to administration of the efgartigimod PH20.

134-135. (canceled)

136. A method for treating CIDP in subjects in a patient population comprising:
administering 1008 mg/11,200 units of efgartigimod PH20, once weekly, wherein the subjects in the patient population experience a longer time to clinical deterioration, which is statistically significant compared to subjects who did not receive efgartigimod PH20, wherein the clinical deterioration is an increase of ≥1 point in aINCAT score.

137. The method of claim 136, wherein the longer time to clinical deterioration is demonstrated by a hazard ratio of 0.394.

138. The method of claim 136, wherein the patient population comprises 221 subjects.

139. A method of treating CIDP in a subject in need thereof, the method comprising subcutaneously administering to the subject a clinically proven safe and a clinically proven effective amount of efgartigimod PH20 once weekly.

140. The method of claim 139, wherein the clinically proven safe and clinically proven effective amount of efgartigimod PH20 is administered subcutaneously over approximately 30 to 90 seconds.

141. The method of claim 139, wherein the clinically proven safe and clinically proven effective amount of efgartigimod PH20 is 1008 mg/11,200 units.

142. The method of claim 139, wherein the subject demonstrates evidence of improvement following administration of the efgartigimod PH20.

143. The method of claim 142, wherein the subject demonstrates evidence of improvement during two consecutive measurements.

144. The method of claim 142, wherein the evidence of improvement is selected from aINCAT improvement ≥1 point, I-RODS improvement ≥4 points, or mean grip strength improvement ≥8 kPa.

145. The method of claim 139, wherein the subject experiences a longer time to clinical deterioration following administration of the efgartigimod PH20 as compared to a subject that does not receive efgartigimod PH20.

146. The method of claim 145, wherein the clinical deterioration is:
a) an increase in aINCAT score of ≥1 points;
b) an increase in aINCAT score of ≥1 points during two consecutive measurements;
c) an increase in aINCAT score of ≥1 points twice in two weeks; and/or
d) an increase in aINCAT score of 2 points.

147-149. (canceled)

150. A method of treating CIDP in subjects in a patient population, the method comprising subcutaneously administering a clinically proven safe and a clinically proven effective amount of efgartigimod PH20 once weekly.

151. The method of claim 150, wherein the clinically proven safe and clinically proven effective amount of efgartigimod PH20 is administered subcutaneously over approximately 30 to 90 seconds.

152. The method of claim 150, wherein the clinically proven safe and clinically proven effective amount of efgartigimod PH20 is 1008 mg/11,200 units.

153. The method of claim 150, wherein the patient population demonstrates evidence of improvement during two consecutive measurements in 69% of the subjects in the patient population following administration of the efgartigimod PH20.

154. The method of claim 153, wherein the evidence of improvement is selected from aINCAT improvement ≥1 point, I-RODS improvement ≥4 points, or mean grip strength improvement ≥8 kPa.

155. The method of claim 150, wherein the subjects in the patient population experience a longer time to clinical deterioration compared to subjects who did not receive efgartigimod PH20, wherein the clinical deterioration is:
a) an increase of ≥1 point in aINCAT score;
b) an increase in aINCAT score of ≥1 points during two consecutive measurements; and/or


SEQUENCE LISTING (33 sequences)

SEQ ID NO: 1 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 222 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 5 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 6 - protein, 226 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 7 - protein, 225 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 8 - protein, 224 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 9 - protein, 225 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 10 - protein, 224 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 11 - protein, 223 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 12 - protein, 223 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 13 - protein, 222 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 14 - protein, 221 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 15 - protein, 221 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 16 - protein, 220 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 17 - protein, 219 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 18 - protein, 224 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 19 - protein, 223 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 20 - protein, 222 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 21 - protein, 220 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 22 - protein, 227 aa, synthetic construct
  VARIANT 1: note = D or absent
  VARIANT 2: note = K or absent
  VARIANT 3: note = T or absent
  VARIANT 4: note = H or absent
  VARIANT 5: note = T or absent
  VARIANT 6: note = C or absent
  VARIANT 226: note = G or absent
  VARIANT 227: note = K or absent
XXXXXXPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPXX

SEQ ID NO: 23 - protein, 216 aa, synthetic construct
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 24 - protein, 445 aa, synthetic construct
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 219 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 214 aa, synthetic construct
DIQMTQSPSSLSASVGDRVTITCKASDHINNWLAWYQQKPGQAPRLLISGATSLETGVPSRFSGSGTGKDYTLTISSLQPEDFATYYCQQYWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 444 aa, synthetic construct
QVQLVQSGAELKKPGASVKLSCKASGYTFTSYGISWVKQATGQGLEWIGEIYPRSGNTYYNEKFKGRATLTADKSTSTAYMELRSLRSEDSAVYFCARSTTVRPPGIWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 15 aa, synthetic construct
EPKSCDKTHTCPPCP

SEQ ID NO: 32 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 33 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCPNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY